(19) United States
(12) Patent Application Publication
(10) Pub. No.: US 2019/0055563 A1
(43) Pub. Date: Feb. 21, 2019

(54) POLYMERASE Q AS A TARGET IN HR-DEFICIENT CANCERS

(71) Applicant: DANA-FARBER CANCER INSTITUTE, INC., Boston, MA (US)

(72) Inventors: Alan D. D'Andrea, Winchester, MA (US); Raphael Ceccaldi, Brookline, MA (US)

(21) Appl. No.: 15/768,853

(22) PCT Filed: Oct. 19, 2016
(86) PCT No.: PCT/US2016/057686

§ 371 Date: Apr. 17, 2018

Related U.S. Application Data
(60) Provisional application No. 62/243,330, filed on Oct. 19, 2015.

(51) Int. Cl. C12N 15/113; A61P 35/00; A61K 45/06; C12Q 1/6876; A61K 31/713; A61K 33/24; A61K 39/395; C07K 16/40

(52) U.S. Cl. CPC C12N 15/1137; A61P 35/00; A61K 45/06; C12Q 1/6876; A61K 31/713; C12N 2310/531; A61K 39/3955; C07K 16/40; C12Q 2600/136; C12N 2310/14; C12N 2310/122; A61K 33/24

(57) ABSTRACT

The disclosure relates, in some aspects, to methods of treating homologous recombination (HR)-deficient cancers. In some embodiments, the disclosure provides method for treating HR-deficient cancer by administering a polymerase Q (PolQ) inhibitor.

### RELATED APPLICATIONS

This applications claims priority under 35 U.S.C. § 119(e) to U.S. provisional application No. 62/243,330, filed Oct. 19, 2015, the contents of which are incorporated herein by reference in its entirety.

### GOVERNMENT SUPPORT

This invention was made with government support under grant numbers RO1 DK043889 and R37 HL052725 awarded by The National Institutes of Health. The government has certain rights in the invention.

### BACKGROUND OF THE INVENTION

Large-scale genomic studies have shown that half of epithelial ovarian cancers (EOCs) have alterations in genes regulating homologous recombination (HR) repair. Loss of HR accounts for the genomic instability of EOCs and for their cellular hyper-dependence on alternative poly-ADP ribose polymerase (PARP)-mediated DNA repair mechanisms. PARP inhibitors (PARPi) can be used to treat some HR-deficient cancers. However, certain cancers are resistant to treatment with PARP inhibitors. Accordingly, there is a general need to develop novel methods of regulating DNA repair mechanisms for the treatment of HR-deficient cancer.

### SUMMARY OF THE INVENTION

Aspects of the disclosure relate, in part, to the surprising discovery that an inverse relationship exists between homologous recombination (HR) and DNA polymerase θ (Polθ)-mediated repair mechanisms. In certain aspects, the invention relates to the discovery that blockade of Polθ activity leads to enhanced death of HR-deficient cancer cells.

Accordingly, in some aspects, the disclosure provides a method for treating homologous recombination (HR)-deficient cancer in a subject, the method comprising: administering to the subject in need thereof a DNA polymerase θ (Polθ) inhibitor in an amount effective to treat the HR-deficient cancer. In some embodiments, the HR-deficient cancer is resistant to treatment with a poly (ADP-ribose) polymerase (PARP) inhibitor alone.

Certain aspects of the disclosure relate, in part, to the surprising discovery that Polθ inhibitor(s) are also useful in treating cancers that are resistant to PARP inhibitor therapy. Therefore, in some aspects, the disclosure provides a method for treating a cancer that is resistant to poly (ADP-ribose) polymerase (PARP) inhibitor therapy in a subject, the method comprising: administering to the subject in need thereof a DNA polymerase θ (Polθ) inhibitor in an amount effective to treat the PARP inhibitor-resistant cancer. In some embodiments, the PARP inhibitor-resistant cancer is deficient in homologous recombination.

The inventors have also recognized and appreciated that Polθ expression is up-regulated in certain cancers (e.g., ovarian cancer, cervical cancer, breast cancer). Thus, in some aspects, the disclosure provides a method for treating a cancer that is characterized by overexpression of DNA polymerase θ (Polθ) in a subject, the method comprising: administering to the subject in need thereof a DNA polymerase θ (Polθ) inhibitor in an amount effective to treat the Polθ-overexpressing cancer. In some embodiments, the Polθ-overexpressing cancer is deficient in homologous recombination.

Mutation of certain genes (e.g., BRCA genes, genes encoding Fanconi proteins) are correlated with HR-deficiency in some cancers. In some aspects, the disclosure provides a method for treating a cancer that is characterized by one or more BRCA mutations and/or reduced expression of Fanconi (Fanc) proteins in a subject, the method comprising: administering to the subject in need thereof a DNA polymerase θ (Polθ) inhibitor in an amount effective to treat the cancer. In some embodiments, the cancer characterized by one or more BRCA mutations and/or reduced expression of Fanconi (Fanc) proteins is also characterized by overexpression of DNA polymerase θ (Polθ).

In some embodiments, a method described by the disclosure further comprises treating the subject with one or more anti-cancer therapy.

In some embodiments, the anti-cancer therapy is selected from the group consisting of surgery, radiation therapy, chemotherapy, gene therapy, DNA therapy, viral therapy, RNA therapy, adjuvant therapy, and immunotherapy. In some embodiments, the chemotherapy comprises administering to the subject a cytotoxic agent in an amount effective to treat the HR-deficient cancer.

In some embodiments, the Polθ inhibitor and the anti-cancer therapy are synergistic in treating the cancer, compared to the Polθ inhibitor alone or the anti-cancer therapy alone.

In some embodiments, the Polθ inhibitor is a small molecule, antibody, peptide or antisense compound.

In some embodiments, the cytotoxic agent is selected from the group consisting of a platinum agent, mitomycin C, a poly (ADP-ribose) polymerase (PARP) inhibitor, a radioisotope, a vinca alkaloid, an antitumor alkylating agent, a monoclonal antibody and an antimetabolite.

In some embodiments, the Polθ inhibitor and the anti-cancer therapy are administered concurrently or sequentially.

Methods of identifying Polθ inhibitors are also contemplated by the disclosure. In some aspects, the disclosure provides a high-throughput screening method for identifying an inhibitor of ATPase activity of DNA polymerase θ (Polθ), the method comprising: contacting Polθ or a fragment thereof with adenosine triphosphate (ATP) and single-stranded DNA (ssDNA) substrate in the presence and absence of a candidate compound; quantifying amount of adenosine diphosphate (ADP) produced in the presence and absence of the candidate compound; and, identifying the candidate compound as an inhibitor of the ATPase activity of Polθ if the amount of ADP produced in the presence of the candidate compound is less than the amount produced in the absence of candidate compound.

In some embodiments, the amount of ADP produced is quantified using luminescence or radioactivity. In some embodiments, the amount of ADP is quantified using the ADP-Glo™ Kinase assay.

In some embodiments, the Polθ or fragment thereof, ATP and ssDNA substrate are incubated in the presence or absence of the candidate compound for at least 2 hours, 4 hours, 8 hours, 10 hours, 12 hours, 14 hours, 16 hours, or 18 hours. In some embodiments, the Polθ fragment comprises N-terminal ATPase domain of Po10.

In some embodiments, 5 nM, 10 nM, or 15 nM of Polθ or a fragment thereof is used. In some embodiments, 25, 50, 100, 125, 150, or 175 μM of ATP is used.

In some embodiments, the candidate compound is a small molecule, antibody, peptide or antisense compound.

Each of the embodiments and aspects of the invention can be practiced independently or combined. Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

These and other aspects of the inventions, as well as various advantages and utilities will be apparent with reference to the Detailed Description. Each aspect of the invention can encompass various embodiments as will be understood.

All documents identified in this application are incorporated in their entirety herein by reference.

### BRIEF DESCRIPTION OF THE DRAWINGS

FIGS. 1A-1G. POLQ is a RAD51-interacting protein that suppresses HR. FIG. 1A, DR-GFP assay in U2OS cells transfected with indicated siRNA. FIG. 1B, Quantification of RAD51 foci in U2OS cells transfected with indicated siRNA. FIG. 1C, Endogenous RAD51 co-precipitates in vivo with purified full-length Flag-tagged POLQ from whole cell extracts. FIG. 1D, GST pull-down experiment with full-length Flag-tagged POLQ. (*: non-specific band). FIG. 1E, GST-RAD51 pull-down with in-vitro translated POLQ truncation mutants. FIG. 1F, GST-RAD51 pull-down with in-vitro translated POLQ versions missing indicated amino acids. FIG. 1G, Ponceau staining and immunoblotting of peptide arrays for the indicated POLQ motifs probed with recombinant RAD51. The POLQ amino acids spanning RAD51-interacting motifs are shown. 1-POLQ sequences are SEQ ID NOs: 19-31 from top to bottom; 2-POLQ sequences are SEQ ID NOs: 32-45; and 3-POLQ sequences are SEQ ID NOs: 46-61. Data in FIGS. 1A and 1B represent mean±s.e.m.

FIGS. 2A-2H. POLQ inhibits RAD51-mediated recombination. FIG. 2A, Schematic of POLQ mutants used in complementation studies and their interaction with RAD51. FIG. 2B, Quantification of RAD51 foci in U2OS cells transfected with indicated siRNA and POLQ cDNA constructs refractory to siPOLQ1. FIG. 2C, DR-GFP assay in U2OS cells transfected with indicated siRNA and POLQ cDNA constructs refractory to siPOLQ1. FIG. 2D, Coomassie-stained gel of the purified POLQ fragment. FIG. 2E, Quantification of POLQ ATPase activity. FIG. 2F, Quantification of POLQ binding to ssDNA and dsDNA. FIG. 2G, RAD51-ssDNA nucleofilament assembly assay. FIG. 2H, Assessment of RAD51-dependent D-loop formation. Data in FIGS. 2B, 2C, 2E, and 2F represent mean±s.e.m.

FIGS. 3A-3G. POLQ promotes S phase progression and recovery of stalled forks. FIG. 3A, POLQ gene expression in subtypes of cancers with HR deficiency. FIG. 3B, Survival assays of A2780 cells exposed to the indicated DNA-damaging agents. Immunoblot showing silencing efficiency. FIG. 3C, Immunoblot analyses following pulse treatments with DNA-damaging agents (*γH2AX: see methods). FIG. 3D, Cell cycle progression of synchronized A2780 cells. A representative cell cycle distribution. FIG. 3E, Fraction of cycling A2780 cells measured by EdU incorporation. FIG. 3F, Quantification of DNA fiber lengths. FIG. 3G, Percentage of stalled forks. All experiments shown in FIGS. 3A-3D were performed in two cell lines (A2780 and 293T). All data represent mean±s.e.m. except for box plots in f that show twenty-fifth to seventy-fifth percentiles, with lines indicating the median, and whiskers indicating the smallest and largest values.

FIGS. 4A-4J. Synthetic lethality between HR and POLQ repair pathways. FIG. 4A, Clonogenic formation of BRCA1-deficient (MDA-MB-436) cells expressing indicated cDNA together with indicated shRNA. FIG. 4B, Chromosome breakage analysis of HR-deficient cells transfected with the indicated siRNA. A representative image is shown. Arrows indicate chromosomal aberrations. FIG. 4C, Embryos at day 14 of gestation. FIG. 4D, Growth of indicated xenografts in vivo. Immunoblot showing silencing efficiency. FIG. 4E, Relative tumor volumes (RTV) for individual mice treated in (FIG. 4D) after three weeks of treatment. FIG. 4F, Overall survival for mice treated with vehicle or PARPi. Log-rank P<10⁻³. Clonogenic formation (FIG. 4G) and chromosome breakage analysis (FIG. 4H) of BRCA2-deficient cells expressing POLQ cDNA constructs refractory to siPOLQ1 and transfected with the indicated siRNA. FIG. 4I, Clonogenic formation of BRCA2-deficient cells transfected with the indicated siRNA. FIG. 4J, Model for role of POLQ in DNA repair. Data in FIGS. 4A, 4B, 4G, and 4I represent mean±s.e.m. For data in FIGS. 4D-4F, each circle represents data from one tumor and each group represents n≥7 tumors from n≥6 mice. Brackets show mean±s.e.m.

FIGS. 5A-5L. POLQ is highly expressed in epithelial ovarian cancers (EOCs) and POLQ expression correlates with expression of HR genes. Gene set enrichment analysis (GSEA) for expression of TransLesion Synthesis (TLS) (FIG. 5A) and polymerase (FIG. 5B) genes between primary cancers and control samples in 28 independent datasets from 19 different cancers types. Enrichment values (represented as a single dot for each gene in a defined dataset) were determined using the rank metric score to compare expression values between cancers and control samples. Dots above the dashed line reflect enrichment in cancer samples, whereas dots below the dashed line show gene expression enriched in control samples. Datasets were ranked based on the amplitude of the rank metric score and plotted as shown. FIG. 5C, POLQ gene expression in 40 independent datasets from 19 different cancer types. For each dataset, POLQ values were expressed as fold-change differences relative to the mean expression in control samples, which was arbitrarily set to 1. FIG. 5D, POLQ expression correlates with tumor grade and MKi67 gene expression in the ovarian TCGA (n=494 patients with ovarian carcinoma (grade 1, n=5; grade 2, n=61; grade 3, n=428) and control samples, n=8). FIG. 5E, POLQ expression correlates with tumor grade MKi67 gene expression in the ovarian dataset GSE9891 (n=251 patients with ovarian serous and endometrious carcinoma for which grade status was available (grade 1, n=20; grade 2, n=88; grade 3, n=143)). Statistical correlation was assessed using the Pearson test (for d: r=0.65, P<10⁻³; for e: r=0.77, P<10⁻³). FIG. 5F, Top-ranked biological pathways differentially expressed between samples expressing high levels of POLQ (high POLQ, 1^{st} 33%, n=95) relative to samples with low POLQ expression (low POLQ, 67%, n=190) on the ovarian dataset GSE9891 (n=285 patients with ovarian carcinoma). Significance values were determined by the hypergeometrical test using the 200 most differentially expressed probe sets between the 2 groups (high POLQ and low POLQ). FIG. 5G, GSEA for expression of DNA repair genes between primary cancers and control samples in 5 independent ovarian cancer datasets. A representative heat map showing differential gene expression between ovarian cancers and controls is shown from GSE14407. For each dataset, DNA repair genes were ranked based on the metric score reflecting their enrichment in cancer samples. The top 20 DNA repair genes primarily expressed in cancer samples compared to control samples is shown on the right. FIG. 5H, GSEA for the top 20 DNA repair genes defined in (FIG. 5G) between primary cancers and control samples in 40 independent cancer datasets. The nominal P-value was used as a measure of the expression enrichment in cancer samples and represented as a waterfall plot. When the gene set expression was enriched in control samples, the P-value was arbitrarily set to 1.

FIG. 5I, POLQ expression correlates with RAD51 and FANCD2 gene expression in 285 samples from the ovarian dataset GSE9891. Statistical correlation was assessed using the Pearson test (r=0.71, P<10⁻³). FIG. 5J, Top 10 genes that most closely correlated with POLQ expression (gene neighbors analysis) for 1046 cell lines from the CCLE collection. DNA repair activity for these genes is indicated in the table. Increased HR gene expression is known to positively correlate with improved response to platinum based chemotherapy (a surrogate of HR deficiency) and thus can be predictive of decreased HR activity^{31,38}. Conceptually, a state of HR deficiency may lead to compensatory increased expression of other HR genes. FIG. 5K, Top-ranked Gene Ontology (GO) terms for the molecular functions encoded by the top 20 DNA repair genes defined in FIGS. 5G and 5L. Schematic representation of POLQ domain structure with the helicases (BLM, SEQ ID NOs: 64-65 from left to right, RECQL4, SEQ ID NOs: 66-67 from left to right, RAD54B, SEQ ID NOs: 68-69 from left to right, and RAD54L, SEQ ID NOs: 70-71 from left to right) that co-expressed with POLQ (SEQ ID NOs: 62-63 from left to right) (FIG. 5L). Conserved amino-acid sequences of ATP binding and hydrolysis motifs (namely Walker A and B) are indicated. Cox plots in FIG. 5C that show twenty-fifth to seventy-fifth percentiles, with lines indicating the median, and whiskers indicating the smallest and largest values. For FIGS. 5D and 5E (top panels), each dot represents the expression value from one patient, brackets show mean±s.e.m.

FIGS. 6A-6I. POLQ is a RAD51-interacting protein required for maintenance of genomic stability. FIG. 6A, siRNA sequences (siPOLQ1 and siPOLQ2) efficiently down-regulate exogenously transfected POLQ protein. POLQ levels were detected by immunoblotting with Flag or POLQ antibody (left) and by RT-qPCR using 2 different sets of POLQ primers (right). The asterisk on the immunoblot indicates a non-specific band. Expression was normalized using GAPDH as a reference gene. POLQ gene expression values are displayed as fold-change differences relative to the mean expression in control cells, which was arbitrarily set to 1. FIG. 6B, Quantification of baseline and HU-induced γH2AX foci in U2OS cells transfected with indicated siRNA.

FIG. 6C, Quantification of IR-induced RAD51 foci in BrdU-positive U2OS cells transfected with indicated siRNA. FIG. 6D, POLQ inhibition by siRNA induced a decrease in the cellular survival of 293T cells treated with MMC in a 3-day survival assay. FIG. 6E, Quantification of chromosomal aberrations in 293T cells transfected with indicated siRNA. FIG. 6F, Schematic representation of POLQ truncation proteins used for RAD51 interaction studies. FIG. 6G, Endogenous RAD51 co-precipitates with Flag-tagged POLQ-ΔPol1 (POLQ-1-1416) but not POLQ-1633-Cter, each stably expressed in HeLa cells. FIG. 6H, Sequence alignment between the RAD51-interacting motifs of C. elegans RFS-1 (SEQ ID NO: 72) and human POLQ (SEQ ID NO: 73). FIG. 6I, Schematic of POLQ domain structure with its homologs HELQ and POLN. All data show mean±s.e.m.

FIGS. 7A-7D. Characterization of RAD51-interacting motifs in POLQ. FIG. 7A, GST-RAD51 pull-down with in vitro-translated POLQ proteins missing indicated amino acids. FIG. 7B, Schematic of POLQ mutants used in complementation studies. FIG. 7C, Quantification of IR-induced RAD51 foci in U2OS cells stably integrated with empty vector (EV) or POLQ-ΔPol1 cDNA, that is refractory to siPOLQ1. Cells were transfected with indicated siRNA and subsequently treated with IR. The number of cells with more than 10 RAD51 foci was calculated relative to control cells (si Scr). FIG. 7D, DR-GFP assay in U2OS cells stably integrated with empty vector (EV) or indicated POLQ cDNA constructs refractory to siPOLQ1 and transfected with indicated siRNA. All data show mean±s.e.m.

FIGS. 8A-8I. POLQ is an ATPase that suppresses RAD51-ssDNA nucleofilament assembly and formation of RAD51-dependent D-loop structures. FIG. 8A, Representative ΔPol2 WT radiometric ATPase assay. FIG. 8B, Gel mobility shift assays with ΔPol2 WT and ssDNA. FIG. 8C, Coomassie-stained gel showing the purified ΔPol2-A-dead fragment. FIG. 8D, Representative ΔPol2-A-dead radiometric ATPase assay. FIG. 8E, Quantification of ΔPol2-A-dead ATPase activity. (ssDNA: single-stranded DNA; dsDNA: double-stranded DNA). FIG. 8F, Assembly/disruption of RAD51-ssDNA filaments in the presence of increasing amounts of ΔPol2 WT. The order in which each component was added to the reaction is noted above. FIG. 8G, Schematics of the formation of RAD51-dependent D-loop structures. FIG. 8H, Formation of RAD51-containing D-loop structures following the addition of increasing amounts of ΔPol2 WT. FIG. 8I, Fraction of D-loop formed following the addition of increasing amounts of ΔPol2 WT. Data in FIG. 8I shows mean±s.e.m.

FIGS. 9A-9I. POLQ functions under replicative stress and is induced by HR deficiency. FIG. 9A, POLQ recruitment to the chromatin is enhanced by UV treatment. HeLa cells stably integrated with either Flag-tagged ΔPol1 or POLQ-1633-Cter (FIG. 6F) were subjected to UV treatment. Cells were collected at indicated time points after UV treatment and IPs were performed on nuclear and chromatin fractions. FIG. 9B, HeLa cells stably integrated with ΔPol1 were treated with UV and harvested at indicated time points following UV exposure. POLQ and RAD51 co-precipitation is enhanced by UV treatment. FIG. 9C, Quantification of DNA fiber lengths isolated from WT or Polq^{-/-} MEFs. FIG. 9D, Quantification of DNA fiber lengths isolated from WT or Polq^{-/-} MEFs transfected with either EV, or POLQ cDNA constructs. FIG. 9E, POLQ gene expression was analyzed by RT-qPCR in HR-deficient ovarian cancer cell lines (PEO-1 and UWB1-289) compared with other ovarian cancer cell lines, HeLa (cervical cancer) cells and 293T (transformed human embryonic kidney) cells. Expression was normalized using GAPDH gene as a reference. POLQ expression values are displayed as fold-change relative to the mean expression in HR-proficient control cells, which was arbitrarily set to 1. FIG. 9F, POLQ gene expression analysis (RT-qPCR) in 293T cells transfected with siRNA targeting FANCD2, BRCA1 or BRCA2 (left panel) and in corrected PD20 cells (PD20+FANCD2) relative to FANCD2-deficient cells (PD20) (right panel). Expression was normalized using GAPDH gene as a reference. POLQ expression values are presented as fold-change relative to the mean expression in control cells, which was arbitrarily set to 1. FIG. 9G, POLQ gene expression in 5 datasets of serous epithelial ovarian carcinoma (frequently associated with an HR deficiency) and 1 dataset of clear cell ovarian carcinoma (subgroup not associated with HR alterations). For each dataset, POLQ expression values are displayed as fold-change differences relative to the mean expression in control samples, which was arbitrarily set to 1. FIG. 9H, Progression-free survival (PFS) after first line platinum chemotherapy for patients with ovarian carcinoma (ovarian carcinoma TCGA). Statistical significance was assessed by the Log-Rank test (P<10⁻²). FIG. 9I, Effect of siPOLQ and the different POLQ cDNA constructs on HR read-out. NA: not applicable. Box plots in FIGS. 9C, 9D, and 9G show twenty-fifth to seventy-fifth percentiles, with lines indicating the median, and whiskers indicating the smallest and largest values. Data in FIGS. 9E and 9F show mean±s.e.m.

FIGS. 10A-10I. POLQ inhibition sensitizes HR-deficient tumors to cytotoxic drug exposure. Clonogenic formation of A2780 cells expressing Scrambled (Scr) shRNA or shRNAs against FANCD2 or BRCA2 with increasing amounts of MMC (FIG. 10A), UV (FIG. 10B) or IR (FIG. 10C). Clonogenic formation of A2780 cells expressing Scrambled (Scr) or FANCD2 shRNA, together with shRNA targeting POLQ, in increasing concentrations of CDDP (FIG. 10D), MMC (FIG. 10E) or PARPi (FIG. 10F). FIG. 10G, Inhibition of POLQ reduces the survival of A2780 cells after 3 days of continuous exposure to the ATM inhibitor Ku55933. FIG. 10H, Immunoblot analyses in A2780 cells expressing FANCD2 shRNA together with siRNA targeting POLQ or Scr at 24 hours after indicated MMC pulse treatment. FIG. 10I, FANCA-deficient fibroblasts (GM6418) were infected with a whole-genome shRNA library and treated with MMC for 7 days. The fold-change enrichment of each shRNA after MMC treatment was determined by sequencing relative to the infected cells before treatment. TP53 depletion is known to improve survival of FANCA^{-/-} cells³³. WRN depletion has recently been shown to be synthetically lethal with HR deficiency³⁹. Each column represents the mean of at least 2 independent shRNAs. All data show mean±s.e.m.

FIGS. 11A-11H. HR and POLQ repair pathways are synthetically lethal in vivo. FIG. 11A, Clonogenic formation of WT, Fancd2^{-/-}, Polq^{-/-} and Fancd2^{-/-}Polq^{-/-} MEFs with increasing concentrations of PARPi. FIG. 11B, A2780 cells were transduced with indicated shRNAs and xenotransplanted into both flanks of athymic nude mice. The tumor volumes for individual mice were measured biweekly for 8 weeks. Each group represents n≥5 tumors from n≥5 mice. FIG. 11C, Ki67 and γH2AX quantification in tumors treated with either vehicle or PARPi. FIG. 11D, Representative Ki67 and γH2AX staining of A2780-shFANCD2 xenografts expressing sh Scr or sh POLQ in athymic nude mice, treated with either vehicle or PARPi. Scale bars, 100 μM. FIG. 11E, In vivo competition assay design. FIG. 11F, Tumor chimerism post xenotransplantation for indicated conditions. FIG. 11G, Representative flow cytometry analysis of tumors before xenotransplantation (post FACS sorting) or after xenotransplantation (post-transplant, PARPi). The percentage of GFP-RFP cells is indicated. FIG. 11H, Tumor chimerism post xenotransplantation for indicated conditions. For Data in FIGS. 11F and 11H, each circle represents data from one tumor and each group represents n≥7 tumors from n≥6 mice. Brackets show mean±s.e.m. Data in FIGS. 11A-11C show mean±s.e.m. For f each group represents n≥6 tumors from n≥6 mice.

FIGS. 12A-12F. POLQ is required for HR-deficient cell survival and limits the formation of RAD51 structures in HR-deficient cells. FIG. 12A, Clonogenic formation of Fancd2^{--/-} Polq^{-/-} MEFs transfected with full-length POLQ cDNA constructs in the presence of increasing concentrations of PARPi. FIG. 12B, Chromosome breakage analysis of FANCD2-depleted cells that were first transfected with the indicated siRNA and full-length POLQ cDNA constructs refractory to siPOLQ1 and then exposed to MMC. FIG. 12C, DR-GFP assay in U2OS cells transfected with indicated siRNA. FIG. 2D, Quantification of baseline and IR-induced RAD51 foci in U2OS cells transfected with indicated siRNA. FIG. 12E, RAD51 recruitment to chromatin is enhanced by UV treatment. Vu423 cells (BRCA2^{-/-}) were collected at indicated time points after UV treatment and immunoblotting performed on the cytoplasmic, nuclear and chromatin fractions. FIG. 12F, RAD51 recruitment to chromatin in Vu423 cells (BRCA2^{-/-}) transfected with indicated siRNA. Histone H3 was used as a control for chromatin fractionation. All data show mean±s.e.m.

FIGS. 13A-13E. POLQ participates in error-prone DNA repair. FIG. 13A, End-joining reporter assay in U2OS cells transfected with indicated siRNA and/or treated with PARPi. FIG. 13B, End-joining reporter assay in U2OS cells transfected with indicated siRNA and POLQ cDNA constructs refractory to siPOLQ1. FIG. 13C, UV damage-induced POLQ foci formation in U2OS cells. POLQ foci were abolished by pre-treatment with PARPi. FIG. 13D, Mutation frequency was determined in damaged supF plasmid, recovered from siRNA-treated 293T cells. FIG. 13E, Non-synonymous mutation count in ovarian, uterine and breast TCGA. All data show mean±s.e.m.

FIGS. 14A-14B. Model depicting the role of POLQ in DNA repair. FIG. 14A, Mechanistic model for how POLQ limits RAD51-ssDNA filament assembly. According to this model, the ATPase domain of POLQ may prevent the assembly of RAD51 monomers into RAD51 polymers, perhaps by depleting local ATP concentrations. The RAD51 binding domains in the central region of POLQ may then sequester the RAD51 monomers, preventing filament assembly. FIG. 14B, I. Under physiological conditions, POLQ expression is low and its impact on repair of DNA double-strand breaks (DSB) is limited. II. When HR deficiency occurs, POLQ is then highly expressed and channels DSB repair toward alt-EJ. III. In the case of an HR-defect, the loss of POLQ leads to cell death through the persistence of toxic RAD51 intermediates and inhibition of alt-EJ.

FIGS. 15A-15B. Screening for inhibitors of the ATPase activity of Polθ. FIG. 15A, flowchart depicting one embodiment of a screening protocol for inhibitors of the ATPase activity of Polθ. FIG. 15B, Characterization of the ATP hydrolysis activity of purified Polθ fragment using the ADP-Glo kinase assay (Promega). Columns 1 and 2 show the normalized ADP-Glo luminescence signals from reactions lacking either ATP or the affinity-purified Polθ-ΔPol2 enzyme, respectively. Polθ-ΔPol2 (10 nM) was incubated for 16 hours in a reaction mixture containing ATP (100 μM) and either no ssDNA or 600 nM ssDNA (columns 3 and 4 respectively). Luminescence signals were normalized relative to the reaction lacking Polθ-ΔPol2 (column 2).

FIGS. 16A-16C. Adapting Polθ (ΔPol2) protein purification to a method using SF9 cells cultured in spinner flasks. FIG. 16A, Side-by-side comparison of Polθ (ΔPol2) protein yield obtain from SF9 cultured in 15 cm plates and from spinner flasks. FIG. 16B, Coomassie-stained gel of the purified Polθ (ΔPol2) fragment obtained from spinner flasks. FIG. 16C, Side-by-side quantification of ATPase activity of Polθ (ΔPol2) fragments purified by culture plates and spinner flasks. The ATPase activity was measured using the ADP Glo kit.

### DETAILED DESCRIPTION OF THE INVENTION

The present disclosure provides methods for treating homologous recombination (HR)-deficient and poly (ADP-ribose) polymerase (PARP)-resistant cancers. High-throughput screening methods for identifying inhibitors of interest are also provided.

It has been found, in accordance with the invention, that an inverse relationship exists between homologous recombination (HR) activity and DNA polymerase θ (Polθ) expression. Knockdown of Polθ was, surprisingly, found to enhance cell death in HR-deficient cancers. Consistent with these results, genetic inactivation of an HR gene (Fancd2) and Polθ in mice was found to result in embryonic lethality.

Accordingly, aspects of the disclosure relate to methods for treating homologous recombination (HR)-deficient cancer. The method comprises administering to the subject in need thereof a DNA polymerase θ (Polθ) inhibitor in an amount effective to treat the HR-deficient cancer.

As used herein, “homologous recombination (HR)”, refers to the cellular process of genetic recombination in which nucleotide sequences are exchanged between two similar or identical molecules of DNA. It is most widely used for repairing double-stranded breaks in DNA. Two primary models for how homologous recombination repairs double-strand breaks in DNA are the double-strand break repair (DSBR) pathway (sometimes called the double Holliday junction model) and the synthesis-dependent strand annealing (SDSA) pathway (See, e.g., Sung, P; Klein, H (October 2006). “Mechanism of homologous recombination: mediators and helicases take on regulatory functions”. Nature Reviews Molecular Cell Biology 7 (10): 739-750, incorporated herein by reference).

As used herein, “homologous recombination (HR)-deficient cancer” refers to a cancer characterized by a lack of a functional homologous recombination (HR) DNA repair pathway. Generally, HR-deficiency arises from a mutation or mutations in one or more HR-associated genes, such as BRCA1, BRCA2, RAD54, RAD51B, CtlP (Choline Transporter-Like Protein), PALB2 (Partner and Localizer of BRCA2), XRCC2 (X-ray repair complementing defective repair in Chinese hamster cells 2), RECQL4 (RecQ Protein-Like 4), BLM (Bloom syndrome, RecQ helicase-like), WRN (Werner syndrome, RecQ helicase-like), Nbs1 (Nibrin), and genes encoding Fanconi anemia (FA) proteins or FA-like genes. Examples of FA and FA-like genes include FANCA, FANCB, FANCC, FANCD1 (BRCA2), FANCD2, FANCE, FANCF, FANCG, FANCI, FANCJ (BRIP1), FANCL, FANCM, FANCN (PALB2), FANCP (SLX4), FANCS (BRCA1), RAD51C, and XPF.

Examples of cancers known to have mutations in HR-associated genes (and are, thus, HR-deficient cancers) include, but are not limited to, ovarian cancer, breast cancer, prostate cancer, non-Hodgkin's lymphoma, colon cancer, lipoma, uterine leiomyoma, basal cell skin carcinoma, squamous cell skin carcinoma, osteosarcoma, acute myelogenous leukemia (AML), and other cancers (See, e.g., Helleday (2010) Carcinigenesis vol. 21, no. 6, pp 955-960; D'Andrea A D. Susceptibility pathways in Fanconi's anemia and breast cancer. 2010 N Engl J Med. 362: 1909-1919).

In some embodiments, a HR-deficient cancer is breast cancer. Breast cancer includes, but is not limited to, lobular carcinoma in situ (LCIS), a ductal carcinoma in situ (DCIS), an invasive ductal carcinoma (IDC), inflammatory breast cancer, Paget disease of the nipple, Phyllodes tumor, Angiosarcoma, adenoid cystic carcinoma, low-grade adenosquamous carcinoma, medullary carcinoma, mucinous carcinoma, papillary carcinoma, tubular carcinoma, metaplastic carcinoma, micropapillary carcinoma, mixed carcinoma, or another breast cancer, including but not limited to triple negative, HER positive, estrogen receptor positive, progesterone receptor positive, HER and estrogen receptor positive, HER and progesterone receptor positive, estrogen and progesterone receptor positive, and HER and estrogen and progesterone receptor positive.

In some embodiments, a HR-deficient cancer is ovarian cancer. Ovarian cancer includes, but is not limited to, epithelial ovarian carcinomas (EOC), maturing teratomas, dysgerminomas, endodermal sinus tumors, granulosa-theca tumors, Sertoli-Leydig cell tumors, and primary peritoneal carcinoma.

The method involves administering to a subject in need thereof a DNA polymerase θ (Polθ) inhibitor. DNA polymerase θ (Polθ, also referred to as PolQ; Gene ID No. 10721) is a family A DNA polymerase that also functions as an DNA-dependent ATPase (see, eg., Seki et al. Nucl. Acids Res. (2003) 31 (21): 6117-6126). Polθ is implicated in a pathway required for the repair of double-stranded DNA breaks, referred to as the error-prone microhomology-mediated end-joining (MMEJ) pathway.

As used herein, a “Polθ inhibitor” (also referred to as a “PolQ inhibitor”) is any agent that reduces, slows, halts, and/or prevents Polθ activity in a cell relative to vehicle, or an agent that reduces or prevents expression of Polθ protein. Typically, Polθ comprises two distinct enzymatic (catalytic) domains, an N-terminal ATPase and a C-terminal polymerase domain. Thus, a Polθ inhibitor can be an agent (e.g., a small molecule, peptide or antisense molecule) that inhibits polymerase function, ATPase function, or polymerase function and ATPase function of Polθ. In some embodiments, the inhibitor reduces, slows, halts, and/or prevents the ATPase activity of Polθ. A Polθ inhibitor can be any molecule or compound that inhibits Polθ as described above, including a small molecule, antibody or antibody fragments, peptide or antisense compound, siRNA and shRNA, and DNA and RNA aptamers.

In some embodiments, a Polθ inhibitor is a molecule that reduces or prevents expression of Polθ, such as one or more antisense molecules (e.g., siRNA, shRNA, dsRNA, miRNA, amiRNA, antisense oligonucleotides (ASO)) that target DNA or mRNA encoding Polθ. In some embodiments, the antisense molecule is an interfering RNA (e.g., dsRNA, siRNA, shRNA, miRNA, amiRNA, ASO). In some embodiments, a Polθ inhibitor is an interfering RNA having a sequence as set forth in SEQ ID NO: 6. The skilled artisan recognizes that antisense compounds can be unmodified or modified. Modified antisense compounds may comprise modified nucleobases, modified sugars, modified backbones, or any combination of the foregoing modifications. Examples of modifications include, but are not limited to 2′O-Me modifications, 2′-F modification, substitution of unlocked nucleobase analogs, and phosphorothioate backbone modification.

A “subject in need of treatment” is a subject identified as having a homologous recombination (HR)-deficient cancer, i.e., the subject has been diagnosed by a physician (e.g., using methods well known in the art; see WO 2014/138101, incorporated herein by reference) as having a HR-deficient cancer. The HR status of the cancer can be determined by, for example, a BRCA 1-specific CGH classifier (Evers et al. Trends Pharmacol Sci. 2010 August; 31(8):372-80), an assay that determines the capacity of primary cell cultures to form RAD51 foci after PARP inhibition (Mukhopadhyay, A. et al. (2010) Clin. Cancer Res. 16, 2344-2351), or determining the methylation status of BRACA1 (and other HR-associated genes) (Evers et al. Trends Pharmacol Sci. 2010 August; 31(8):372-80). In some embodiments, the HR-deficient cancer is resistant to treatment with a poly (ADP-ribose) polymerase (PARP) inhibitor alone (see, for example, Montoni et al. Front Pharmacol. 2013 Feb. 27; 4:18).

PARP is an enzyme that plays a critical role in DNA repair and recently, alterations or changes in DNA repair pathways have been implicated in the pathogenesis of some human cancers. Consequently, PARP inhibition has been put forward as a potential strategy to treat human cancers. Several small molecule inhibitors of PARP activity have been developed and brought forward into clinical development. Some have shown growth inhibitory activity in a small but distinct number of human cancer cell lines and patient tumors that lack specific DNA repair mechanisms either through inherited mutations and/or non-inherited silencing of genes such as, but not limited to, BRCA-1 and 2. Other known genes encoding proteins critical to DNA repair functions have also been implicated as mutation targets in the malignant process of some cancers.

As used herein, the term “PARP” includes at least PARP1 and PARP2. PARP1 is the founding member of a large family of poly(ADP-ribose) polymerases with 17 members identified (Ame et ah, Bioessays 26:882-893, 2004). It is the primary enzyme catalyzing the transfer of ADP-ribose units from NAD+ to target proteins including PARP1 itself. Under normal physiologic conditions, PARP1 facilitates the repair of DNA base lesions by helping recruit base excision repair proteins XRCC1 and Poιβ (Dantzer et ah, Methods Enzymol. 409:493-510, 2006).

Typically, PARP expression and activity are significantly up-regulated in certain cancers, suggesting that these cancer cells may rely more than normal cells on the activity of PARP. Thus, agents that inhibit the activity of PARP or reduce the expression level of PARP, collectively referred to herein as “PARP inhibitors (PARPi)”, may be useful cancer therapeutics. Examples of PARPi include, but are not limited to, iniparib (BSI 201), talazoparib (BMN-673), olaparib (AZD-2281, TOPARP-A), rucaparib (AG014699, PF-01367338), veliparib (ABT-888), CEP 9722, MK 4827, BGB-290 and 3-aminobenzamide, 4-amino-1,8-napthalimide, benzamide, BGP-15, BYK204165, 3,4-Dihydro-5-[4-(1-piperidinyl)butoxyl]-1(2H)-isoquinolinone, DR2313, 1,5-Isoquinolinediol, MC2050, ME0328, PJ-34 hydrochloride hydrate, and UPF-1069.

It has been found, in accordance with the invention, that POLQ channels HR repair by antagonizing HR and promoting poly (ADP-ribose) polymerase (PARP)-dependent error-prone repair. Without wishing to be bound by any particular theory, inhibition of POLQ is expected to enhance cell death of PARP inhibitor-resistant cancers. For instance, the PARP enzyme cooperates with POLQ in the process of Alternative End-Joining Repair (Alt-EJ). PARP is required to localize POLQ at the site of the double strand break (dsb) repair). Human tumors can become resistant to PARP inhibitors; however, these tumors may still be sensitive to a POLQ inhibitor if POLQ can localize to the dsb in a PARP-independent manner. Accordingly, aspects of the disclosure provide methods for treating a cancer that is resistant to poly (ADP-ribose) polymerase (PARP) inhibitor therapy in a subject. The method comprises administering to the subject in need thereof a DNA polymerase θ (Polθ) inhibitor in an amount effective to treat the PARP inhibitor-resistant cancer.

As used herein, a cancer that is resistant to a PARP inhibitor means that the cancer does not respond to such inhibitor, for example as evidenced by continued proliferation and increasing tumor growth and burden. In some instances, the cancer may have initially responded to treatment with such inhibitor (referred to herein as a previously administered therapy) but may have grown resistant after a time. In some instances, the cancer may have never responded to treatment with such inhibitor at all. Cancers resistant to PARP inhibitors can be identified using methods known in the art (see, e.g., WO 2014205105, U.S. Pat. No. 8,729,048; incorporated herein by reference). Examples of cancers resistant to PARP-inhibitors include, but are not limited to, breast cancer, ovarian cancer, lung cancer, bladder cancer, liver cancer, head and neck cancer, pancreatic cancer, gastrointestinal cancer, and colorectal cancer.

Aspects of the disclosure involve administering a POLQ inhibitor for treating PARP inhibitor-resistant cancers. POLQ inhibitors have been described herein, and include any agent that reduces, slows, halts, and/or prevents Polθ activity, including a small molecule, antibody or antibody fragments, peptide or antisense compound, siRNA and shRNA, and DNA and RNA aptamers.

A “subject in need of treatment” is a subject identified as having a cancer that is resistant to or at risk of developing resistance to PARP inhibitor therapy using methods well known in the art (see, e.g., WO 2014205105, WO 2015040378, WO 2011153345; incorporated herein by reference). In some embodiments, the PARP inhibitor-resistant cancer is deficient in homologous recombination (i.e., the cancer is characterized by a lack of a functional homologous recombination (HR) DNA repair pathway, and is resistant to PARP inhibitor therapy).

The inventors have also recognized and appreciated that Polθ expression is up-regulated in certain cancers (e.g., HR-deficient cancers). Thus, in some aspects, the disclosure provides a method for treating a cancer that is characterized by overexpression of DNA polymerase θ (Polθ) in a subject, the method comprising: administering to the subject in need thereof a DNA polymerase θ (Polθ) inhibitor in an amount effective to treat the Polθ-overexpressing cancer.

The term “Polθ overexpressing cancer” refers to the increased expression or activity of Polθ in a cancerous cell relative to expression or activity of Polθ in a control cell (e.g., a non-cancerous cell of the same type). The amount of Polθ overexpression can be at least 2-fold, at least 3-fold, at least 4-fold, at least 5-fold, at least 10-fold, at least 20-fold, at least 50-fold, at least 100-fold, at least 500-fold, or at least 1000-fold relative to Polθ expression in a control cell. In some embodiments, Polθ overexpression ranges from about 2-fold to about 500-fold compared to a control sample. Examples of Polθ overexpressing cancers include, but are not limited to, certain ovarian, breast, cervical, lung, colorectal, gastric, bladder, and prostate cancers.

Aspects of the disclosure involve administering a POLQ inhibitor for treating POLQ overexpressing cancers. POLQ inhibitors have been described herein, and include any agent that reduces, slows, halts, and/or prevents POLQ activity, including a small molecule, antibody or antibody fragments, peptide or antisense compound, siRNA and shRNA, and DNA and RNA aptamers.

A “subject in need of treatment” is a subject identified as having a POLQ overexpressing cancer using methods well known in the art (see, e.g., EP 2710142; incorporated by reference herein). The POLQ status of the cancer can be determined, for example, by measuring the level of mRNA and/or protein using methods known in the art, such as but not limited to, Northern blot, quantitative PCR, nucleic acid microarray technologies, Western blot, ELISA or ELISPOT, antibodies microarrays, or immunohistochemistry. In some embodiments, the POLQ overexpressing cancer is deficient in homologous recombination (i.e., the cancer is characterized by a lack of a functional homologous recombination (HR) DNA repair pathway, and overexpresses POLQ).

It has been found, in accordance with the invention, that an inverse relationship exists between homologous recombination (HR) activity and DNA polymerase θ (Polθ) expression. Knockdown of Polθ was, surprisingly, found to enhance cell death in HR-deficient cancers. Consistent with these results, genetic inactivation of an HR gene (Fancd2) and Polθ in mice was found to result in embryonic lethality. HR-deficient cancers lack of a functional homologous recombination (HR) DNA repair pathway, and typically arise due to one or more mutations in one or more HR-associated genes, such as BRCA1, BRCA2, and genes encoding Fanconi anemia (FA) proteins or FA-like genes. Without wishing to be bound by any particular theory, inhibition of POLQ is expected to enhance cell death of cancers that are characterized by one or more BRCA mutations and/or reduced expression of Fanconi (Fanc) proteins.

Accordingly, aspects of the disclosure provide a method for treating a cancer that is characterized by one or more BRCA mutations and/or reduced expression of Fanconi (Fanc) proteins in a subject. The method comprises administering to the subject in need thereof a DNA polymerase θ (Polθ) inhibitor in an amount effective to treat the cancer. In some embodiments, the cancer characterized by one or more BRCA mutations and/or reduced expression of Fanconi (Fanc) proteins is also characterized by overexpression of DNA polymerase θ (Polθ).

Genetic susceptibility to breast cancer has been linked to mutations of the BRCA1 and BRCA2 genes. It is postulated that a mutation causes a disruption in the protein which causes chromosomal instability in BRCA deficient cells thereby predisposing them to neoplastic transformation. Inherited mutations in the BRCA1 and BRCA2 genes account for approximately 7-10% of all breast cancer cases. Women with BRCA mutations have a lifetime risk of breast cancer between 56-87%, and a lifetime risk of ovarian cancer between 27-44%. In addition, mutations in BRCA genes have also been linked to various other tumors including, e.g., pancreatic cancer. As used herein, a BRCA mutation is a mutation in either of the BRCA1 and BRCA2 genes, and which leads to cancer in affected persons.

Located on chromosome 17, BRCA1 is the first gene identified conferring increased risk for breast and ovarian cancer (Miki et al., Science, 266:66-71 (1994)). The BRCA1 gene (Gene ID: 672) is divided into 24 separate exons. Exons 1 and 4 are noncoding, in that they are not part of the final functional BRCA1 protein product. The BRCA1 coding region spans roughly 5600 base pairs (bp). Each exon consists of 200-400 bp, except for exon 11 which contains about 3600 bp.

Wooster et al. (Nature 378: 789-792, 1995) identified the BRCA2 gene by positional cloning of a region on chromosome 13q12-q13 implicated in Icelandic families with breast cancer. Human BRCA2 (Gene ID: 675) gene contains 27 exons. Similar to BRCA1, BRCA2 gene also has a large exon 11, translational start sites in exon 2, and coding sequences that are AT-rich.

Mutations of BRCA genes associated with cancer (i.e., predisposing the subject to developing cancer) are well known in the art (see, e.g., Friend, S. et al., 1995, Nature Genetics 11: 238, US 2003/0235819, U.S. Pat. No. 6,083,698, U.S. Pat. No. 7,250,497, U.S. Pat. No. 5,747,282, WO 1999028506, U.S. Pat. No. 5,837,492, WO 2014160876; incorporated herein by reference). Methods to identify BRCA mutations are known in the art (see, for example, WO1998043092, WO 2013124740; incorporated herein by reference).

In some embodiments, the cancer is characterized by reduced expression of one or more Fanconi (Fanc) proteins in a subject. “Reduced expression of one or more Fanconi (Fanc) proteins” refers to the reduced expression of one or more Fanconi (Fanc) proteins in a cancerous cell relative to expression of the protein(s) in a control cell (e.g., a non-cancerous cell of the same type). The expression of the protein(s) may be reduced by at least 2-fold, at least 3-fold, at least 4-fold, at least 5-fold, at least 10-fold, at least 20-fold, at least 50-fold, at least 100-fold, at least 500-fold, or at least 1000-fold relative to the expression in a control cell. In some embodiments, the expression of the protein(s) may be reduced by about 2-fold to about 500-fold compared to a control sample. Examples of FA and FA-like genes include FANCA, FANCB, FANCC, FANCD1 (BRCA2), FANCD2, FANCE, FANCF, FANCG, FANCI, FANCJ (BRIP1), FANCL, FANCM, FANCN (PALB2), FANCP (SLX4), FANCS (BRCA1), RAD51C, and XPF. Examples of cancers that are characterized by reduced expression of one or more Fanconi (Fanc) proteins include, but are not limited to, certain ovarian, breast, cervical, lung, colorectal, gastric, bladder, and prostate cancers.

Aspects of the disclosure involve administering a POLQ inhibitor for treating cancer that is characterized by one or more BRCA mutations and/or reduced expression of Fanconi (Fanc) proteins in a subject. POLQ inhibitors have been described herein, and include any agent that reduces, slows, halts, and/or prevents POLQ activity, including a small molecule, antibody or antibody fragments, peptide or antisense compound, siRNA and shRNA, and DNA and RNA aptamers.

A “subject in need of treatment” is a subject identified as having a cancer that is characterized by one or more BRCA mutations and/or reduced expression of Fanconi (Fanc) proteins in a subject. The mutational status of the BRCA proteins can be determined using assays known in the art (see, for example, WO1998043092, WO 2013124740; incorporated herein by reference). The expression status of the one or more Fanconi proteins can be determined, for example, by measuring the level of mRNA and/or protein using methods known in the art, such as but not limited to, Northern blot, quantitative PCR, nucleic acid microarray technologies, Western blot, ELISA or ELISPOT, antibodies microarrays, or immunohistochemistry. In some embodiments, the cancer is also characterized by overexpression of POLQ (i.e., the cancer is characterized by one or more BRCA mutations and/or reduced expression of Fanconi (Fanc) proteins, and overexpresses POLQ).

### Anti-Cancer Therapies

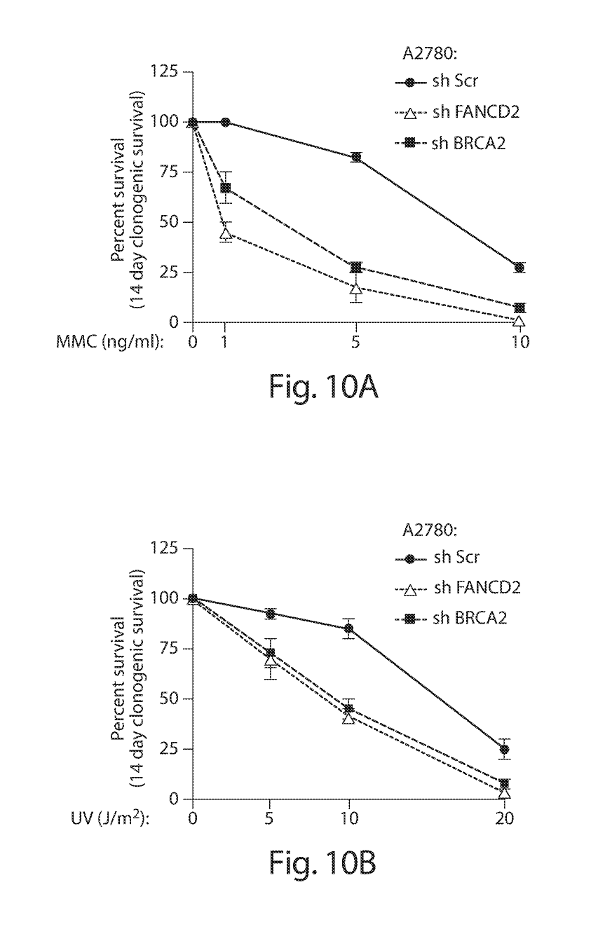

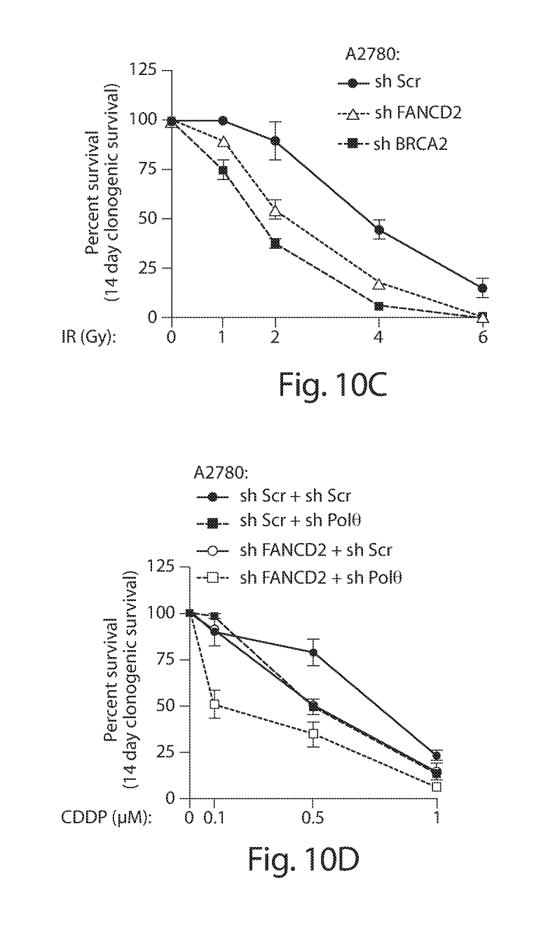

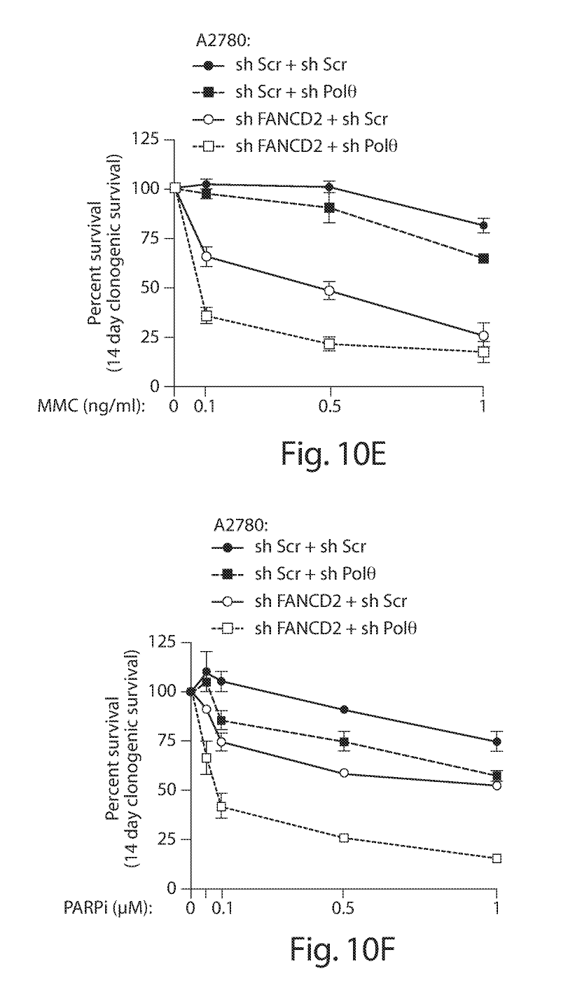

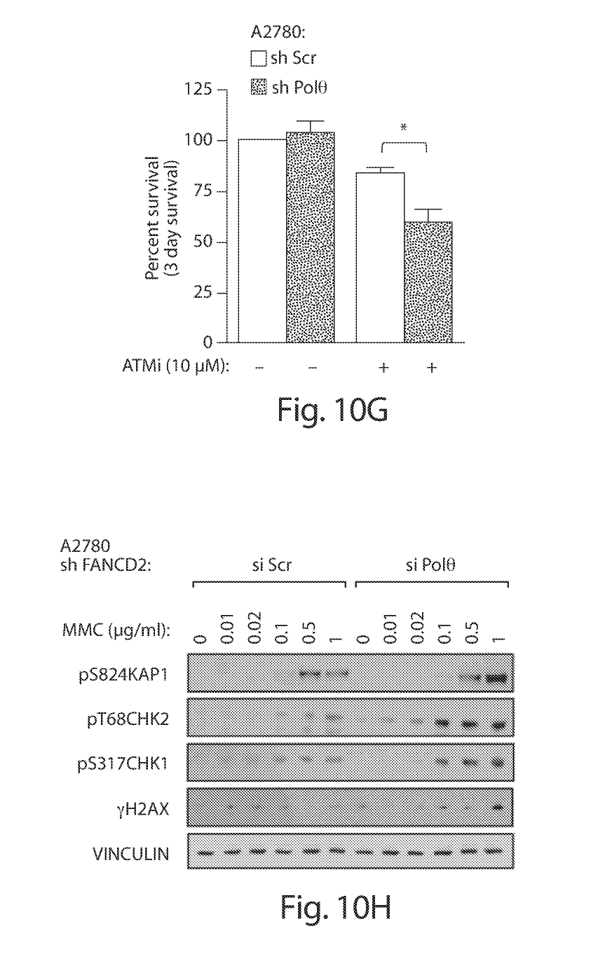

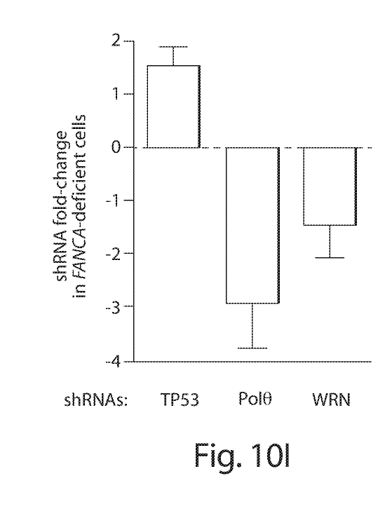

Some aspects of the disclosure relate, in part, to the discovery that Polθ inhibitors and anti-cancer therapies (e.g., anti-cancer agents, or therapies such as surgery, transplantation or radiotherapy) show a synergistic effect in the treatment of cancers described herein (e.g., HR-deficient cancers, cancers resistant to poly (ADP-ribose) polymerase (PARP) inhibitor therapy, POLQ overexpressing cancer, and/or cancers characterized by one or more BRCA mutations and/or reduced expression of Fanconi (Fanc) proteins). As used herein, “synergistic” refers to the joint action of agents (e.g., pharmaceutically active agents), that when taken together increase each other's effectiveness. The synergistic effects of Polθ inhibitor/anti-cancer therapy combinations are described in the Examples section and in FIGS. 10A-10I.

Accordingly, the methods described herein further comprise treating a subject with one or more anti-cancer therapy. As used herein, “anti-cancer therapy” refers to any agent, composition or medical technique (e.g., surgery, radiation treatment, etc.) useful for the treatment of cancer. For example, an anti-cancer agent can be a small molecule, antibody, peptide or antisense compound. Examples of antisense compounds include, but are not limited to interfering RNAs (e.g., dsRNA, siRNA, shRNA, miRNA, and amiRNA) and antisense oligonucleotides (ASO).

In some embodiments, the anti-cancer therapy is selected from the group consisting of surgery, radiation therapy, chemotherapy, gene therapy, DNA therapy, viral therapy, RNA therapy, adjuvant therapy, and immunotherapy.

In some embodiments, the chemotherapy comprises administering to the subject a cytotoxic agent in an amount effective to treat the HR-deficient cancer. In some embodiments, the cytotoxic agent is selected from the group consisting of a platinum agent, mitomycin C, a poly (ADP-ribose) polymerase (PARP) inhibitor, a radioisotope, a vinca alkaloid, an antitumor alkylating agent, a monoclonal antibody and an antimetabolite. In some embodiments, the cytotoxic agent is an ataxia telangiectasia mutated (ATM) kinase inhibitor.

Examples of platinum agents include, but are not limited to cisplatin, carboplatin, oxaliplatin, satraplatin, picoplatin, Nedaplatin, Triplatin, and Lipoplatin.

Examples of cytotoxic radioisotopes include but are not limited to ⁶⁷Cu, ⁶⁷Ga, ⁹⁰Y, ¹³¹I, ¹⁷⁷Lu, ¹⁸⁶Re, ¹⁸⁸Re, α-Particle emitter, ²¹¹At, ²¹³Bi, ²²⁵Ac, Auger-electron emitter, ¹²⁵I, ²¹²Pb, and ¹¹¹In.

Examples of antitumor alkylating agents include, but are not limited to nitrogen mustards, cyclophosphamide, mechlorethamine or mustine (HN2), uramustine or uracil mustard, melphalan, chlorambucil, ifosfamide, bendamustine, nitrosoureas, carmustine, lomustine, streptozocin, alkyl sulfonates, busulfan, thiotepa, procarbazine, altretamine, triazenes, dacarbazine, mitozolomide, and temozolomide.

Examples of anti-cancer monoclonal antibodies include, but are not limited to necitumumab, dinutuximab, nivolumab, blinatumomab, pembrolizumab, ramucirumab, obinutuzumab, adotrastuzumab emtansine, pertuzumab, brentuximab, ipilimumab, ofatumumab, catumaxomab, bevacizumab, cetuximab, tositumomab-I131, ibritumomab tiuxetan, alemtuzumab, gemtuzumab ozogamicin, trastuzumab, and rituximab.

Examples of vinca alkaloids include, but are not limited to vinblastine, vincristine, vindesine, vinorelbine, desoxyvincaminol, vincaminol, vinburnine, vincamajine, vineridine, vinburnine, and vinpocetine.

Examples of antimetabolites include, but are not limited to fluorouracil, cladribine, capecitabine, mercaptopurine, pemetrexed, fludarabine, gemcitabine, hydroxyurea, methotrexate, nelarbine, clofarabine, cytarabine, decitabine, pralatrexate, floxuridine, and thioguanine.

In some embodiments, the anti-cancer therapy is an immunotherapy, such as, but not limited to, cellular immunotherapy, antibody therapy or cytokine therapy. Without wishing to be bound by any particular theory, POLQ inhibitors are expected to function in many ways similar to PARP inhibitors, and to synergize with immunotherapy. Examples of cellular immunotherapy include, but is not limited to, dendritic cell therapy and Sipuleucel-T. Examples of antibody therapy include, but is not limited to Alemtuzumab, Ipilimumab, Nivolumab, Ofatumumab, Pembrolizumab, and Rituximab. Examples of cytokine therapy include, but is not limited to, interferons (for example, IFNα, IFNβ, IFNγ, IFNλ) and interleukins. In some embodiments, the immunotherapy comprises one or more immune checkpoint inhibitors. Examples of immune checkpoint proteins include, but are not limited to, CTLA-4 and its ligands CD80 and CD86, PD-1 with its ligands PD-L1 and PD-L2, and 4-1BB.

Additional examples of anti-cancer therapies include, but are not limited to, abiraterone acetate (e.g., ZYTIGA), ABVD, ABVE, ABVE-PC, AC, AC-T, ADE, ado-trastuzumab emtansine (e.g., KADCYLA), afatinib dimaleate (e.g., GILOTRIF), aldesleukin (e.g., PROLEUKIN), alemtuzumab (e.g., CAMPATH), anastrozole (e.g., ARIMIDEX), arsenic trioxide (e.g., TRISENOX), asparaginase erwinia chrysanthemi (e.g., ERWINAZE), axitinib (e.g., INLYTA), azacitidine (e.g., MYLOSAR, VIDAZA), BEACOPP, belinostat (e.g., BELEODAQ), bendamustine hydrochloride (e.g., TREANDA), BEP, bevacizumab (e.g., AVASTIN), bicalutamide (e.g., CASODEX), bleomycin (e.g., BLENOXANE), blinatumomab (e.g., BLINCYTO), bortezomib (e.g., VELCADE), bosutinib (e.g., BOSULIF), brentuximab vedotin (e.g., ADCETRIS), busulfan (e.g., BUSULFEX, MYLERAN), cabazitaxel (e.g., JEVTANA), cabozantinib-s-malate (e.g., COMETRIQ), CAF, capecitabine (e.g., XELODA), CAPDX, carboplatin (e.g., PARAPLAT, PARAPLATIN), carboplatin-taxol, carfilzomib (e.g., KYPROLIS), carmustine (e.g., BECENUM, BICNU, CARMUBRIS), carmustine implant (e.g., GLIADEL WAFER, GLIADEL), ceritinib (e.g., ZYKADIA), cetuximab (e.g., ERBITUX), chlorambucil (e.g., AMBOCHLORIN, AMBOCLORIN, LEUKERAN, LINFOLIZIN), chlorambucil-prednisone, CHOP, cisplatin (e.g., PLATINOL, PLATINOL-AQ), clofarabine (e.g., CLOFAREX, CLOLAR), CMF, COPP, COPP-ABV, crizotinib (e.g., XALKORI), CVP, cyclophosphamide (e.g., CLAFEN, CYTOXAN, NEOSAR), cytarabine (e.g., CYTOSAR-U, TARABINE PFS), dabrafenib (e.g., TAFINLAR), dacarbazine (e.g., DTIC-DOME), dactinomycin (e.g., COSMEGEN), dasatinib (e.g., SPRYCEL), daunorubicin hydrochloride (e.g., CERUBIDINE), decitabine (e.g., DACOGEN), degarelix, denileukin diftitox (e.g., ONTAK), denosumab (e.g., PROLIA, XGEVA), Dinutuximab (e.g., UNITUXIN), docetaxel (e.g., TAXOTERE), doxorubicin hydrochloride (e.g., ADRIAMYCIN PFS, ADRIAMYCIN RDF), doxorubicin hydrochloride liposome (e.g., DOXIL, DOX-SL, EVACET, LIPODOX), enzalutamide (e.g., XTANDI), epirubicin hydrochloride (e.g., ELLENCE), EPOCH, erlotinib hydrochloride (e.g., TARCEVA), etoposide (e.g., TOPOSAR, VEPESID), etoposide phosphate (e.g., ETOPOPHOS), everolimus (e.g., AFINITOR DISPERZ, AFINITOR), exemestane (e.g., AROMASIN), FEC, fludarabine phosphate (e.g., FLUDARA), fluorouracil (e.g., ADRUCIL, EFUDEX, FLUOROPLEX), FOLFIRI, FOLFIRI-BEVACIZUMAB, FOLFIRI-CETUXIMAB, FOLFIRINOX, FOLFOX, FU-LV, fulvestrant (e.g., FASLODEX), gefitinib (e.g., IRESSA), gemcitabine hydrochloride (e.g., GEMZAR), gemcitabine-cisplatin, gemcitabine-oxaliplatin, goserelin acetate (e.g., ZOLADEX), Hyper-CVAD, ibritumomab tiuxetan (e.g., ZEVALIN), ibrutinib (e.g., IMBRUVICA), ICE, idelalisib (e.g., ZYDELIG), ifosfamide (e.g., CYFOS, IFEX, IFOSFAMIDUM), imatinib mesylate (e.g., GLEEVEC), imiquimod (e.g., ALDARA), ipilimumab (e.g., YERVOY), irinotecan hydrochloride (e.g., CAMPTOSAR), ixabepilone (e.g., IXEMPRA), lanreotide acetate (e.g., SOMATULINE DEPOT), lapatinib ditosylate (e.g., TYKERB), lenalidomide (e.g., REVLIMID), lenvatinib (e.g., LENVIMA), letrozole (e.g., FEMARA), leucovorin calcium (e.g., WELLCOVORIN), leuprolide acetate (e.g., LUPRON DEPOT, LUPRON DEPOT-3 MONTH, LUPRON DEPOT-4 MONTH, LUPRON DEPOT-PED, LUPRON, VIADUR), liposomal cytarabine (e.g., DEPOCYT), lomustine (e.g., CEENU), mechlorethamine hydrochloride (e.g., MUSTARGEN), megestrol acetate (e.g., MEGACE), mercaptopurine (e.g., PURINETHOL, PURIXAN), methotrexate (e.g., ABITREXATE, FOLEX PFS, FOLEX, METHOTREXATE LPF, MEXATE, MEXATE-AQ), mitomycin c (e.g., MITOZYTREX, MUTAMYCIN), mitoxantrone hydrochloride, MOPP, nelarabine (e.g., ARRANON), nilotinib (e.g., TASIGNA), nivolumab (e.g., OPDIVO), obinutuzumab (e.g., GAZYVA), OEPA, ofatumumab (e.g., ARZERRA), OFF, olaparib (e.g., LYNPARZA), omacetaxine mepesuccinate (e.g., SYNRIBO), OPPA, oxaliplatin (e.g., ELOXATIN), paclitaxel (e.g., TAXOL), paclitaxel albumin-stabilized nanoparticle formulation (e.g., ABRAXANE), PAD, palbociclib (e.g., IBRANCE), pamidronate disodium (e.g., AREDIA), panitumumab (e.g., VECTIBIX), panobinostat (e.g., FARYDAK), pazopanib hydrochloride (e.g., VOTRIENT), pegaspargase (e.g., ONCASPAR), peginterferon alfa-2b (e.g., PEG-INTRON), peginterferon alfa-2b (e.g., SYLATRON), pembrolizumab (e.g., KEYTRUDA), pemetrexed disodium (e.g., ALIMTA), pertuzumab (e.g., PERJETA), plerixafor (e.g., MOZOBIL), pomalidomide (e.g., POMALYST), ponatinib hydrochloride (e.g., ICLUSIG), pralatrexate (e.g., FOLOTYN), prednisone, procarbazine hydrochloride (e.g., MATULANE), radium 223 dichloride (e.g., XOFIGO), raloxifene hydrochloride (e.g., EVISTA, KEOXIFENE), ramucirumab (e.g., CYRAMZA), R-CHOP, recombinant HPV bivalent vaccine (e.g., CERVARIX), recombinant human papillomavirus (e.g., HPV) nonavalent vaccine (e.g., GARDASIL 9), recombinant human papillomavirus (e.g., HPV) quadrivalent vaccine (e.g., GARDASIL), recombinant interferon alfa-2b (e.g., INTRON A), regorafenib (e.g., STIVARGA), rituximab (e.g., RITUXAN), romidepsin (e.g., ISTODAX), ruxolitinib phosphate (e.g., JAKAFI), siltuximab (e.g., SYLVANT), sipuleucel-t (e.g., PROVENGE), sorafenib tosylate (e.g., NEXAVAR), STANFORD V, sunitinib malate (e.g., SUTENT), TAC, tamoxifen citrate (e.g., NOLVADEX, NOVALDEX), temozolomide (e.g., METHAZOLASTONE, TEMODAR), temsirolimus (e.g., TORISEL), thalidomide (e.g., SYNOVIR, THALOMID), thiotepa, topotecan hydrochloride (e.g., HYCAMTIN), toremifene (e.g., FARESTON), tositumomab and iodine I 131 tositumomab (e.g., BEXXAR), TPF, trametinib (e.g., MEKINIST), trastuzumab (e.g., HERCEPTIN), VAMP, vandetanib (e.g., CAPRELSA), VEIP, vemurafenib (e.g., ZELBORAF), vinblastine sulfate (e.g., VELBAN, VELSAR), vincristine sulfate (e.g., VINCASAR PFS), vincristine sulfate liposome (e.g., MARQIBO), vinorelbine tartrate (e.g., NAVELBINE), vismodegib (e.g., ERIVEDGE), vorinostat (e.g., ZOLINZA), XELIRI, XELOX, ziv-aflibercept (e.g., ZALTRAP), zoledronic acid (e.g., ZOMETA), or a combination thereof. In certain embodiments, the anti-cancer therapy is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors, modulators of protein stability (e.g., proteasome inhibitors), Hsp90 inhibitors, glucocorticoids, all-trans retinoic acids, and other agents that promote differentiation. In certain embodiments, a Polθ inhibitor can be independently administered in combination with an anti-cancer therapy including, but not limited to, surgery, radiation therapy, transplantation (e.g., stem cell transplantation, bone marrow transplantation), immunotherapy, and chemotherapy.

Additional examples of cancers that may be treated using the methods described herein include, but are not limited to, lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms' tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The terms “treatment,” “treat,” and “treating” refer to reversing, alleviating, delaying the onset of, or inhibiting the progress of cancer. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence.

The terms “administer,” “administering,” or “administration” refers to implanting, absorbing, ingesting, injecting, inhaling, or otherwise introducing a compound described herein, or a composition thereof, in or on a subject.

The terms “inhibition”, “inhibiting”, “inhibit,” or “inhibitor” refer to the ability of a compound to reduce, slow, halt, and/or prevent activity of a particular biological process in a cell relative to vehicle. In some embodiments, “inhibit”, “block”, “suppress” or “prevent” means that the activity being inhibited, blocked, suppressed, or prevented is reduced by at least 5%, 10%, 15%, 20%, 25%, 30%, 35%, 40%, 45%, 50%, 55%, 60%, 65%, 70%, 75%, 80%, 85%, 90%, 95%, or 100% as compared to the activity of a control (e.g., activity in the absence of the inhibitor). In some embodiments, “inhibit”, “block”, “suppress” or “prevent” means that the expression of the target of the inhibitor (e.g. POLQ) is reduced by at least 5%, 10%, 15%, 20%, 25%, 30%, 35%, 40%, 45%, 50%, 55%, 60%, 65%, 70%, 75%, 80%, 85%, 90%, 95%, 100% as compared to a control (e.g., the expression in the absence of the inhibitor). In some embodiments, “inhibit”, “block”, “suppress” or “prevent” means that the activity of the target of the inhibitor (e.g. the ATPase activity of POLQ) is reduced by at least 5%, 10%, 15%, 20%, 25%, 30%, 35%, 40%, 45%, 50%, 55%, 60%, 65%, 70%, 75%, 80%, 85%, 90%, 95%, 100% as compared to a control (e.g., the ATPase activity of POLQ in the absence of the inhibitor).

An “effective amount” refers to an amount sufficient to elicit the desired biological response, i.e., treating cancer. As will be appreciated by those of ordinary skill in this art, the effective amount of the compounds described herein may vary depending on such factors as the desired biological endpoint, the pharmacokinetics of the compound, the condition being treated, the mode of administration, and the age and health of the subject. An effective amount includes, but is not limited to, that amount necessary to slow, reduce, inhibit, ameliorate or reverse one or more symptoms associated with cancer. For example, in the treatment of cancer, such terms may refer to a reduction in the size of the tumor.

In some embodiments, an effective amount is an amount of agent (e.g., Pol0 inhibitor) that results in a reduction of Polθ expression and/or activity in the cancer cells. The reduction in Polθ expression and/or activity resulting from administration of an effective amount of Polθ inhibitor can range from about 2-fold to about 500-fold, 5-fold to about 250-fold, 10-fold to about 150-fold, or about 20-fold to about 100-fold. In some embodiments, reduction in Polθ expression and/or activity resulting from administration of an effective amount of Polθ inhibitor can range from about 100% to about 1%, about 90% to about 10%, about 80% to about 20%, about 70% to about 30%, about 60% to about 40%. In some embodiments, an amount effective to treat the cancer results in a cell lacking expression and/or activity of Polθ (e.g., complete silencing or knockout of POLQ gene).

Where two or more inhibitors are administered to the subject, the effective amount may be a combined effective amount. The effective amount of a first inhibitor may be different when it is used with a second and optionally a third inhibitor. When two or more inhibitors are used together, the effective amounts of each may be the same as when they are used alone.

Alternatively, the effective amounts of each may be less than the effective amounts when they are used alone because the desired effect is achieved at lower doses. Alternatively, again, the effective amount of each may be greater than the effective amounts when they are used alone because the subject is better able to tolerate one or more of the inhibitors which can then be administered at a higher dose provided such higher dose provides more therapeutic benefit.

An effective amount of a compound may vary from about 0.001 mg/kg to about 1000 mg/kg in one or more dose administrations, for one or several days (depending on the mode of administration). In certain embodiments, the effective amount varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg. One of ordinary skill in the art would be able to determine empirically an appropriate therapeutically effective amount.

As used throughout, the term “subject” or “patient” is intended to include humans and animals that are capable of suffering from or afflicted with a cancer or any disorder involving, directly or indirectly, a cancer. Examples of subjects include mammals, e.g., humans, dogs, cows, horses, pigs, sheep, goats, cats, mice, rabbits, rats, and transgenic non-human animals. In some embodiments, subjects include companion animals, e.g. dogs, cats, rabbits, and rats. In some embodiments, subjects include livestock, e.g., cows, pigs, sheep, goats, and rabbits. In some embodiments, subjects include thoroughbred or show animals, e.g. horses, pigs, cows, and rabbits. In important embodiments, the subject is a human, e.g., a human having, at risk of having, or potentially capable of having cancer.

The compounds described herein can be administered to the subject in any order. A first therapeutic agent, such as POLQ inhibitor, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, such as an anti-cancer therapy described herein, to a subject with cancer. Thus, POLQ inhibitors can be administered separately, sequentially or simultaneously with the second therapeutic agent, such as a chemotherapeutic agent described herein.

The compounds described herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound, mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form. In certain embodiments, the compounds provided herein may be administered at dosage levels sufficient to deliver from about 0.001 mg/kg to about 100 mg/kg, from about 0.01 mg/kg to about 50 mg/kg, preferably from about 0.1 mg/kg to about 40 mg kg, preferably from about 0.5 mg kg to about 30 mg/kg, from about 0.01 mg kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

### Screening Methods

Methods of identifying Polθ inhibitors are also contemplated by the disclosure. In some aspects, the disclosure provides a high-throughput screening method for identifying an inhibitor of ATPase activity of DNA polymerase θ (Polθ), the method comprising: contacting Polθ or a fragment thereof with adenosine triphosphate (ATP) and single-stranded DNA (ssDNA) substrate in the presence and absence of a candidate compound; quantifying amount of adenosine diphosphate (ADP) produced in the presence and absence of the candidate compound; and, identifying the candidate compound as an inhibitor of the ATPase activity of Polθ if the amount of ADP produced in the presence of the candidate compound is less than the amount produced in the absence of candidate compound.

As described elsewhere in the disclosure, “an inhibitor of ATPase activity of Polθ” refers to an agent that reduces, slows, halts, and/or prevents Polθ ATPase activity in a cell relative to vehicle, or an agent that reduces or prevents expression of Polθ protein (such that the ATPase activity of Polθ is abrogated). An inhibitor of Polθ ATPase activity can be a small molecule, antibody, peptide, or antisense compound (e.g., an interfering RNA). In some embodiments, an inhibitor of Polθ ATPase activity targets the N-terminal ATPase domain of a Polθ protein.

The term “Polθ or a fragment thereof” refers to full-length Polθ protein (e.g., Pol0 protein comprising both an N-terminal ATPase domain and a C-terminal polymerase domain), a portion of a Polθ protein sufficient to catalyze ATP hydrolysis, or a portion of Polθ protein sufficient to function as a polymerase. In some embodiments, Polθ or fragment thereof comprises the N-terminal ATPase domain.

A “single-stranded DNA (ssDNA) substrate” is generated as described in Yusufzai, T. & Kadonaga, J. T. HARP is an ATP-driven annealing helicase Science 322, 748-750 (2008); incorporated by reference herein. In some embodiments, the ssDNA is 5′-GTTAGCAGGTACCGAGCAACAATTCACTGG-3′ (SEQ ID NO: 74).

A “candidate compound” refers to any compound wherein the characterization of the compound's ability to inhibit Polθ ATPase activity is desirable. In some embodiments, methods described by the disclosure are useful for screening large libraries of candidate compounds to identify new drugs that inhibit the ATPase activity of Polθ. Exemplary candidate agents include, but are not limited to small molecules, antibodies, antibody conjugates, peptides, proteins, and/or antisense molecules (e.g., interfering RNAs).

The skilled artisan recognizes several methods for contacting the Polθ or portion thereof with the candidate compound. For example, automated liquid handling systems are generally utilized for high throughput drug screening. Automated liquid handling systems utilize arrays of liquid dispensing vessels, controlled by a robotic arm, to distribute fixed volumes of liquid to the wells of an assay plate. Generally, the arrays comprise 96, 384 or 1536 liquid dispensing tips. Non-limiting examples of automated liquid handling systems include digital dispensers (e.g., HP D300 Digital Dispenser) and pinning machines (e.g., MULTI-BLOT™ Replicator System, CyBio, Perkin Elmer Janus). Non-automated methods are also contemplated by the disclosure, and include but are not limited to a manual digital repeat multichannel pipette.

The amount of adenosine diphosphate (ADP) produced in the presence and absence of the candidate compound can be quantified by any suitable method known in the art. For example, the production of ADP can be quantified by colorimetric assay, fluorometric assay, spectroscopic assay (e.g., stable isotope dilution mass spectrometry), or biochemical assay. In some embodiments, the amount of ADP produced is quantified using luminescence or radioactivity. In some embodiments, the amount of ADP is quantified using the ADP-Glo™ Kinase assay.

The amount of time that the Polθ or fragment thereof, ATP and ssDNA substrate are incubated in the presence or absence of the candidate compound can vary. In some embodiments, incubation time ranges from about 1 hour to about 36 hours. In some embodiments, incubation time ranges from about 5 hours to about 20 hours. In some embodiments, incubation time ranges from about 2 hours to about 18 hours. In some embodiments, the Polθ or fragment thereof, ATP and ssDNA substrate are incubated in the presence or absence of the candidate compound for at least 2 hours, 4 hours, 8, hours, 10 hours, 12 hours, 14 hours, 16 hours, or 18 hours.

The amount of Polθ or fragment thereof used in methods described by the disclosure can vary. In some embodiments, the amount of Polθ or fragment thereof ranges from about 1 nM to about 100 nM. In some embodiments, the amount of Polθ or fragment thereof ranges from about 10 nM to about 50 nM. In some embodiments, the amount of Polθ or fragment thereof ranges from about 5 nM to about 20 nM. In some embodiments, 5 nM, 10 nm or 15 nm of Polθ or a fragment thereof is used.

The amount of ATP used in methods described by the disclosure can vary. In some embodiments, the amount of ATP ranges from about 1 nM to about 200 nM. In some embodiments, the amount of ATP ranges from about 10 nM to about 175 nM. In some embodiments, the amount of ATP ranges from about 5 nM to about 150 nM. In some embodiments, 25, 50, 100, 125, 150, or 175 μM of ATP is used.

A candidate compound can be identified as an inhibitor of the ATPase activity of Polθ if the amount of ADP produced in the presence of the candidate compound is less than the amount produced in the absence of candidate compound. The amount of ADP produced in the presence of an inhibitor of the ATPase activity of Polθ can range from about 2-fold less to about 500-fold less, 5-fold less to about 250-fold less, 10-fold less to about 150-fold less, or about 20-fold less to about 100-fold less, than the amount of ADP produced in the absence of the inhibitor of the ATPase activity of Polθ. In some embodiments, the amount of ADP produced in the presence of an inhibitor of the ATPase activity of Polθ can range from about 100% to about 1% less, about 90% to about 10% less, about 80% to about 20% less, about 70% to about 30% less, about 60% to about 40% less than the amount of ADP produced in the absence of the inhibitor of the ATPase activity of Po10.

In some embodiments, high-throughput screening is carried out in a multi-well cell culture plate. In some embodiments, the multi-well plate is plastic or glass. In some embodiments, the multi-well plate comprises an array of 6, 24, 96, 384 or 1536 wells. However, the skilled artisan recognizes that multi-well plates may be constructed into a variety of other acceptable configurations, such as a multi-well plate having a number of wells that is a multiple of 6, 24, 96, 384 or 1536. For example, in some embodiments, the multi-well plate comprises an array of 3072 wells (which is a multiple of 1536).

The present invention is further illustrated by the following Example, which in no way should be construed as further limiting. The entire contents of all of the references (including literature references, issued patents, published patent applications, and co-pending patent applications) cited throughout this application are hereby expressly incorporated by reference.

### EXAMPLES

### Example 1: Polθ Expression and Homologous Recombination in Cancer

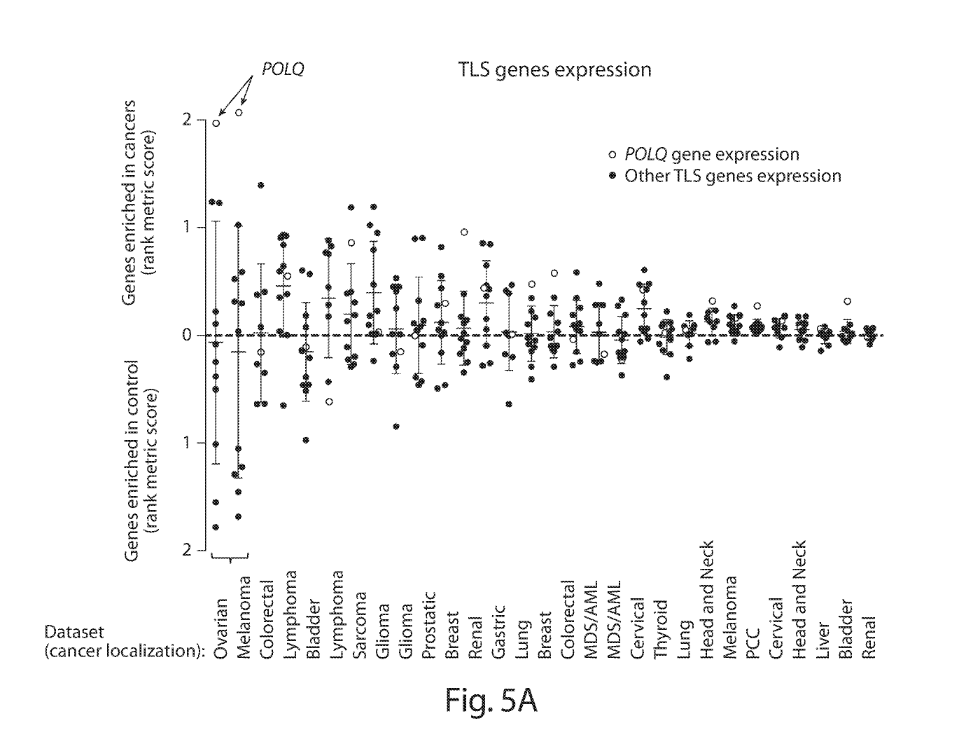

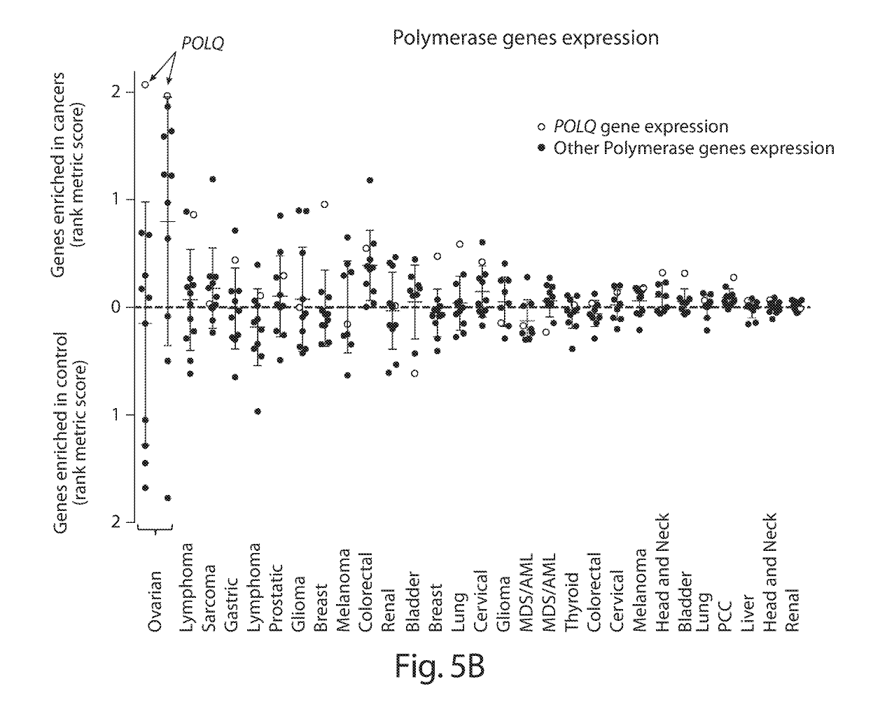

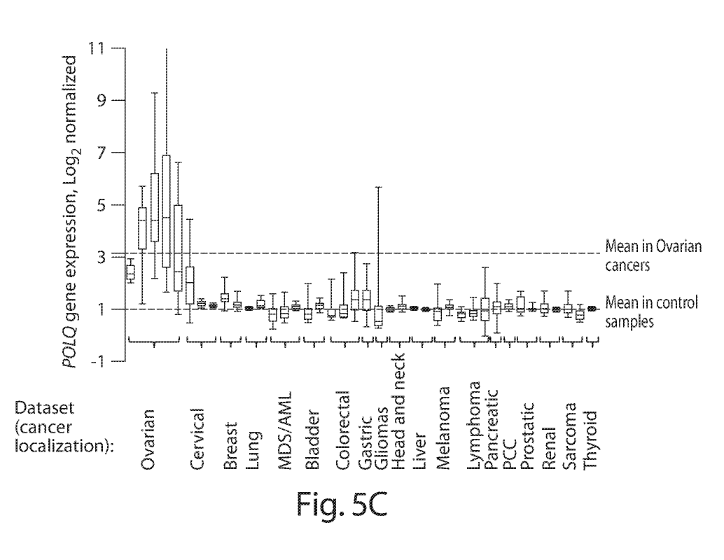

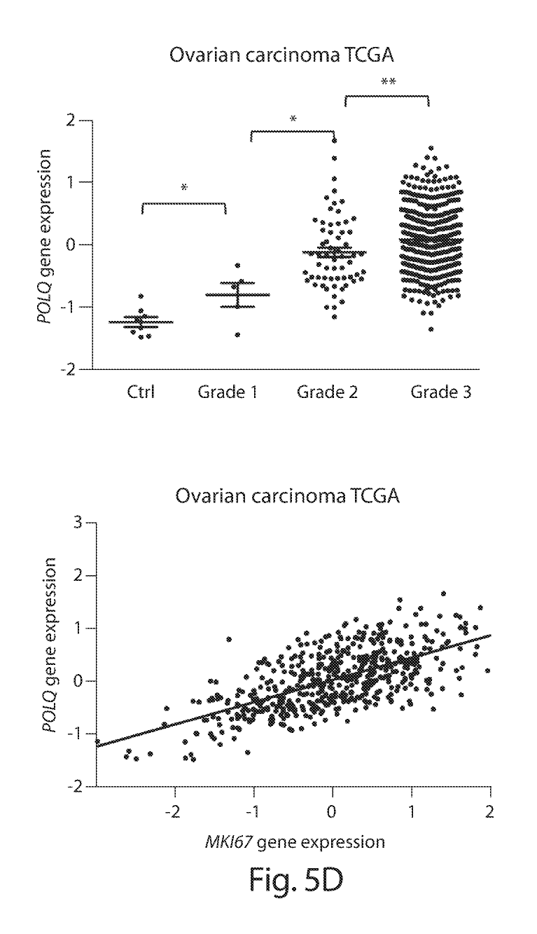

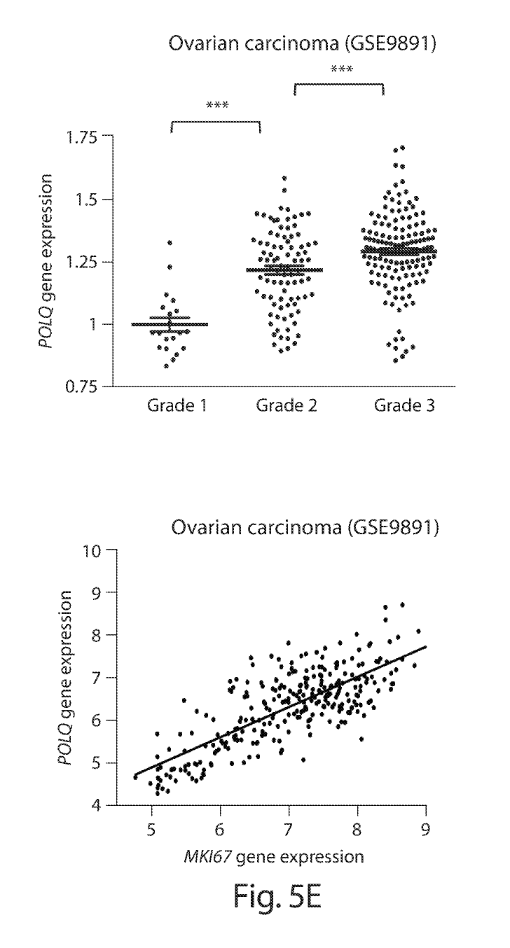

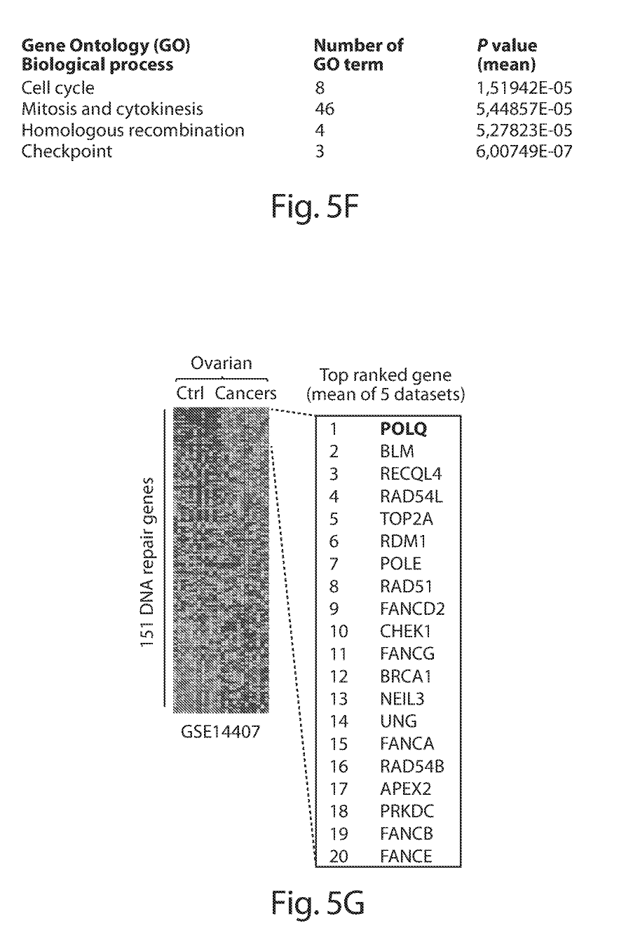

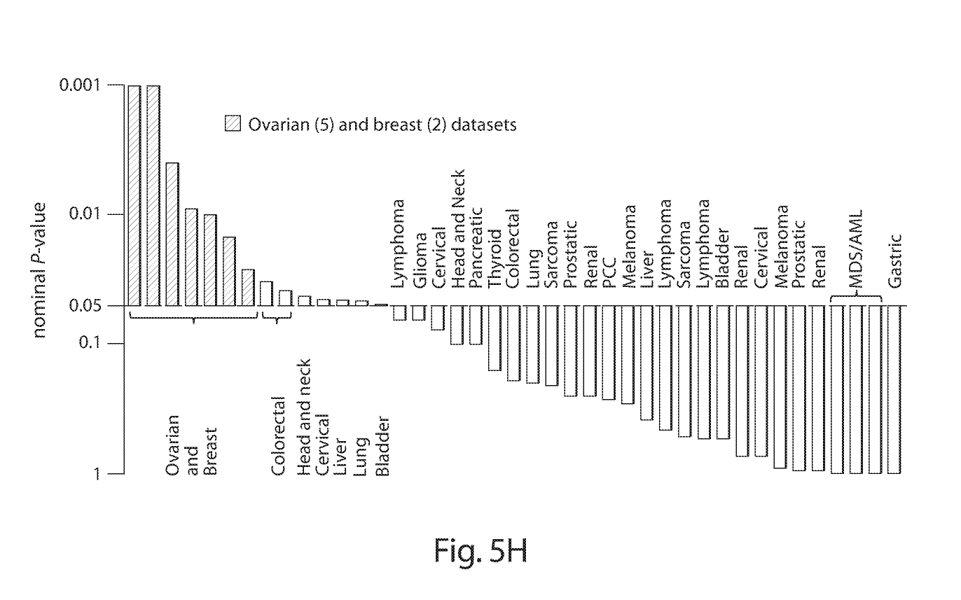

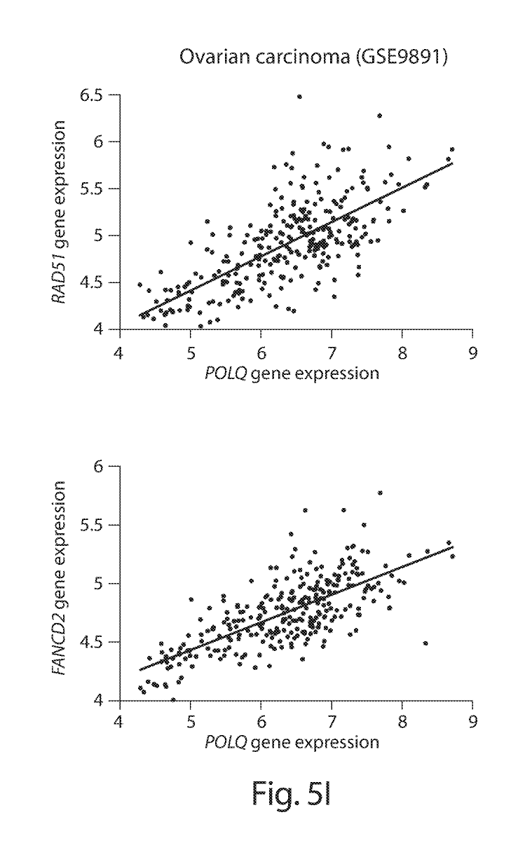

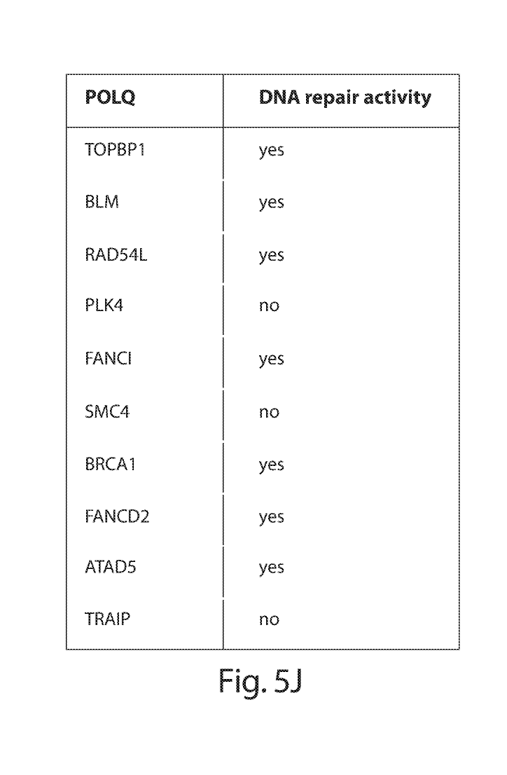

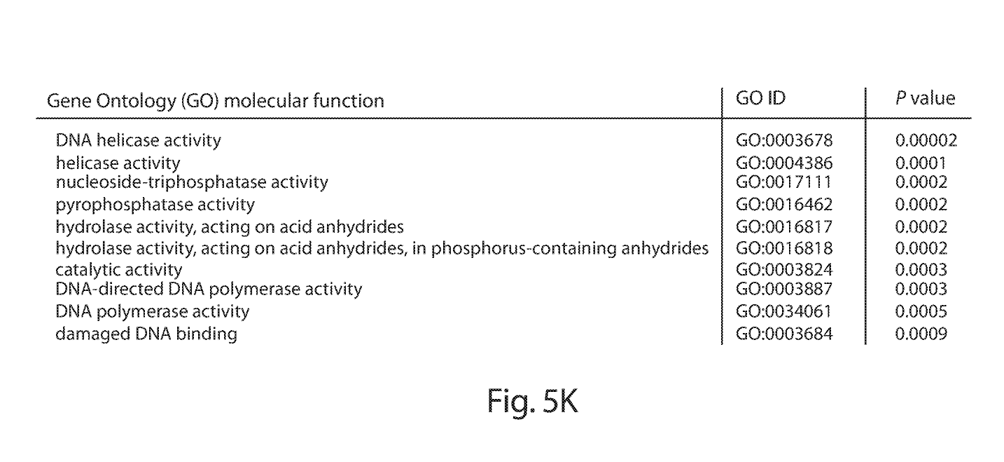

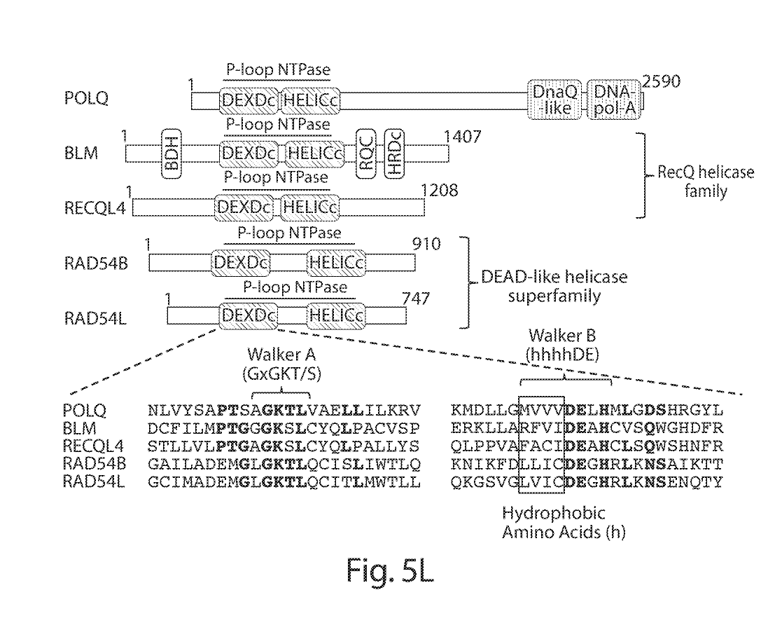

To examine changes in polymerase activity between tumors and normal tissues, polymerase gene expression profiles were screened in a broad number of cancers. Gene set enrichment analysis (GSEA) revealed specific and recurrent overexpression of POLQ in EOCs (FIGS. 5A-5C). POLQ was up-regulated in a grade-dependent manner and its expression positively correlated with numerous mediators of HR (FIGS. 5D-5J). Since POLQ has been suggested to play a role in DNA repair^{-/- 10}, a potential role for POLQ in HR repair was investigated.

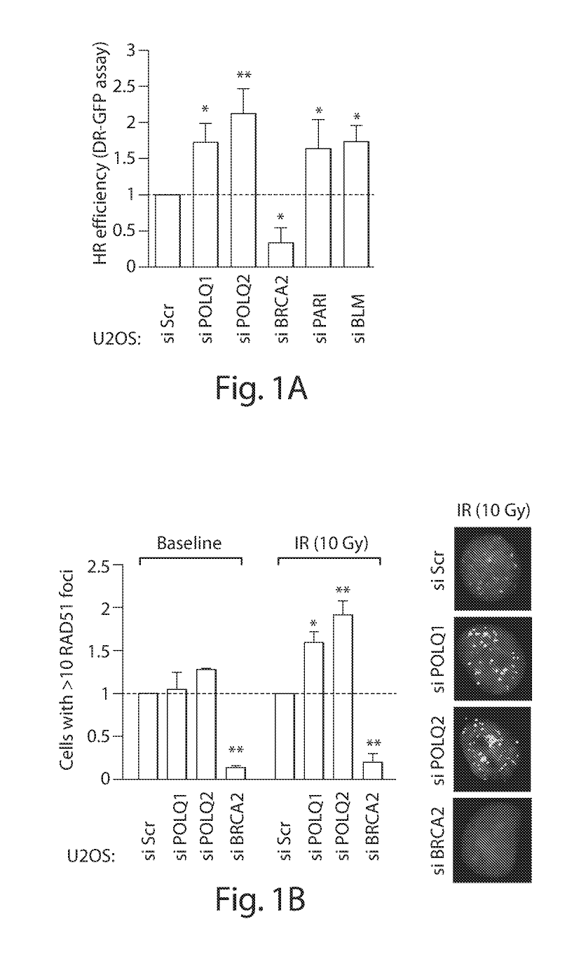

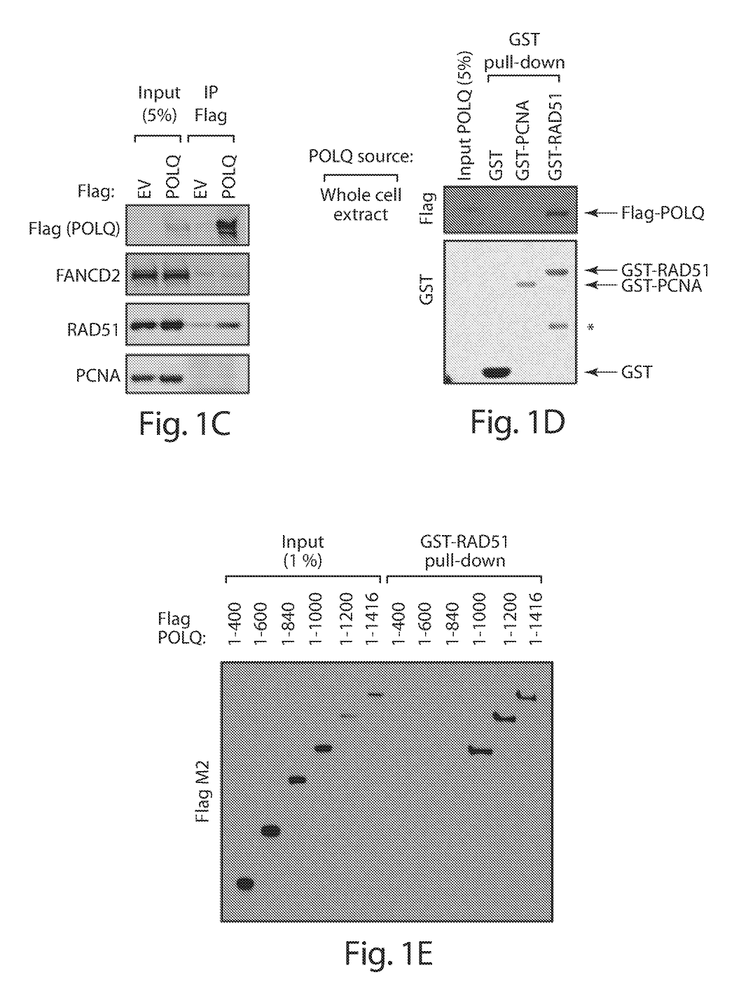

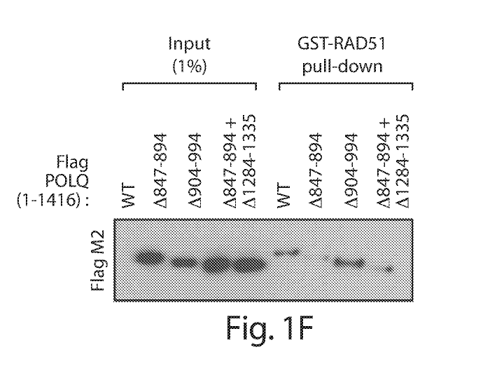

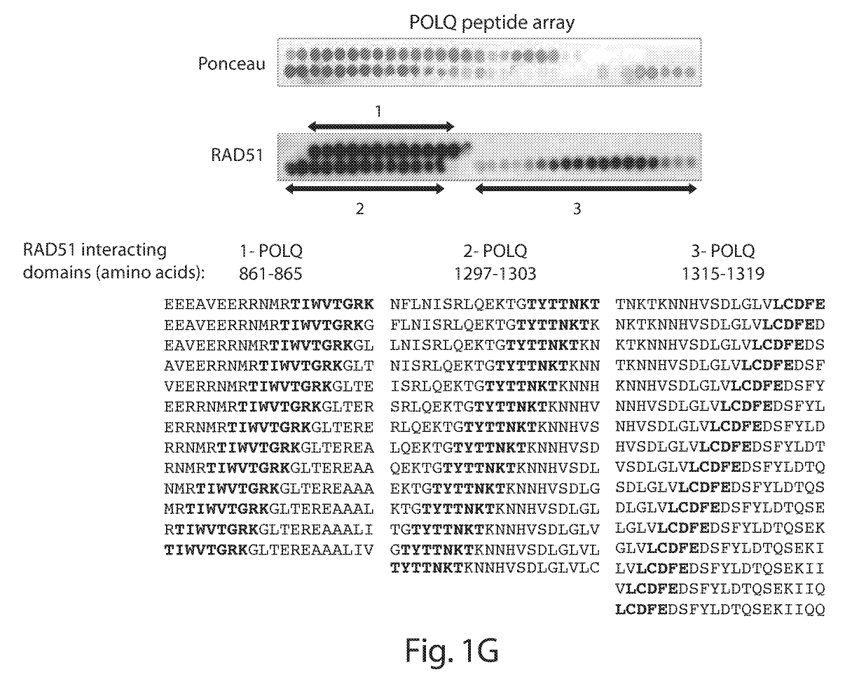

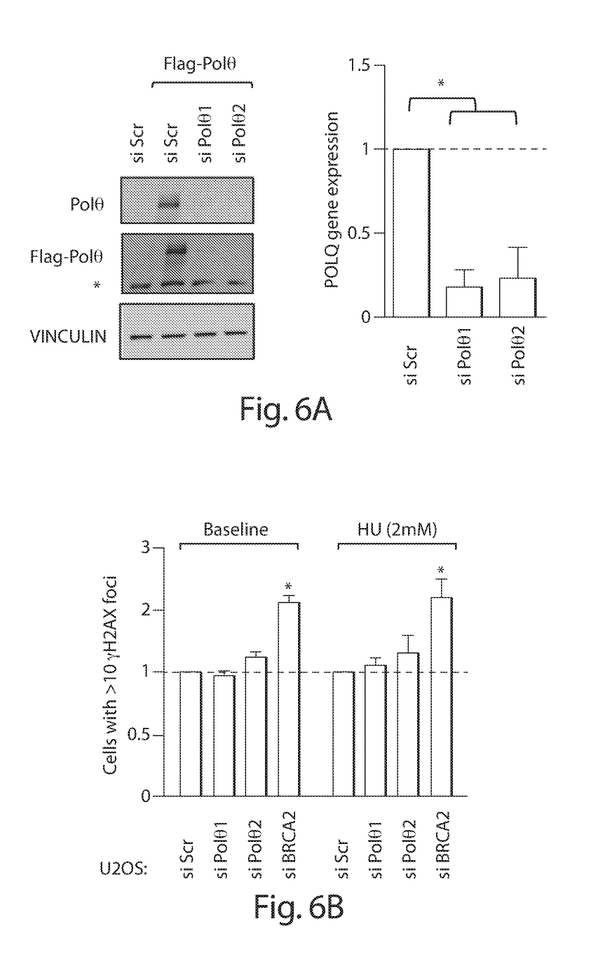

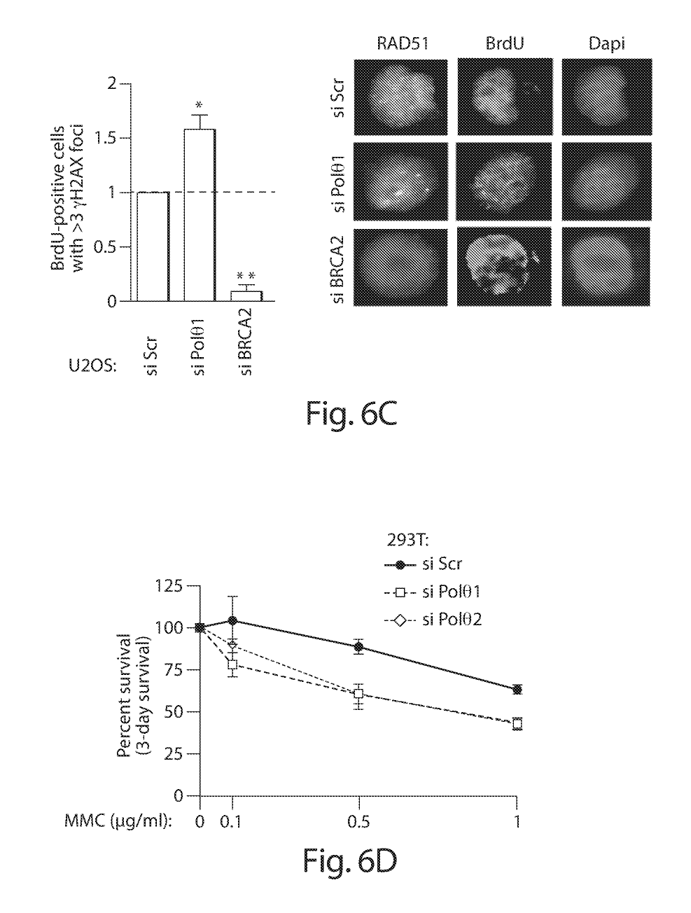

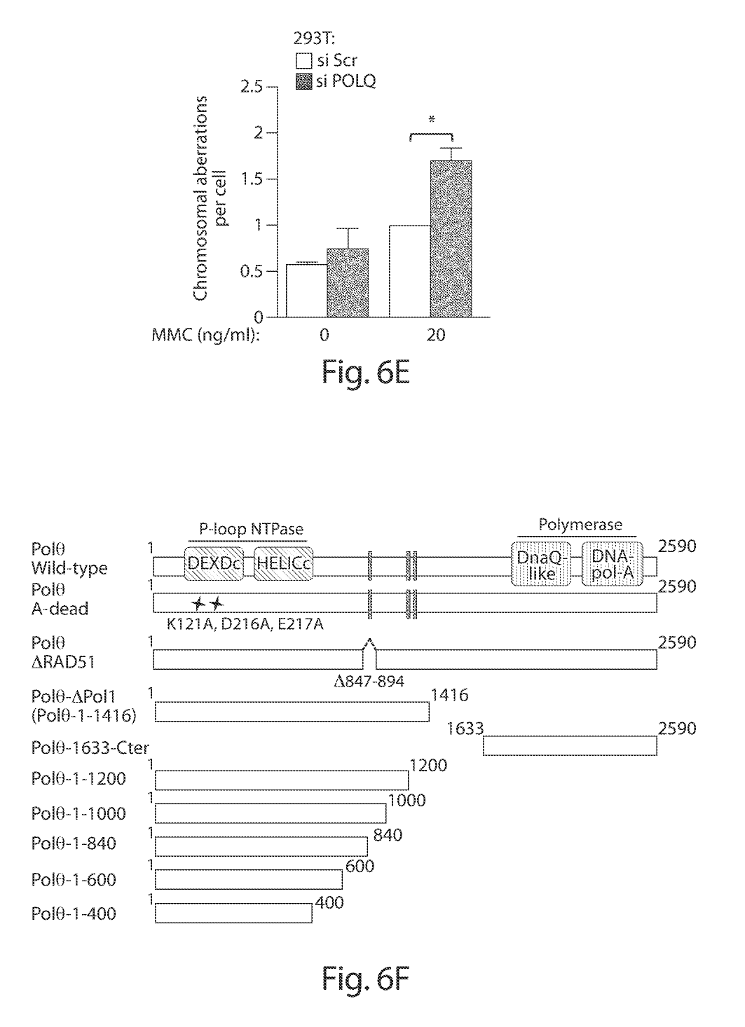

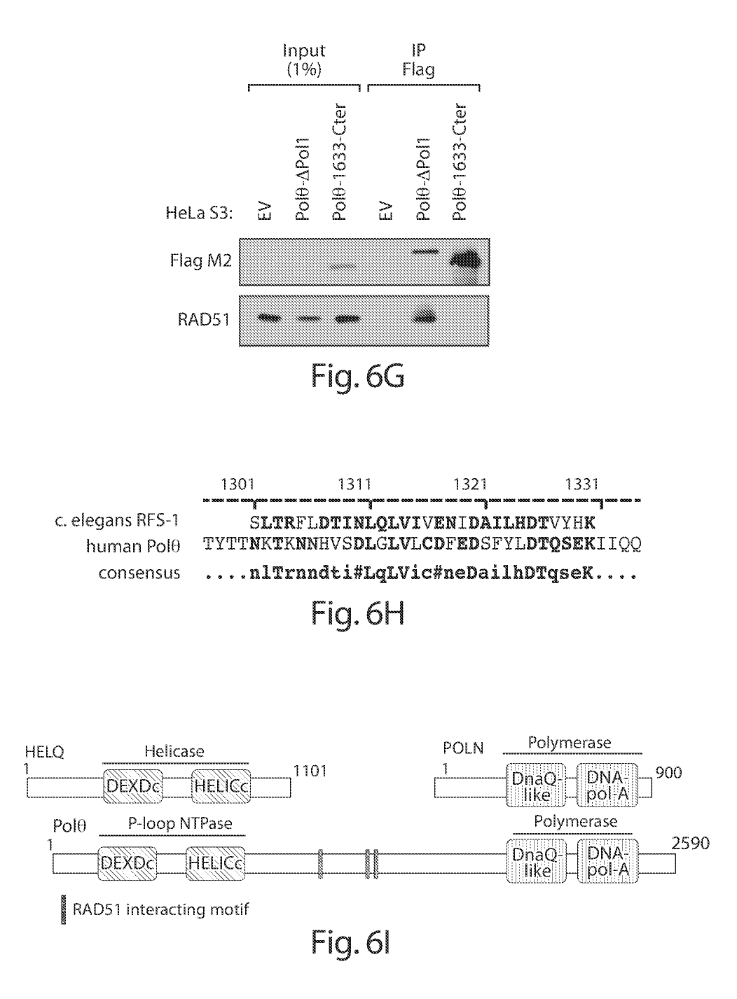

To test the relationship between POLQ expression and HR, a cell-based assay was used which measures the efficiency of recombination of two GFP alleles (DR-GFP)¹⁴. Knockdown of POLQ with siRNA (FIG. 6A) resulted in an increase in HR efficiency, similar to that observed by depleting the anti-recombinases PARI or BLM^{15,16}. Depletion of POLQ caused a significant increase in basal and radiation (IR)-induced RAD51 foci (FIGS. 1A-1B and FIGS. 6B-6C), and depletion of POLQ in 293T cells conferred cellular hypersensitivity to mitomycin C (MMC) and an increase in MMC-induced chromosomal aberrations (FIGS. 6D-6E). These findings suggest that human POLQ inhibits HR and participates in the maintenance of genome stability.

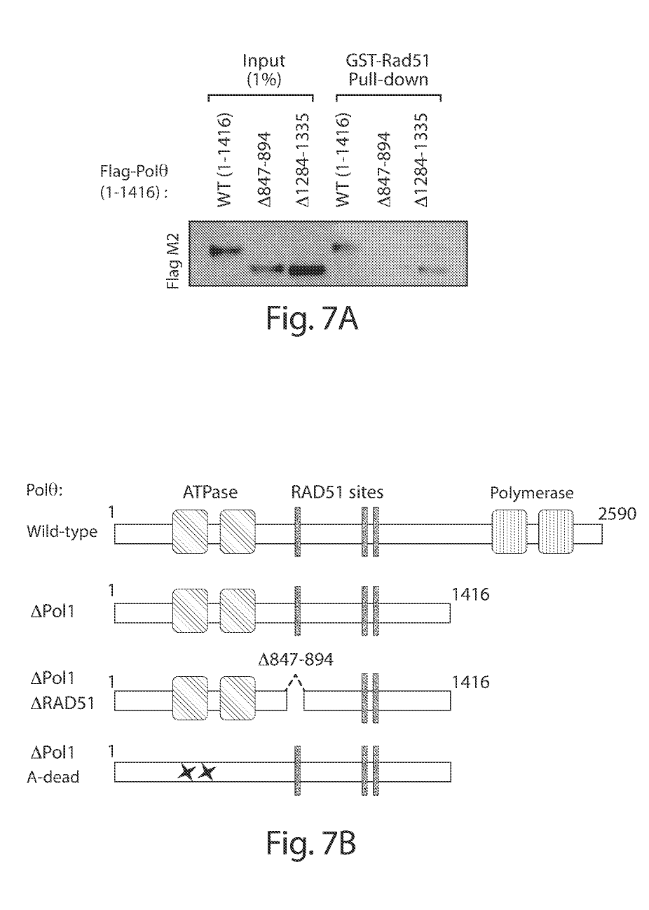

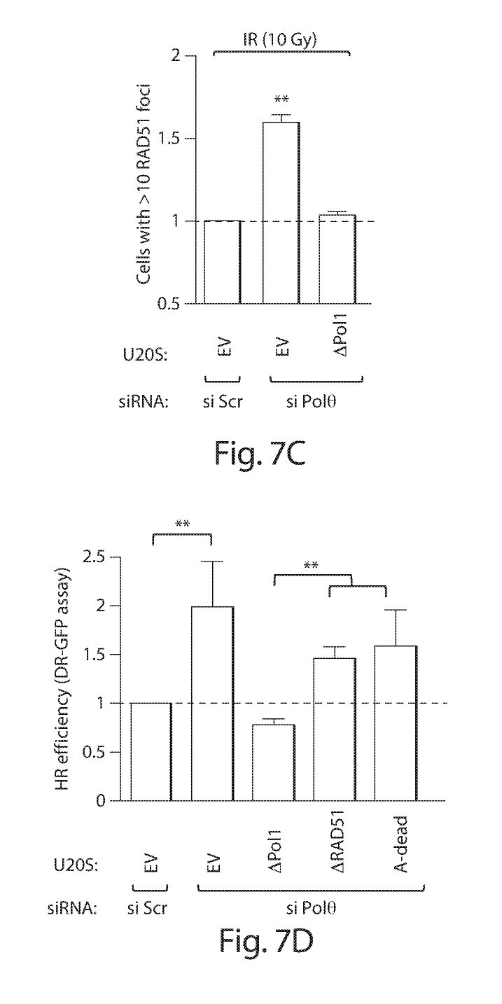

Given that POLQ shares structural homology with coexpressed RAD51-binding ATPases (FIGS. 5K-5L), it was hypothesized that POLQ might regulate HR through an interaction with RAD51. Indeed, RAD51 was detected in Flag-tagged POLQ immunoprecipitates, and purified full-length Flag-POLQ bound recombinant human RAD51 (FIGS. 1C-1D). Pull-down assays with recombinant GST-RAD51 and in vitro translated POLQ truncation mutants defined a region of POLQ binding to RAD51 spanning amino acid 847-894 (FIGS. 1E-1F and FIGS. 6F-6G). Sequence homology of POLQ with the RAD51 binding domain of C. elegans RFS-1¹⁷ identified a second binding region (FIG. 6H). Peptides arrays narrowed down the RAD51 binding activity of POLQ to three distinct motifs (FIG. 1G and FIG. 6I). Substitution arrays confirmed the interaction and highlighted the importance of the 847-894 POLQ region as both necessary and sufficient for RAD51 binding (FIG. 7A). Taken together, these results indicate that POLQ is a RAD51-interacting protein that regulates HR.

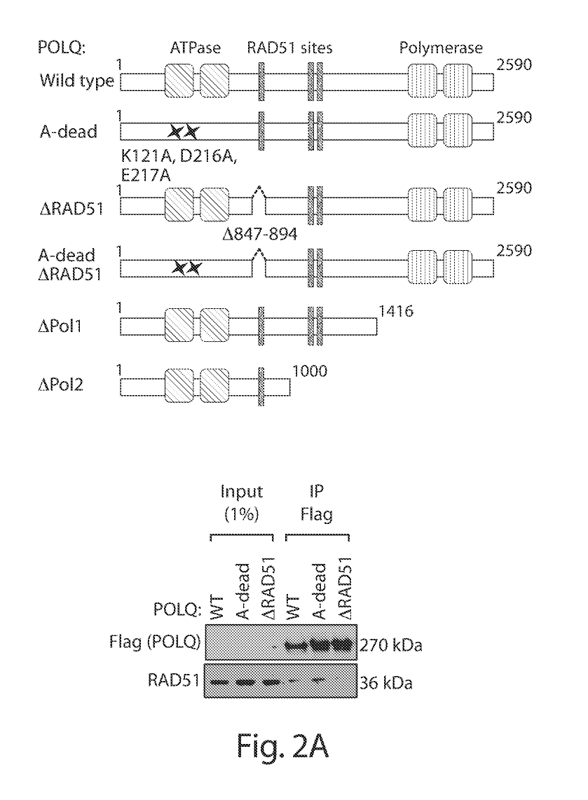

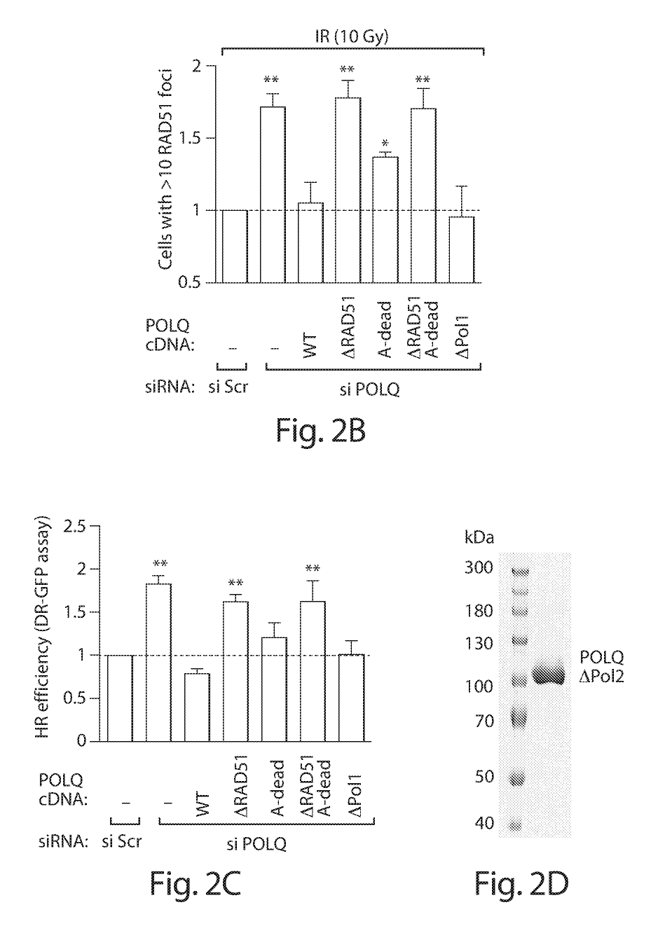

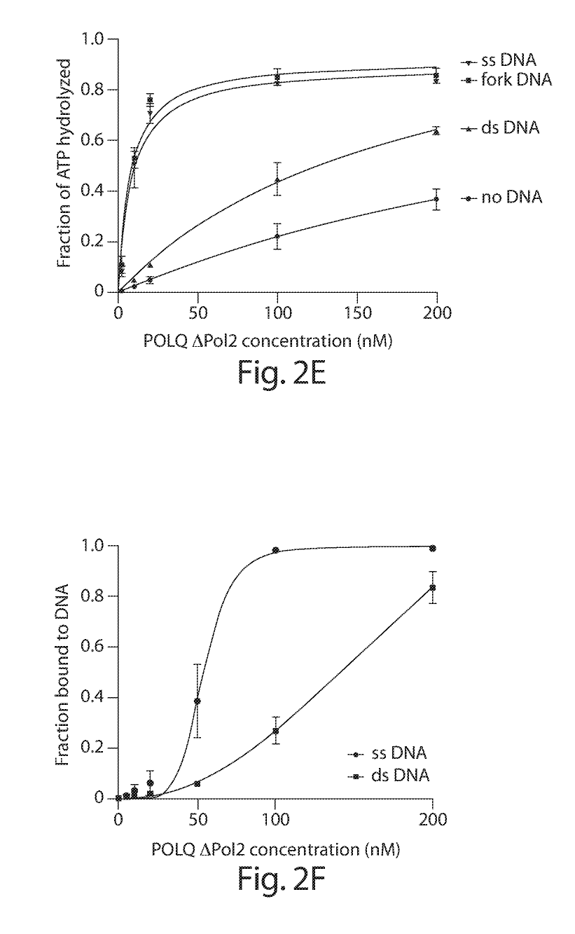

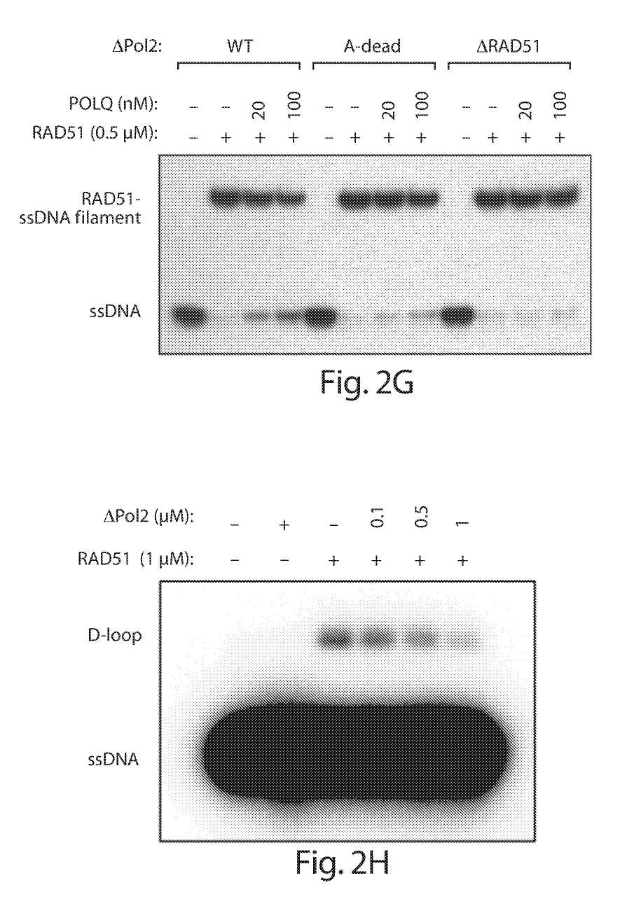

In order to address the role of POLQ in HR regulation, the ability of wild-type (WT) or mutant POLQ to complement the siPOLQ-dependent increase in RAD51 foci was assessed. Full-length wild-type POLQ fully reduced IR-induced RAD51 foci, unlike POLQ mutated at ATPase catalytic residues (A-dead) or POLQ lacking interaction with RAD51 (ΔRAD51) (FIGS. 2A-2B). Expression of a POLQ mutant lacking the polymerase domain (ΔPol1) was sufficient to decrease IR-induced RAD51 foci, suggesting that the N-terminal half of POLQ is sufficient to disrupt RAD51 foci (FIG. 2B and FIGS. 7B-7C). The ability of wild-type or mutant POLQ to complement the siPOLQ-dependent increase in HR efficiency was measured. Again, expression of full-length POLQ or ΔPol1 decreased the recombination frequency when compared to cells expressing other POLQ constructs, suggesting that the N-terminal half of POLQ containing the RAD51 binding domain and the ATPase domain is needed to inhibit HR (FIG. 2C and FIG. 7D).

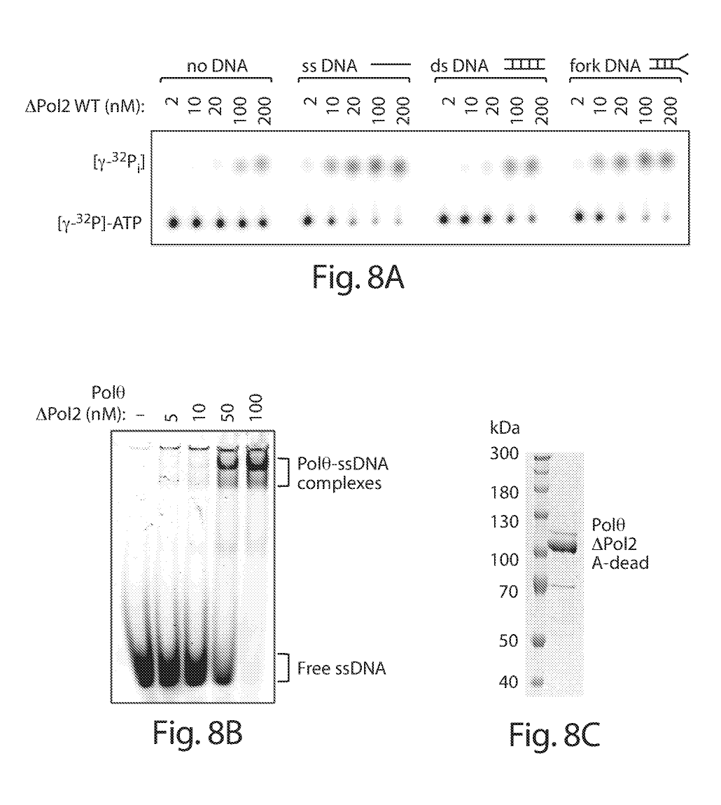

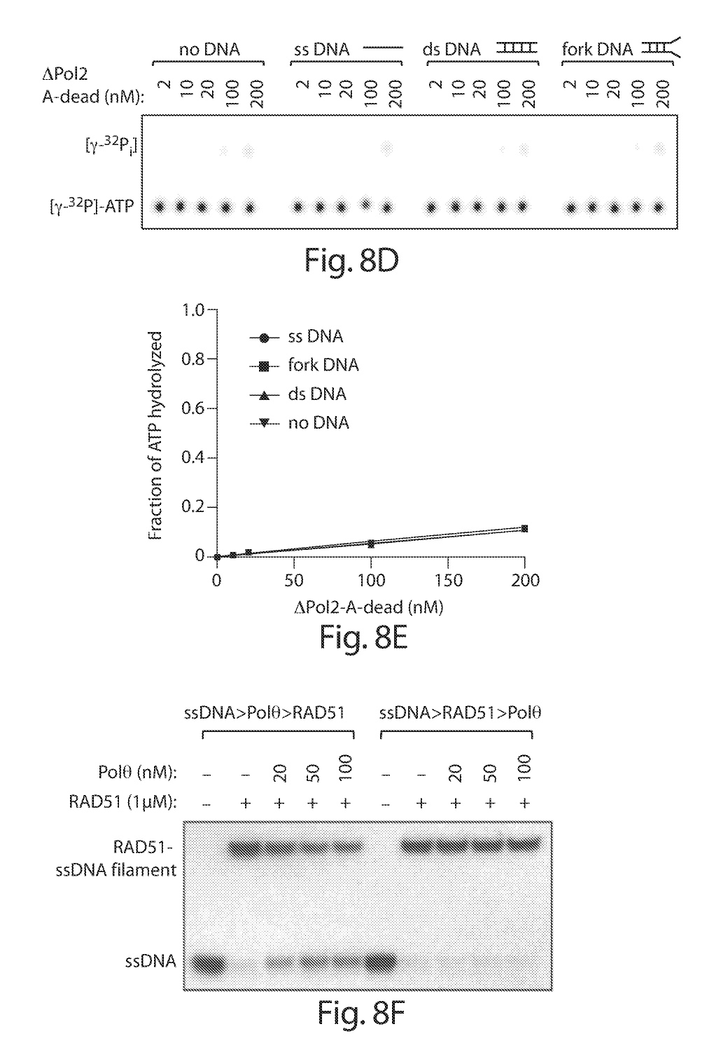

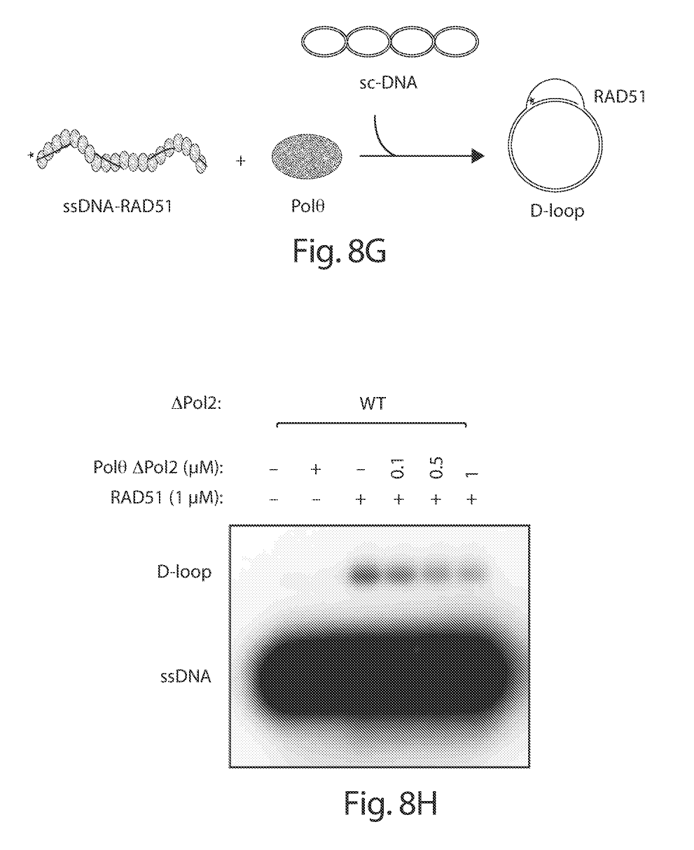

A purified recombinant POLQ fragment (ΔPol2) from insect cells exhibited low levels of basal ATPase activity, as previously reported¹⁸ (FIGS. 2D-2E). POLQ ATPase activity was selectively stimulated by the addition of single-stranded DNA (ssDNA) or fork DNA (FIGS. 2E and 8A). Electrophoretic mobility gel shift assays (EMSA) showed specific binding of POLQ to ssDNA (FIG. 2F and FIG. 8B). ΔPol2 was incubated with ssDNA and measured RAD51-ssDNA nucleofilament assembly. Interestingly, RAD51-ssDNA assembly was reduced by wild-type ΔPol2 but not by A-dead or ΔRAD51, indicating that POLQ negatively affects RAD51-ssDNA assembly through its RAD51 binding and ATPase activities (FIG. 2G and FIGS. 8C-8F). Furthermore, POLQ decreased the efficiency of D-loop formation, confirming that POLQ is a negative regulator of HR (FIG. 2H and FIGS. 8G-8I and Table 1, below).

TABLE 1
Effect of POLQ expression levels and HR status on tumor sensitivity to
cisplatin or PARPi.

| | RAD51 | | RAD51-ssDNA | D-loop |
|---|---|---|---|---|
| Conditions tested | foci | DR-GFP | assembly | formation |
| si Polθ | ↑ | ↑ | NA | NA |
| Polθ cDNA WT | ↓ | ↓ | ↓ | ↓ |
| Polθ-A-dead cDNA | — | — | — | NA |
| Polθ-ΔRAD51 cDNA | — | — | — | NA |

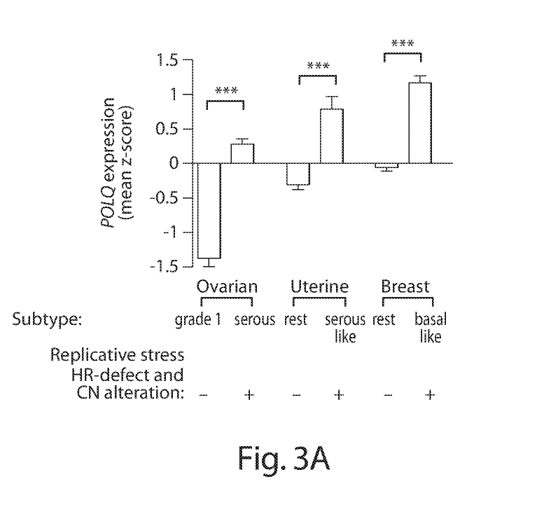

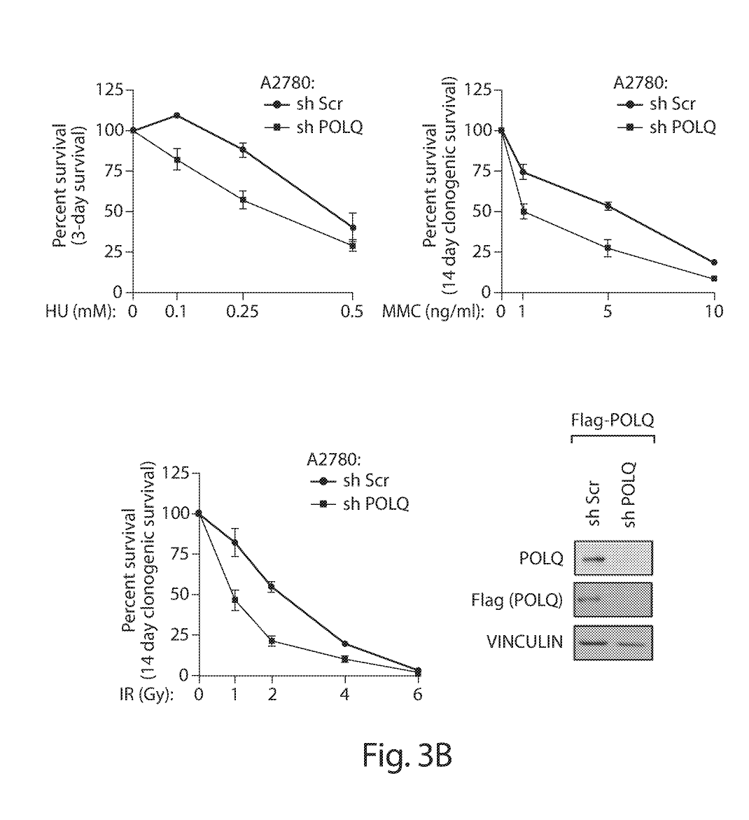

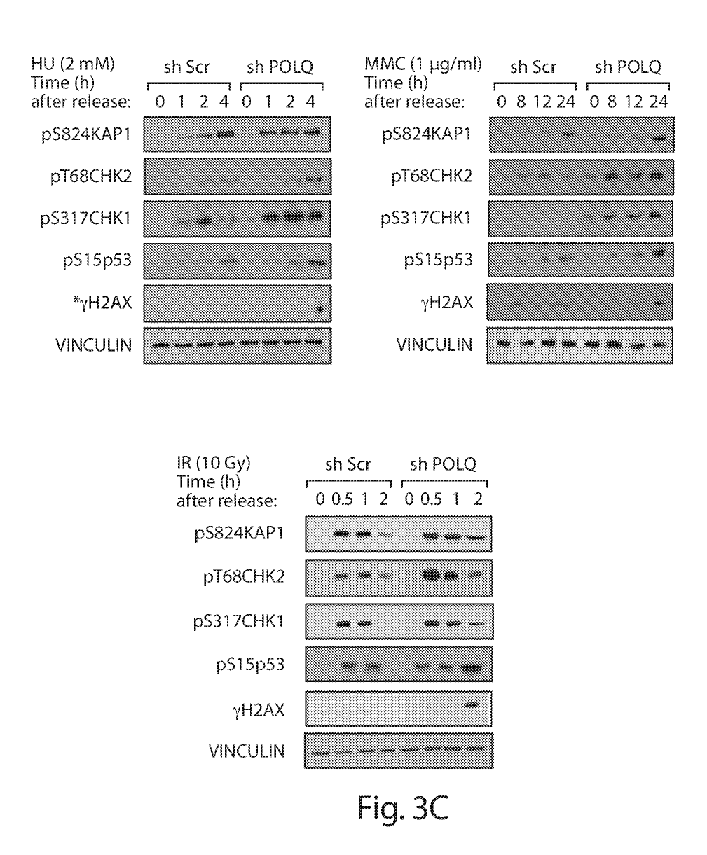

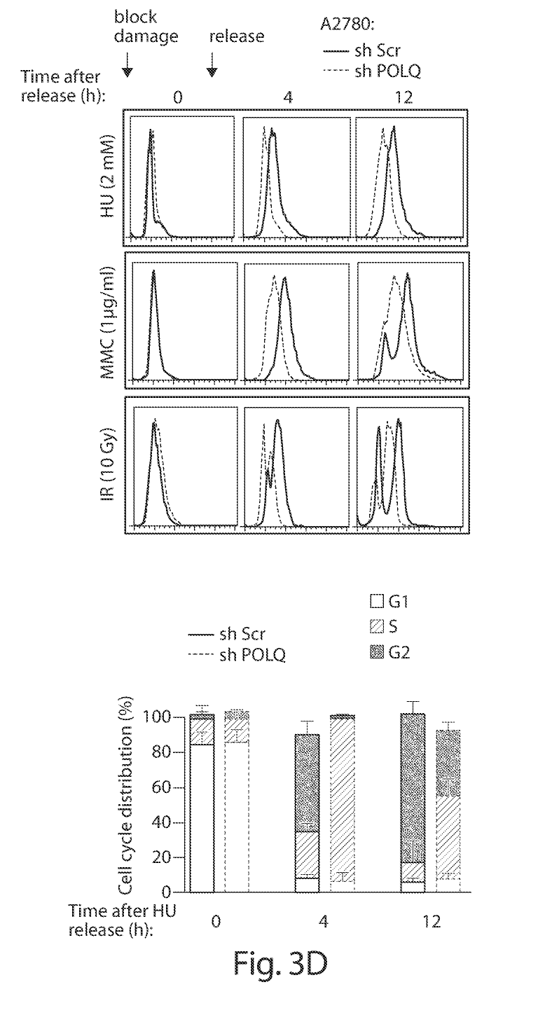

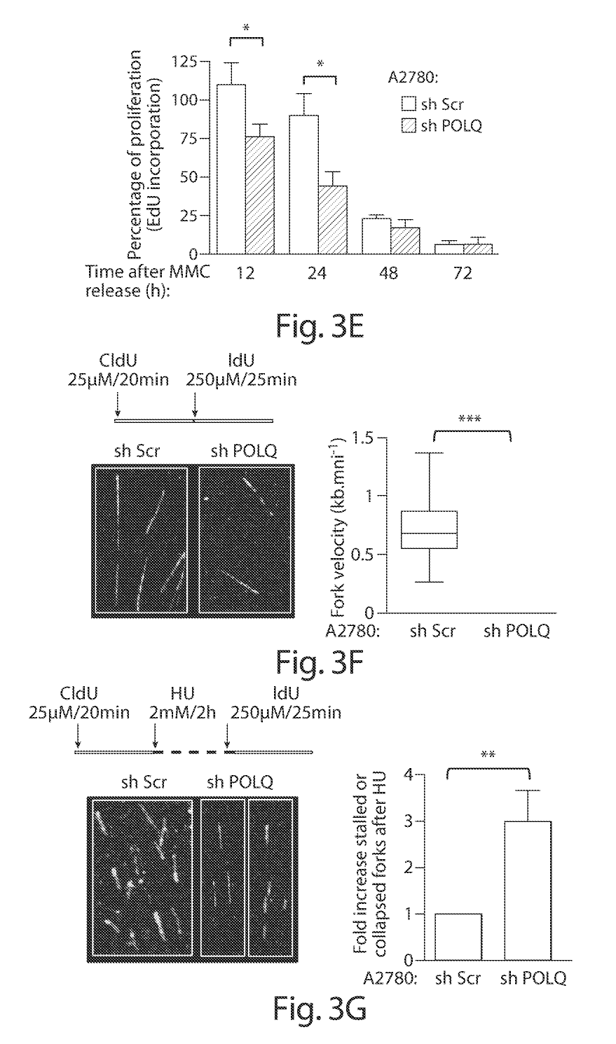

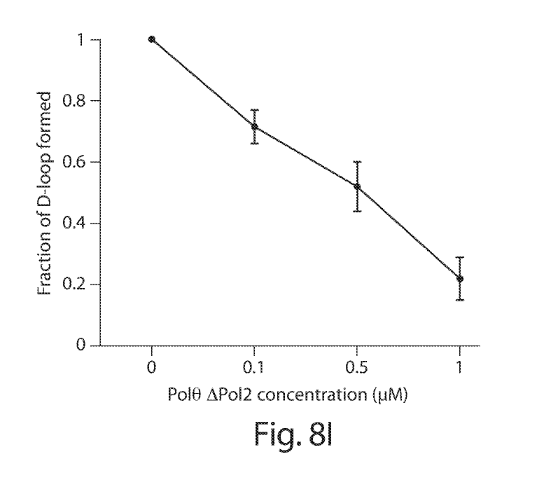

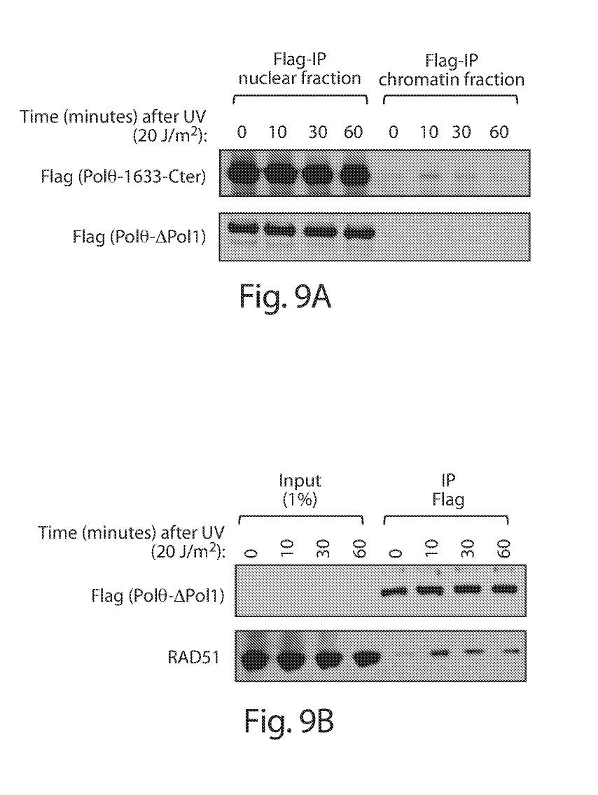

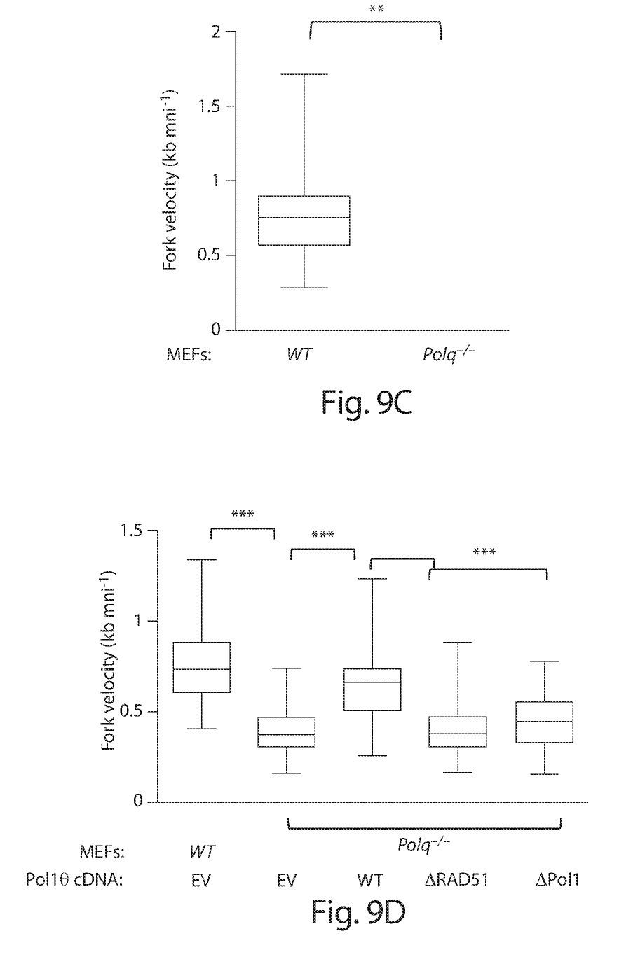

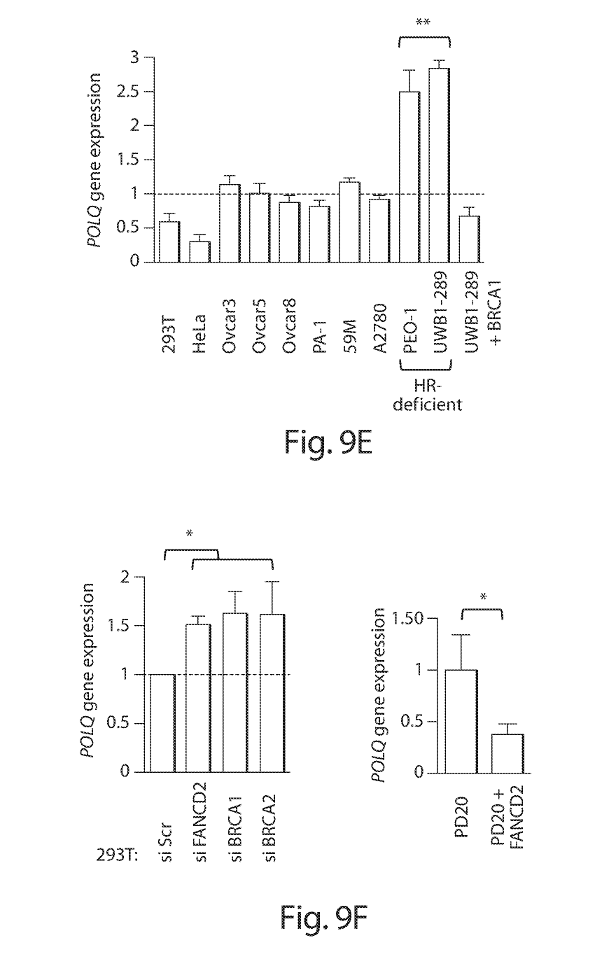

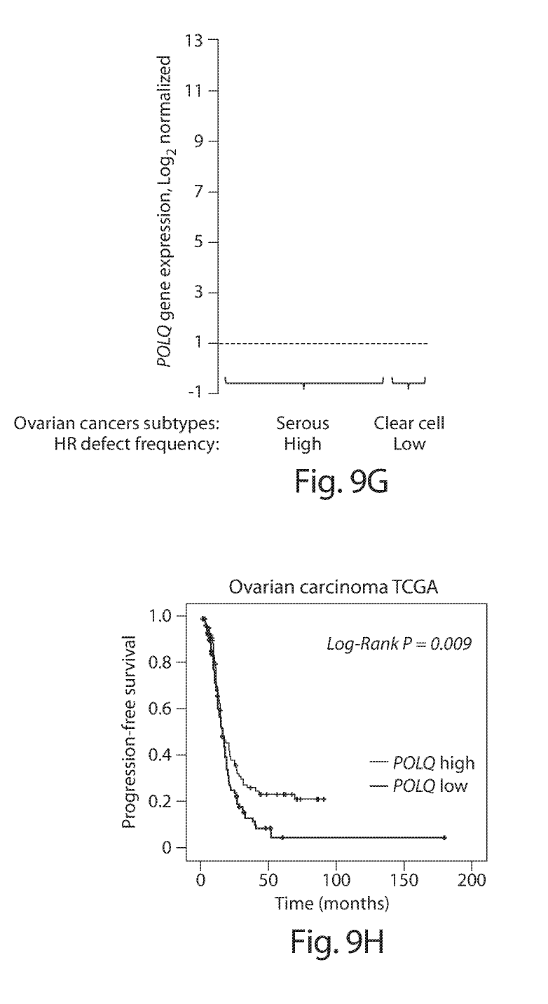

Since POLQ is up-regulated in subgroups of cancers associated with HR deficiency (FIG. 3A) and POLQ activity shows specificity for replicative stress-mediated structures (ss and fork DNA) (FIGS. 2E-2F), the cellular functions of POLQ under replicative stress were examined. Subcellular fractionation revealed that POLQ is enriched in chromatin in response to ultraviolet (UV) light; and RAD51 binding by POLQ was enhanced by UV exposure, suggesting that POLQ regulates HR in cells under replicative stress (FIGS. 9A-9B). POLQ-depleted cells were hypersensitive to cellular stress and DNA damage along with an exacerbated checkpoint activation and increased γH2AX phosphorylation (FIGS. 3B-3C). Furthermore, the cell cycle progression of POLQ-depleted cells was impaired after DNA damage (FIGS. 3D-3E). To determine the role of POLQ in replication dynamics, single-molecule analyses were performed on extended DNA fibers¹⁹. Abnormalities in replication fork progression were observed in POLQ-depleted cells (FIGS. 3F-3G and FIGS. 9C-9D). These results suggest that POLQ maintains genomic stability at stalled or collapsed replication forks by promoting fork restart.

To examine the regulation of POLQ, POLQ expression was quantified by RT-qPCR. POLQ was selectively up-regulated in HR-deficient ovarian cancer cell lines. Complementation of a BRCA1 or FANCD2-deficient cell lines, restored normal HR function and reduced POLQ expression to normal levels. Conversely, siRNA-mediated inhibition of HR genes increased POLQ expression (FIGS. 9E-9F). POLQ expression was significantly higher in subgroups of cancers with HR deficiency and a high genomic instability pattern²⁰ (FIG. 3A and FIG. 9G). Patients with high POLQ expression had a better response to platinum chemotherapy, a surrogate for HR deficiency, suggesting that POLQ expression inversely correlates with HR activity and may be useful as a biomarker for platinum sensitivity (FIGS. 9H-9I). Together, these data indicate that increased POLQ expression is driven by HR deficiency.

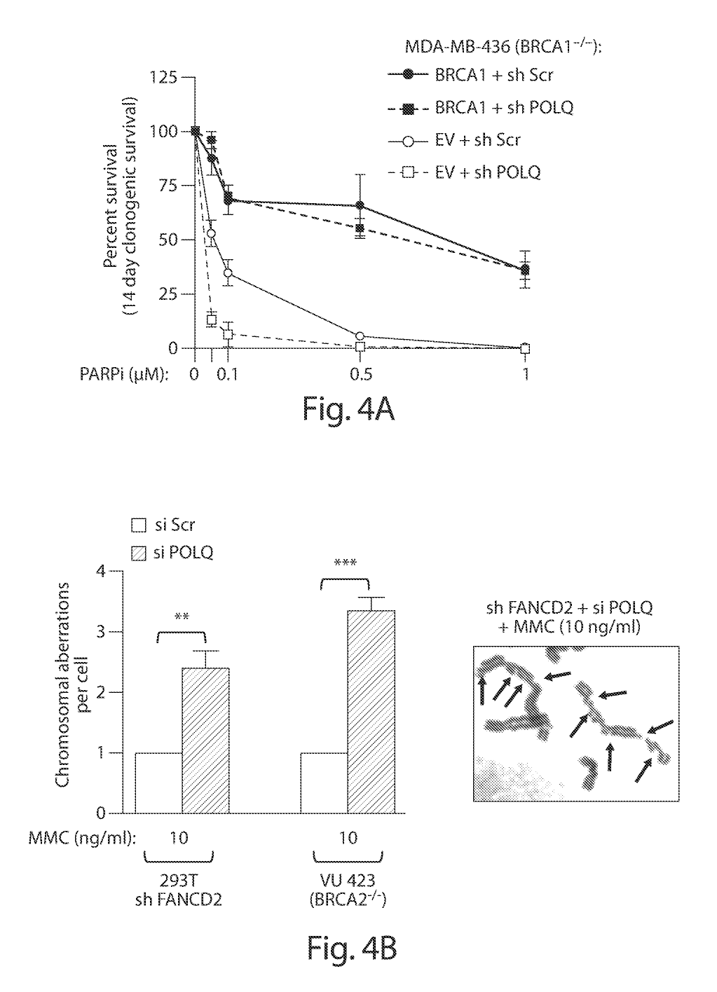

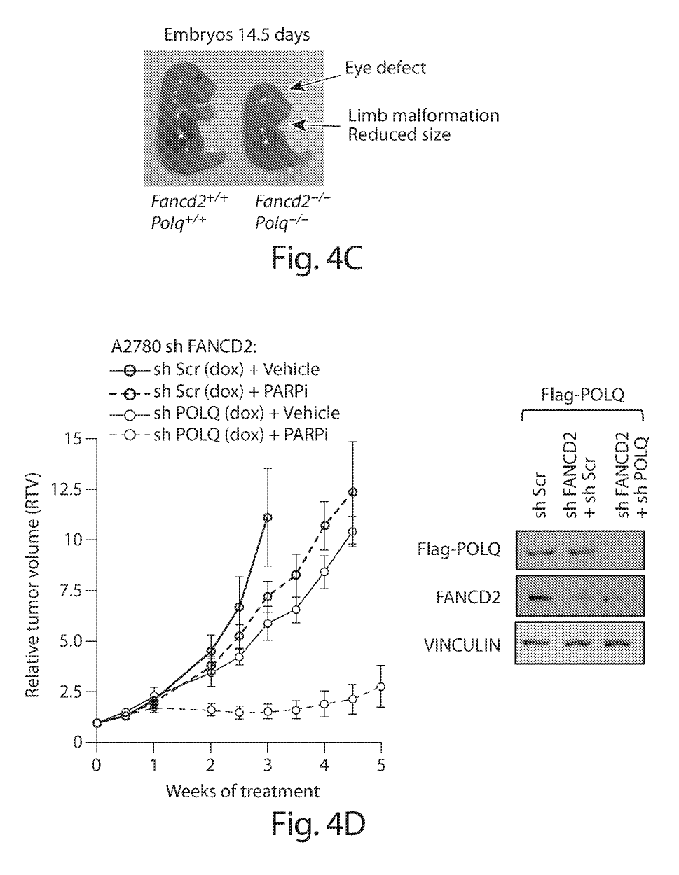

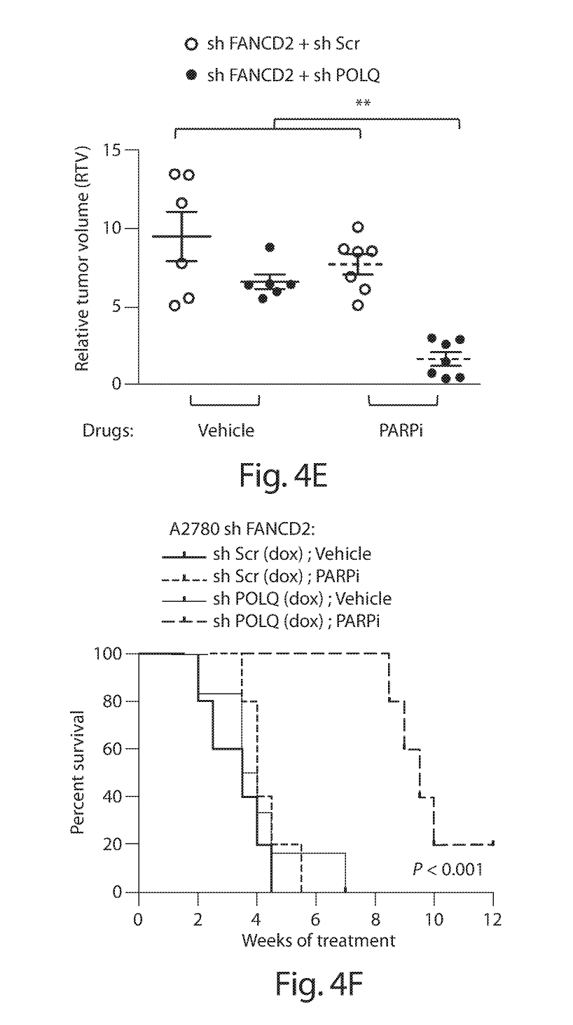

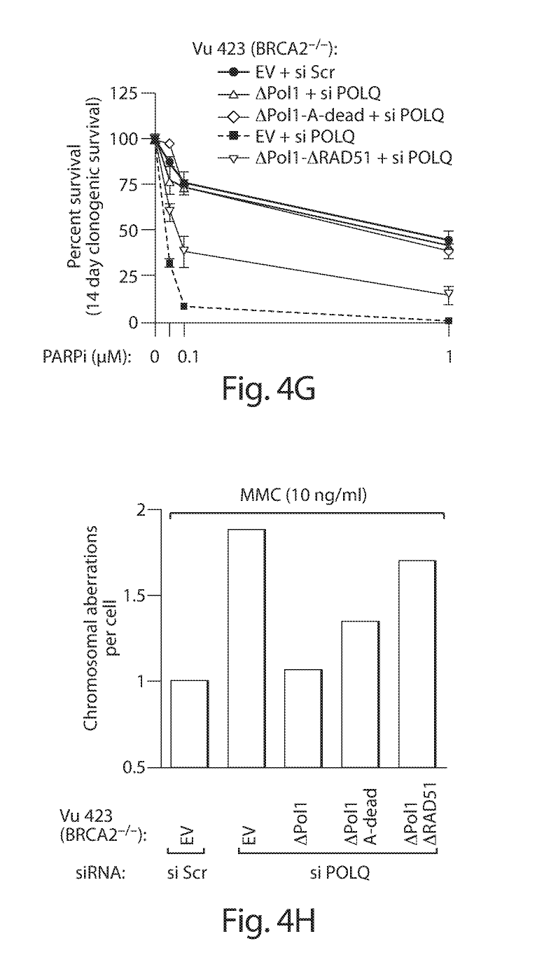

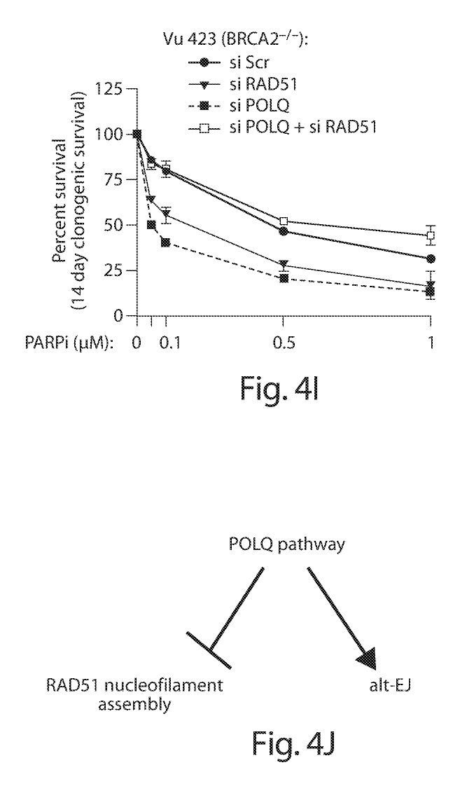

To assess the possible synthetic lethality between HR genes and POLQ, an HR-deficient ovarian tumor cell line, A2780-shFANCD2 cells (FIG. 10A-10C), were generated. These cells, and the parental A2780 cells, were subjected to POLQ depletion, and survival following exposure to cytotoxic drugs was measured. POLQ depletion reduced the survival of HR-deficient cells exposed to inhibitors of PARP (PARPi), cisplatin (CDDP), or MMC (FIGS. 10D-10F). POLQ inhibition impaired the survival of BRCA1-deficient tumors (MDA-MB-436) after PARPi treatment but had no effect on the complemented line (MDA-MB-436+BRCA1) (FIG. 4A). POLQ-depleted cells were hypersensitive to ATM inhibition, known to create an HR defect phenotype²¹. Chromosomal breakage, checkpoint activation, and γH2AX phosphorylation in response to MMC were exacerbated by POLQ depletion (FIG. 4B and FIGS. 10G-10H).

Furthermore, a whole-genome shRNA screen performed on HR-deficient (FANCA^{-/-}) fibroblasts showed that shRNAs targeting POLQ impair cell survival in MMC (FIG. 10I), suggesting that HR-deficient cells cannot survive in the absence of POLQ.

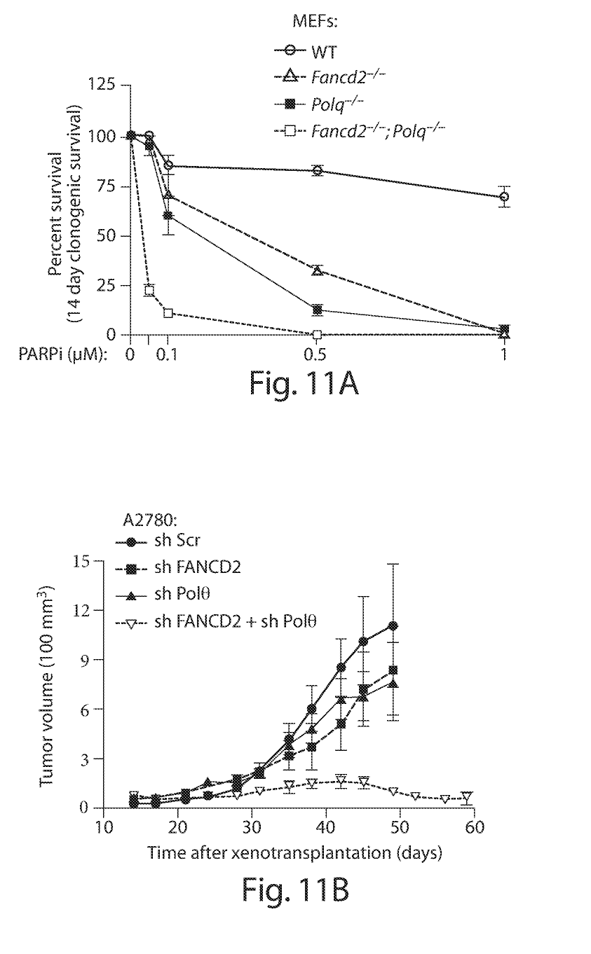

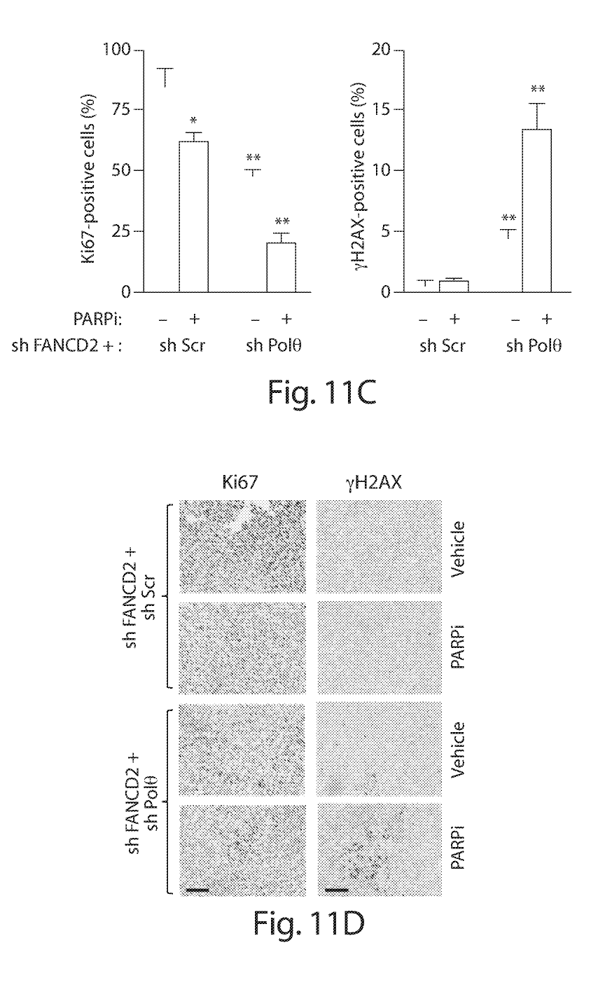

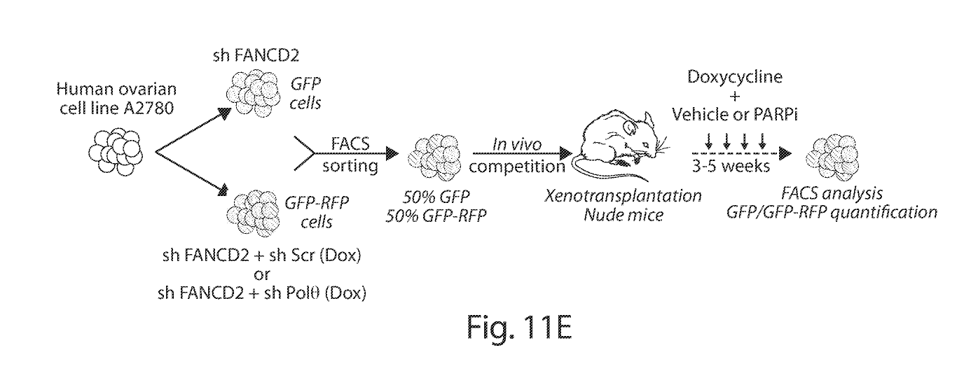

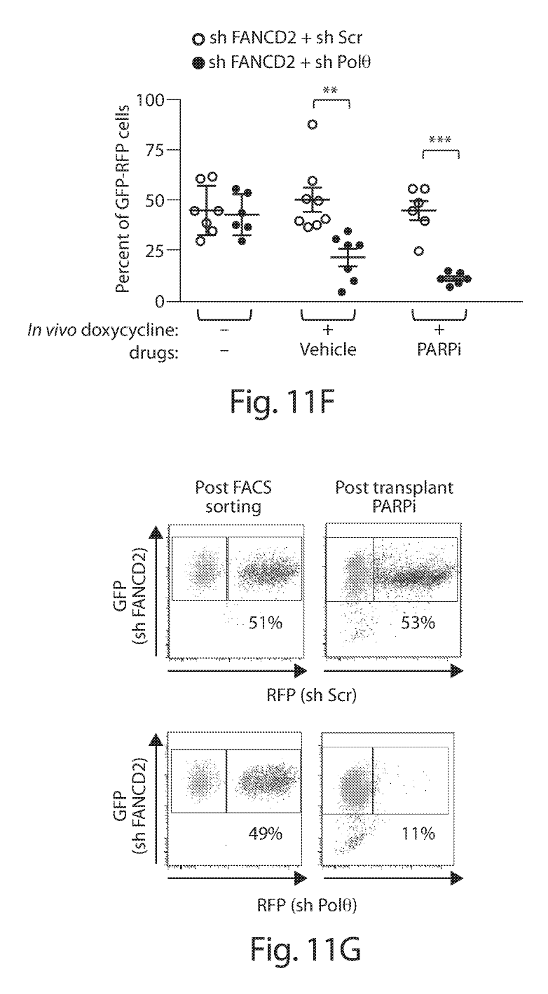

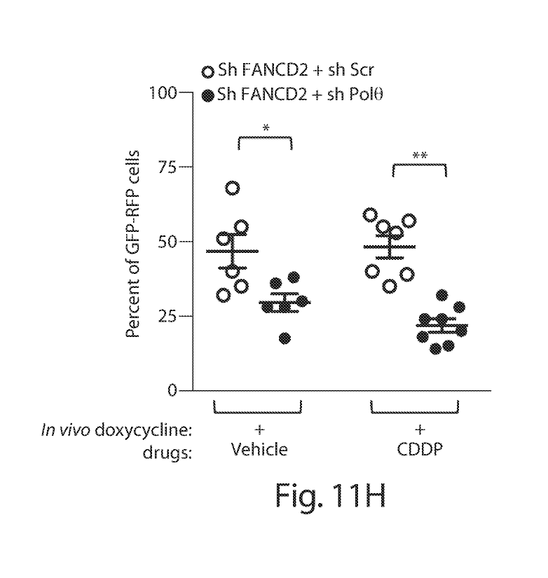

Next, the interaction was investigated between the HR and POLQ pathways in vivo by interbreeding Fancd2^{+/-} and Polq^{-/-} mice. Ψ: four Fancd2^{-/-} Polq^{-/-} offspring were observed with several congenital malformations and premature death within 48 hours of birth. Although Fancd2^{-/-} and Polq^{-/-} mice are viable and exhibit subtle phenotypes^{7,22}, viable Fancd2^{-/-}Polq^{-/-} mice were uncommon from these matings. The only surviving Fancd2^{-/-}Polq^{-/-} pups exhibited severe congenital malformations and were either found dead or died prematurely. Fancd2^{-/-}Polq^{-/-} embryos showed severe congenital malformations, and mouse embryonic fibroblasts (MEFs) generated from Fancd2^{-/-} Polq^{-/-} embryos showed hypersensitivity to PARPi (FIGS. 4C and 11A). These data suggest that loss of the HR and POLQ repair pathways in vivo results in embryonic lethality.

TABLE 2

| Polq | Fancd2 | | % | % | Significant |
|---|---|---|---|---|---|
| status | status | | observed | expected | difference |
| | | Offspring | | | |
| | | observed (n) | | | |
| +/+ | +/+ | 19 | 7.2 | 6.25 | no |
| +/+ | +/− | 43 | 16.2 | 12.5 | no |
| +/+ | −/− | 22 | 8.3 | 6.25 | no |
| +/− | +/+ | 36 | 13.6 | 12.5 | no |
| +/− | +/− | 61 | 23.0 | 25 | no |
| +/− | −/− | 25 | 9.4 | 12.5 | no |
| −/− | +/+ | 21 | 7.9 | 6.25 | no |
| −/− | +/− | 34 | 12.8 | 12.5 | no |
| −/− | −/− | 0^{ψ} | 0 | 6.25 | yes, P < 0.001 |
| Total number: | | 261 | 100 | 100 | |
| | | Embryos | | | |
| | | observed (n) | | | |
| +/+ | +/+ | 13 | 6.6 | 6.25 | no |
| +/+ | +/− | 31 | 15.7 | 12.5 | no |
| +/+ | −/− | 7 | 3.6 | 6.25 | no |
| +/− | +/+ | 22 | 11.2 | 12.5 | no |
| +/− | +/− | 62 | 31.5 | 25 | no |
| +/− | −/− | 28 | 14.2 | 12.5 | no |
| −/− | +/+ | 10 | 5.1 | 6.25 | no |
| −/− | +/− | 16 | 8.1 | 12.5 | no |
| −/− | −/− | 8 | 4.1 | 6.25 | no |

| | | | |
|---|---|---|---|
| Total number: | 197 | 100 | 100 |

| Malformation | % of Polq^{-/-} Fancd2^{-/-} embryos |
|---|---|
| observed | observed with malformations |
| Reduced body weight | 100 |
| Reduced body size | 100 |
| Eye defect | 100 |
| Limb malformation | 12.5 |

Since xenografts of tumors cells expressing shRNAs against both FANCD2 and POLQ did not stably propagate in mice (FIG. 11B), A2780-shFANCD2 cells expressing either doxycycline-inducible POLQ or Scr shRNA were xenotransplanted in athymic nude mice. POLQ depletion significantly impaired tumor growth after PARPi treatment (FIGS. 4D-4E and FIGS. 11C-11D). Moreover, mice bearing POLQ-depleted tumors had a survival advantage following PARPi treatment compared to control mice (FIG. 4F). POLQ-depleted HR-deficient tumor cells also exhibited decreased survival in in vivo dual-color competition experiments (FIG. 11E-11H). Collectively, these data confirm that HR-deficient tumors are hypersensitive to inhibition of POLQ-mediated repair.

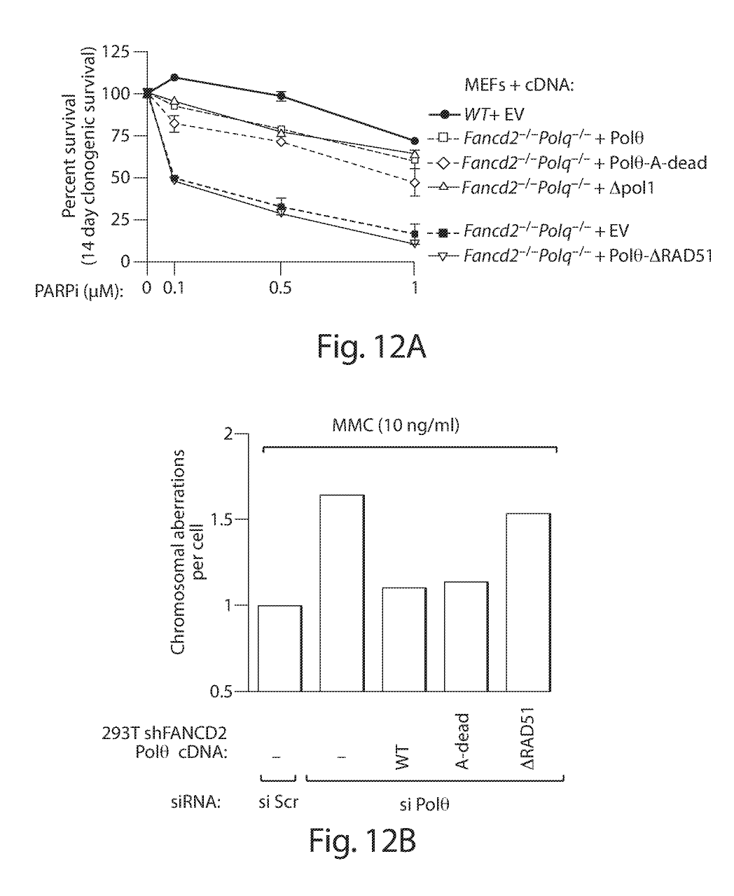

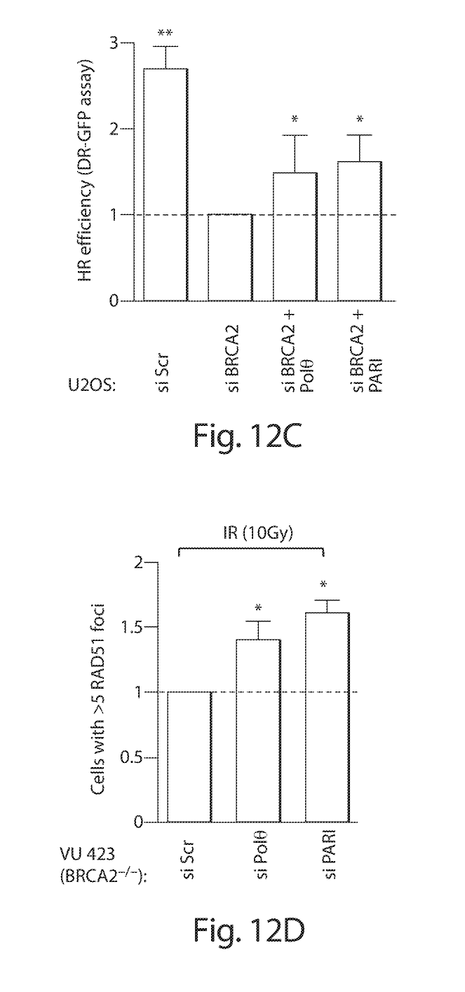

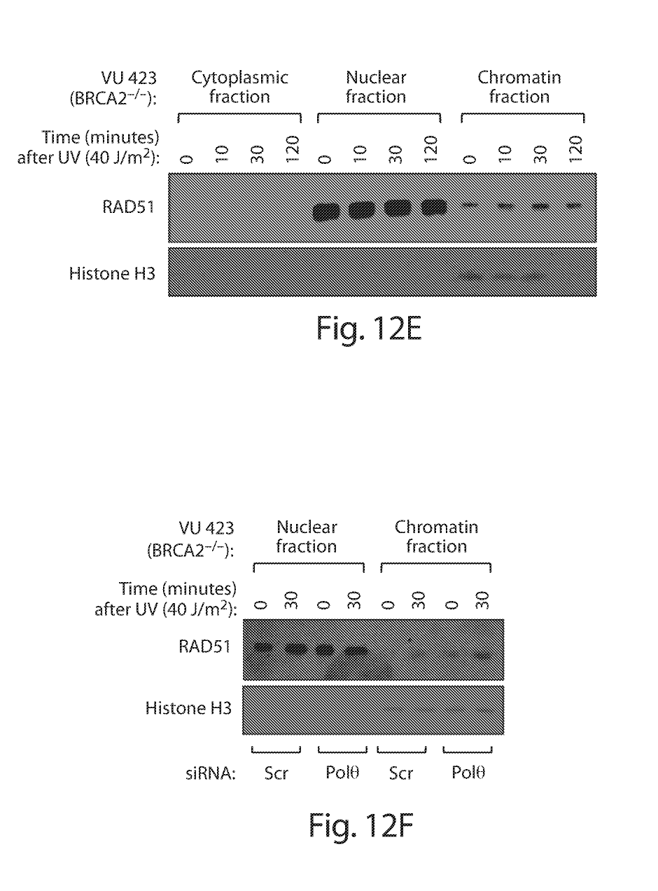

To understand which functions of POLQ are required for resistance to DNA-damaging agents, a series of complementation studies in HR-deficient cells was performed. Expression of full-length POLQ or ΔPol1, but not ARAD51, in HR-deficient POLQ-depleted cells treated with PARPi or MMC was able to rescue toxicity, suggesting that the anti-recombinase activity of POLQ maintains the genomic stability of HR-deficient cells (FIGS. 4G-4H and FIGS. 12A-12B). Moreover, the toxicity induced by loss of POLQ in HR-deficient cells was rescued by depletion of RAD51 showing that, in the absence of POLQ, RAD51 is toxic to HR-deficient cells (FIG. 4I). These results suggest a role for POLQ in limiting toxic HR events²³ (FIGS. 8C-8F) and may explain why HR-deficient cells overexpress and depend on an anti-recombinase for survival.

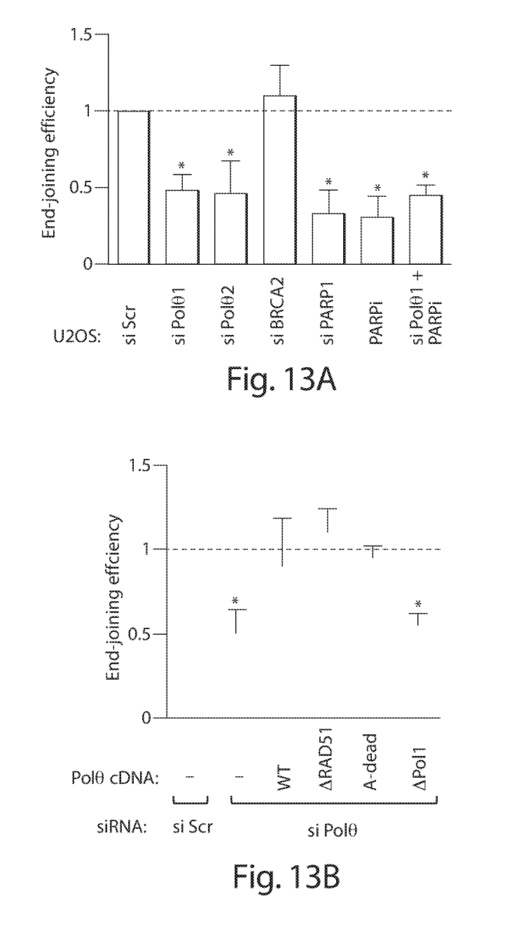

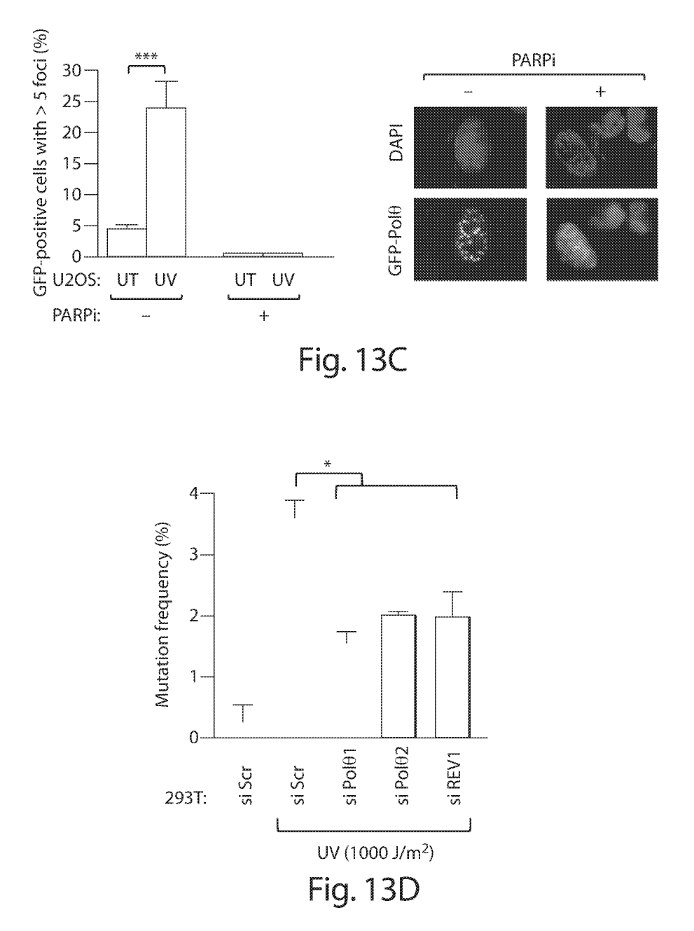

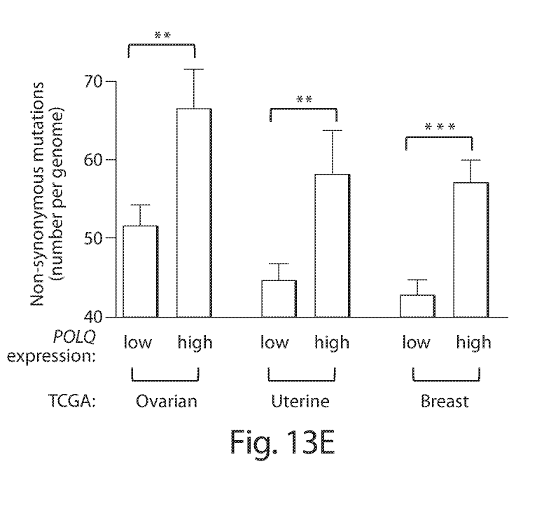

High mutation rates have been observed in HR-deficient tumors²⁴. Previous studies have shown that POLQ is an error-prone polymerase²⁵²⁶ that participates in alternative end-joining (alt-EJ)¹⁰. Therefore, the role of POLQ in error-prone DNA repair was assessed in human cancer cells. POLQ inhibition reduced alt-EJ efficiency in U2OS cells, similar to the reduction observed following depletion of PARP1, another critical factor in end-joining^{27,28} (FIG. 13A). Expression of full-length POLQ, ARAD51, or A-dead, but not the ΔPol1 mutant, complemented the cells, suggesting that the polymerase domain of POLQ is required for end-joining (FIG. 13B). GFP-tagged full-length POLQ formed foci after UV treatment in a PARP-dependent manner (FIG. 13C). POLQ inhibition reduced the mutation frequency induced by UV light, and tumors with high POLQ expression harbored more somatic point mutations than those with lower POLQ levels (FIGS. 13D-13E). These results suggest that POLQ contributes to the mutational signature observed in some HR-deficient tumors²⁹.

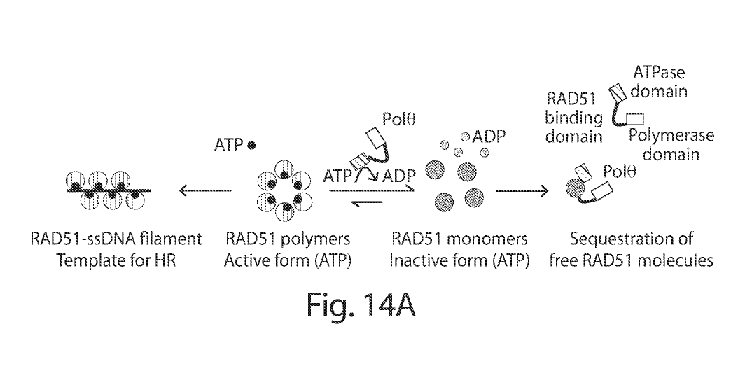

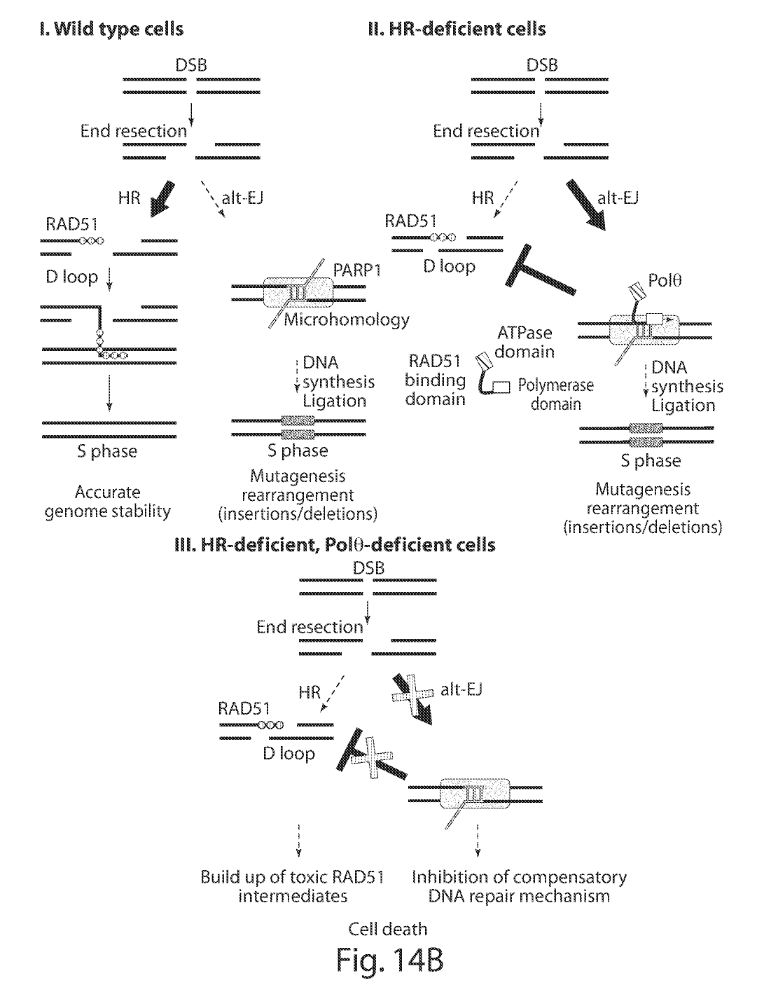

In human cancers, a deficiency in one DNA repair pathway can result in cellular hyper-dependence on a second compensatory DNA repair pathway⁴. POLQ is overexpressed in EOCs and other tumors with HR defects³⁰. Wild-type POLQ limits RAD51-ssDNA nucleofilament assembly (FIG. 14A) and promotes alt-EJ (FIG. 4J). HR-deficient tumors are hypersensitive to inhibition of POLQ-mediated repair. Therefore, POLQ appears to channel DNA repair by antagonizing HR and promoting PARP1-dependent error-prone repair (FIG. 14B). These results offer a potential new therapeutic target for cancers with inactivated HR.

### Materials and Methods

### Bioinformatic Analysis.

Gene Set Enrichment Analysis algorithm (GSEA, www.broadinstitute.org) was performed for the datasets. Gene sets are described below in Tables 3 and 4. Row expression data were downloaded from Gene Expression Omnibus (GEO). Quantile normalizations were performed using the RMA routine through GenePattern. GSEA was run using GenePattern (www.broadinstitute.org) and corresponding P values were computed using 2,000 permutations. The DNA repair gene set used in FIG. 5G has been determined according to a list of 151 DNA genes previously used³¹. GSEA analysis for 151 repair genes has been performed on the ovarian serous datasets (GSE14001, GSE14007, GSE18520, GSE16708, GSE10971). The list of 20 genes shown in FIG. 5G represents the top 20 expressed gene in cancer samples (median of the 5 datasets). The waterfall plot in FIG. 5H was generated as follows: the 20 genes defined in FIG. 5G were used as a gene set; GSEA for indicated data sets was performed and the nominal P values were plotted. Supervised analysis of gene expression for GSE9891 was performed with respect to differential expression that differentiated the third of tumors with highest POLQ expression from the 2 third with lowest POLQ levels. A list of the 200 most differentially expressed probe sets between the 2 groups with false discovery rate <0.05 was analyzed for biological pathways (hypergeometrical test; www.broadinstitute.org). TCGA datasets were accessed through the public TCGA data portal (www.tcga-data.nci.nih.gov). FIG. 3A reflects POLQ gene expression in the ovarian carcinoma dataset GSE9891, uterine carcinoma TCGA and breast carcinoma TCGA. Normalization of POLQ expression values across datasets was performed using z-score transformation. POLQ expression values were subdivided in subgroups reflecting the stage of the disease (for GSE9891: grade 3 ovarian serous carcinoma, n=143 compared to type 1 (grade 1) ovarian cancers, n=20; for uterine: serous like tumors, n=60 compared to the rest of the tumors, n=172; for breast: basal like breast carcinoma, n=80 compared to the rest of the tumors, n=421). Progression-free survival curves were generated by the Kaplan-Meier method and differences between survival curves were assessed for statistical significance with the log-rank test. In the absence of a clinically defined cutoff point for POLQ expression levels patients were divided into 2 groups: those with POLQ mRNA levels equal to or above the median (POLQ high group) and those with values below the median (POLQ low group). The correlation of POLQ was analyzed with outcome in each group. Patients with CCNE amplification (resistant to CDDP) were excluded from the analysis. For mutation count, data was accessed from tumors included in the TCGA datasets for which gene expression and whole-exome DNA sequencing was available. Data were accessed through the public TCGA data portal and the cBioPortal for Cancer Genomics (www.cbioportal.org). For each TCGA dataset, non-synonymous mutation count was assessed in tumors with the highest POLQ expression (top 33%) and compared to tumors with low POLQ expression (the remaining, 67%). In the uterine TCGA²⁰, all tumors were curated except the ultra and hyper-mutated group (i.e., POLE and MSI tumors). In the breast TCGA³², all tumors were analyzed. In the ovarian TCGA¹, tumors harboring molecular alterations (via mutation and epigenetic silencing) of the HR pathway were curated.

TABLE 3
Gene sets.

| Translesion Synthesis GeneSet | | | |
|---|---|---|---|
| Hugo gene symbols | Genes | Locus | Proteins |
| POLH | POLH | 6p21.1-p12 | polη |
| POLK | POLK | 5q13 | polκ |
| POLI | POLI | 18q21.1 | polι |
| REV1L | REV1 | 2q11.2 | rev1 |
| REV3L | REV3L | 6q21 | rev3L |
| MAD2L2 | REV7/MAD2B | 1p36.22 | MAD2B |
| PCNA | PCNA | 20p12 | PCNA |
| UBE2A | UBE2A/RAD6 | Xq24 | rad6 |
| RAD18 | RAD18 | 3p25.3 | rad18 |
| USP1 | USP1 | 1p32.1-p31.3 | usp1 |
| TP53 | TP53 | 17p13.1 | p53 |
| POLQ | POLQ | 3q13.33 | pol θ |

TABLE 4
Polymerase Gene Set

| Hugo gene symbols | Genes | Locus | Proteins |
|---|---|---|---|
| POLA | POLA1 | Xp22.1-p21.3 | polα |
| POLB | POLB | 8p11.21 | polβ |
| POLD | POLD1 | 19q13.33 | polδ |
| POLE | POLE | 12q24.33 | polε |
| POLH | POLH | 6p21.1-p12 | polη |
| POLI | POLI | 18q21.1 | polι |
| POLK | POLK | 5q13 | polκ |
| POLL | POLL | 10q24.32 | polλ |
| POLM | POLM | 7p14.1 | polμ |
| POLN | POLN/POL4P | 4p16.3 | polν |
| POLQ | POLQ | 3q13.33 | polθ |
| REV1L | REV1 | 2q11.2 | rev1 |
| REV3L | REV3L | 6q21 | rev3L |

### Plasmid Construction.

To facilitate subcloning, a silent mutation (A390A) was introduced into the POLQ gene sequence to remove the unique Xhol cutting site. Full-length or truncated POLQ cDNA were PCR-amplified and subcloned into pcDNA3-N-Flag, pFastBac-C-Flag, pOZ—C-Flag-HA, or GFP-C1 vectors to generate the various constructs. Point mutations and loop deletions were introduced by QuikChange II XL Site-Directed Mutagenesis Kit (Agilent Technologies) and confirmed by DNA sequencing. For POLQ rescue experiments (FIGS. 4G-4H and FIGS. 7C-7D), POLQ cDNA constructs resistant to siPOLQ1 were generated into the pOZ-C-Flag-HA vector and the construct were stably expressed in indicated cell line by retroviral transduction. The POLQ ATPase catalytically-dead mutant (A-dead) was generated by mutating the walker A and B motifs (K121A and D216A, E217A, respectively). pOZ-C-Flag-HA POLQ constructs were generated for retroviral transduction, and stable cells were selected using magnetic Dynabeads (Life Technologies) conjugated to the IL2R antibody (Millipore).

SiRNA and shRNA Sequence Information.

For siRNA-mediated knockdown, the following target sequences were used: POLQ (Qiagen POLQ_1 used as siPOLQ1 and Qiagen POLQ_6 used as siPOLQ2); BRCA1 (Qiagen BRCA1_13); PARP1 (Qiagen PARP1_6); REV1 (5′-CAGCGCAUCUGUGCCAAAGAA-TT-3′) (SEQ ID NO: 1); BRCA2 (5′-GAAGAAUGCAGGUUUAAUATT-3′) (SEQ ID NO: 2); BLM (5′-AUCAGCUAGAGGCGAUCAATT-3′) (SEQ ID NO: 3); FANCD2 (5′-GGAGAUUGAUGGUCUACUATT-3′) (SEQ ID NO: 4) and PARI (5′-AGGACACAUGUAAAGGGAUUGUCUATT-3′) (SEQ ID NO: 5). AllStars negative control siRNA (Qiagen) served as the negative control. ShRNAs targeting human FANCD2 was previously generated in the pTRIP/DU3-MND-GFP vector³³. ShRNAs targeting human POLQ (CGGGCCTCTTTAGATATAAAT, SEQ ID NO: 6), human BRCA2 (AAGAAGAATGCAGGTTTAATA, SEQ ID NO: 7) or Control (Scr, scramble) were generated in the pLKO-1 vector. POLQ (V2THS_198349) and non-silencing TRIPZ-RFP doxycycline-inducible shRNA were purchased from Open Biosystems. All shRNAs were transduced using lentivirus.

### Immunoblot Analysis, Fractionation and Pull-Down Assays.

Cells were lysed with 1% NP40 lysis buffer (1% NP40, 300 mM NaCl, 0.1 mM EDTA, 50 mM Tris [pH 7.5]) supplemented with protease inhibitor cocktail (Roche), resolved by NuPAGE (Invitrogen) gels, and transferred onto nitrocellulose membrane, followed by detection using the LAS-4000 Imaging system (GE Healthcare Life Sciences). For immunoprecipitation, cells were lysed with 300 mM NaCl lysis buffer, and the lysates were diluted to 150 mM NaCl before immunoprecipitation. Lysates were incubated with anti-Flag agarose resin (Sigma) followed by washes with 150 mM NaCl buffer. In vitro transcription and translation reactions were carried out using the TNT T7 Quick Coupled Transcription-Translation System (Promega). For cellular fractionation, cells were incubated with low salt permeabilization buffer (10 mM Tris [pH 7.3], 10 mM KCl 1.5 mM MgCl2) with protease inhibitor on ice for 20 minutes. Following centrifugation, nuclei were resuspended in 0.2 M HCl and the soluble fraction was neutralized with 1 M Tris-HCl [pH 8.0]. Nuclei were lysed in 150 mM NaCl and following centrifugation, the chromatin pellet was digested by micrococcal nuclease (Roche) for 5 minutes at room temperature. Recombinant GST-RAD51 and GST-PCNA fusion protein were expressed in BL21 strain and purified using glutathione-Sepharose beads (GE Healthcare) as previously described¹⁵. Beads with equal amount of GST or GST-RAD51 were incubated with in vitro-translated Flag-tagged POLQ variants in 150 mM NaCl lysis buffer.

### Antibodies and Chemicals.

Antibodies used in this study included: anti-PCNA (PC-10), anti-FANCD2 (FI-17), anti-RAD51 (H-92), anti-GST (B14), and Histone H3 (FL-136) and anti-vinculin (H-10) (Santa Cruz); anti-Flag (M2) (Sigma); anti-pS317CHK1 (2344), anti-pT68CHK2 (2661) (Cell signaling); anti-pS824KAP-1 (A300-767A) (Bethyl); anti-pS317γH2AX (05636) (Millipore); anti-pS15p53 (ab1431) and anti-POLQ (ab80906) (abcam); anti-BrdU (555627) (BD Pharmingen). Mitomycin C (MMC), cis-diamminedichloroplatinum(II) (Cisplatin, CDDP), and Hydroxyurea (HU) were purchased from Sigma. The PARPi rucaparib (AG-014699) was purchased from Selleckchem and ABT-888 from AbbVie. Rucaparib was used for all in vitro assays and ABT-888 was used for all in vivo experiments.

### Chromosomal Breakage Analysis.

293T and Vu 423 cells were twice-transfected with siRNAs for 48 hours and incubated for 48 hours with or without the indicated concentrations of MMC. For complementation studies on 293T shFANCD2, POLQ cDNA constructs were transfected 24 hours after the first siRNA transfection. Cells were exposed for 2 hours to 100 ng/ml of colcemid and treated with a hypotonic solution (0.075 M KCl) for 20 minutes and fixed with 3:1 methanol/acetic acid. Slides were stained with Wright's stain and 50 metaphase spreads were scored for aberrations. The relative number of chromosomal breaks was calculated relative to control cells (si Scr). For clarity of the FIG. 4B, radial figures were excluded from the analysis.

### Reporter Assays and Immunofluorescence.

HR and alt-EJ efficiency was measured using the DR-GFP (HR efficiency) and the alt-EJ reporter assay, performed as previously described^{14,27,34}. Briefly, 48 hours before transfection of SceI cDNA, U20S-DR-GFP cells were transfected with indicated siRNA or PARPi (1 μM). The HR activity was determined by FACS quantification of viable GFP-positive cells 96 hours after SceI was transfected. For RAD51 immunofluorescence experiments, cells were transfected with indicated siRNA 48 hours before treatment with HU (2 mM) or IR (10 Gy). For complementation studies, POLQ cDNA constructs were either transfected 24 hours after siRNA transfection (FIGS. 2B-2C and FIG. 9B) or stably expressed in indicated cell line (FIGS. 7C-7D). 6 hours after HU or IR treatment, cells were fixed with 4% paraformaldehyde for 10 minutes at room temperature, followed by extraction with 0.3% Triton X-100 for 10 minutes on ice. Antibody staining was performed at room temperature for 1 hour. For quantification of RAD51 foci in BrdU positive cells, cells were transfected with indicated siRNA 48 hours before treatment with IR (10 Gy). 2 hours after IR treatment, cells were treated with BrdU pulse (10 μM) for 2 hours and subsequently fixed with 4% paraformaldehyde and stained for RAD51 as described above. Cells were then fixed in ethanol (4° C., overnight), treated with 1.5 M HCL for 30 minutes and stained for BrdU antibody. The relative number of cells with more than 10 RAD51 foci was calculated relative to control cells (si Scr). Statistical differences between cells transfected with siRNAs (si POLQ1, si POLQ2, si BRCA2, si PARI or si BLM relative to control (si Scr) were assessed. For GFP fluorescence, cells were grown on coverslip, treated with UV (24 hours after GFP-POLQ transfection; 20 J/m²), fixed with 4% paraformaldehyde for 10 min at 25° C. 4 hours after the UV treatment, washed three times with PBS and mounted with DAPI-containing mounting medium (Vector Laboratories). When indicated cells were treated with PARPi (1 μM) 24 hours before GFP-POLQ transfection. Images were captured using a Zeiss AX10 fluorescence microscope and AxioVision software. Cells with GFP foci were quantified by counting number of cells with more than five foci. At least 150 cells were counted for each sample.

### Cell Survival Assays.

For assessing cellular cytotoxicity, cells were seeded into 96-well plates at a density of 1000 cells/well. Cytotoxic drugs were serially diluted in media and added to the wells. At 72 hours, CellTiter-Glo reagent (Promega) was added to the wells and the plates were scanned using a luminescence microplate reader. Survival at each drug concentration was plotted as a percentage of the survival in drug-free media. Each data point on the graph represents the average of three measurements, and the error bars represent the standard deviation. For clonogenic survival, 1000 cells/well were seeded into six-well plates and treated with cytotoxic drugs the next day. For MMC and PARPi, cells were treated continuously with indicated drug concentrations. For CDDP, cells were treated for 24 hours and cultured for 14 days in drug-free media. Colony formation was scored 14 days after treatment using 0.5% (w/v) crystal violet in methanol. Survival curves were expressed as a percentage ±s.e.m. over three independent experiments of colonies formed relative to the DMSO-treated control.

### Cell Cycle Analysis.

A2780 cells expressing Scr or POLQ shRNA were synchronized by a double thymidine block (Sigma) and subsequently exposed to MMC (1 μg/ml for 2 hours), IR (10 Gy) or HU (2 mM, overnight). At the indicated time points following drug release, cells were fixed in chilled 70% ethanol, stored overnight at −20° C., washed with PBS, and resuspended in propidium iodide. A fraction of those cells was analyzed by immunoblotting for DNA damage response proteins. The immunoblot analysis of γH2AX shows staining after 0, 24, 48 and 72 hours of HU treatment. For proliferation experiments, cells were incubated with 5-ethynyl-2′-deoxyuridine (EdU) (10 μM) for 1 hour at each time point after MMC exposure (1 μg/ml for 2 hours). Cells were washed and resuspended in culture medium for 2 hours prior to be analyzed by flow cytometry. Edu Staining was performed using the Click-iT EdU kit (Life Technologies).

### DNA Fiber Analysis.

A2780 cells expressing Scr or POLQ shRNA were incubated with 25 μM chlorodeoxyuridine (CldU) (Sigma, C6891) for 20 minutes. Cells were then treated with 2 mM hydroxyurea (HU) for 2 hours and incubated in 250 μM iododeoxyuridine (ldU) (Sigma, I7125) for 25 minutes after washout of the drug. Spreading of DNA fibers on glass slides was done as reported¹⁹. Glass slides were then washed in distilled water and in 2.5 M HCl for 80 minutes followed by three washes in PBS. The slides were incubated for 1 hour in blocking buffer (PBS with 1% BSA and 0.1% NP40) and then for 2 hours in rat anti-BrdU antibody (1:250, Abcam, ab6326). After washing with blocking buffer the slides were incubated for 2 hours in goat anti-rat Alexa 488 antibody (1:1000, Life Technologies, A-11006). The slides were then washed with PBS and 0.1% NP40 and then incubated for 2 hours with mouse anti-BrdU antibody diluted in blocking buffer (1:100, BD Biosciences, 347580). Following an additional wash with PBS and 0.1% NP40, the fibers were stained for 2 hours with chicken anti-mouse Alexa 594 (1:1000, Life Technologies, A-21201). At least 150 fibers were counted per condition. Pictures were taken with an Olympus confocal microscope and the fibers were analyzed by ImageJ software. The number of stalled or collapsed forks were measured by DNA fibers that had incorporated only CIdU. Stalled or collapsed forks counted in POLQ-depleted cells is expressed as fold-change after HU treatment relative to the fold-change observed in control cells, which was arbitrarily set to 1.

### SupF Mutagenesis Assay.

293T cells twice-transfected with siRNAs for 48 hours were then transfected with undamaged or damaged (UVC, 1,000 J/m²) pSP189 plasmids using GeneJuice (Novagen). After 48 hours, plasmid DNA was isolated with a miniprep kit (Promega) and digested with Dpnl. After ethanol precipitation, extracted plasmids were transformed into the β-galactosidase-MBM7070 indicator strain through electroporation (GenePulsor X Cell; Bio-Rad) and plated onto LB plates containing 1 mM IPTG, 100 m/ml 5-bromo-4-chloro-3-indolyl-β-D-galactopyranoside and 100 μg/ml ampicillin. White and blue colonies were scored using ImageJ software, and the mutation frequency was calculated as the ratio of white (mutant) to total (white plus blue) colonies.

### POLQ Gene Expression.

RNA samples extracted using the TRIzol Reagent (Invitrogen) were reverse transcribed using the Transcriptor Reverse Transcriptaze kit (Roche) and oligo dT primers. The resulting cDNA was use to analyzed POLQ expression by RT-qPCR using with QuantiTect SYBRGreen (Qiagen), in an iCycler machine (Bio-Rad). POLQ gene expression values were normalized to expression of the housekeeping gene GAPDH, using the ΔCT method and are shown on a log₂ scale. The primers used for POLQ are as follows: POLQ primer 1 (Forward: 5′-TATCTGCTGGAACTTTTGCTGA-3′ SEQ ID NO: 8; Reverse: 5′-CTCACACCATTTCTTTGATGGA-3′, SEQ ID NO: 9); POLQ primer 2 (Forward: 5′-CTACAAGTGAAGGGAGATGAGG-3′ SEQ ID NO: 10; Reverse: 5′-TCAGAGGGTTTCACCAATCC-3′, SEQ ID NO: 11).

POLQ Purification from Insect SF9 Cells.

A POLQ fragment (ΔPol2) containing the ATPase domain with a RAD51 binding site (amino acids 1 to 1000) was cloned into pFastBac-C-Flag and purified from baculovirus-infected SF9 insect cells as previously described³⁵. Briefly, SF9 cells were seeded in 15-cm dishes at 80-90% confluency and infected with baculovirus. Three days post-infection, cells were harvested and lysed in 500 mM NaCl lysis buffer (500 mM NaCl, 0.01% NP40, 0.2 mM EDTA, 20% Glycerol, 1 mM DTT, 0.2 mM PMSF, 20 mM Tris [pH 7.6]) supplemented with Halt protease inhibitor cocktail (Thermo Scientific) and Calpain I inhibitor (Roche) and the protein was eluted in lysis buffer supplemented with 0.2 mg/ml of Flag peptide (Sigma). The protein was concentrated in lysis buffer using 10 kDa centrifugal filters (Amicon). The protein was quantified by comparing its staining intensity (Coomassie-R250) with that of BSA standards in a 7% tris-glycine SDS-PAGE gel. Purified protein was flash-frozen in small aliquots in liquid nitrogen and stored at −80° C.

### Radiometric ATPase Assay.

Each 10 μl reaction consisted of 200 nM ATP, reaction buffer (20 mM Tris-HCl [pH 7.6], 5 mM MgCl₂, 0.05 mg/ml BSA, 1 mM DTT), and 5 μCi of [γ-³²P]-ATP. For corresponding reactions, ssDNA, dsDNA, and forked DNA were added to the reaction in excess at a final concentration of 600 nM. Once all of the non-enzymatic reagents were combined, recombinant POLQ was added to start the ATPase reaction. After incubation for 90 minutes at room temperature, stop buffer (125 mM EDTA [pH 8.0]) was added and approximately ˜0.05 μCi was spotted onto PEI-coated thin-layer chromatography (TLC) plates (Sigma). Unhydrolyzed [γ-³²P]-ATP was separated from the released inorganic phosphate [³²Pᵢ] with 1 M acetic acid, 0.25 M lithium chloride as the mobile phase. TLC plates were exposed to a phosphor screen and imaged with the BioRad Imager PMC. ssDNA, dsDNA, and forked DNA were generated as previously described³⁵. To remove any contaminating ssDNA, dsDNA and forked DNA were gel purified after annealing. Spots corresponding to [γ-³²P]-ATP and the released inorganic phosphate [³²Pᵢ] were quantified (in units of pixel intensity) and the fraction of ATP hydrolyzed calculated for each POLQ concentration.

### Electrophoretic Mobility Gel Shift Assay (EMSA).

Binding of POLQ to ssDNA was assessed using EMSA. 60-mer single-stranded DNA (ssDNA) or double-stranded DNA (dsDNA) oligonucleotides (5 nM) were incubated with increasing amount of POLQ (0, 5, 10, 50, or 100 nM) in 10 μl of binding buffer (20 mM HEPES-K+, [pH 7.6], 5 mM magnesium acetate, 0.1 μg/μl BSA, 5% glycerol, 1 mM DTT, 0.2 mM EDTA, and 0.01% NP-40) for one hour on ice. POLQ protein was added at a 10-fold dilution so that the final salt concentration was approximately 50 mM NaCl. The ssDNA probes are 5′ fluorescently-labeled with IRDye-700 (IDT). After incubation, the samples were analyzed on a 5% native polyacrylamide/0.5×TBE gel at 4° C. A fluorescent imager (Li-Cor) was used to visualize the samples in the gel.

### Rad51 Purification.

Human GST-RAD51 was purified from bacteria as described³⁶. Xenopus RAD51 (xRAD51) was purified as follow. N-terminally His-tagged SUMO-RAD51 was expressed in BL21 pLysS cells. Three hours after induction with 1 mM IPTG cells were harvested and resuspended in Buffer A (50 mM Tris-Cl [pH 7.5], 350 mM NaCl, 25% Sucrose, 5 mM β-mercaptoethanol, 1 mM PMSF and 10 mM imidazole). Cells were lysed by supplementation with Triton X-100 (0.2% final concentration), three freeze-thaw cycles and sonication (20 pulses at 40% efficiency). Soluble fraction was separated by centrifugation and incubated with 2 mL of Ni-NTA resin (Qiagen) for 1 hour at 4° C. After washing the resin with 100 mL of wash buffer (Buffer A supplemented with 1 M NaCl, final concentration) the salt concentration was brought down to 350 mM. His-SUMO-RAD51 was eluted with a linear gradient of imidazole from 10 mM-300 mM in Buffer A. Eluted fractions were analyzed by SDS-PAGE. His-SUMO-RAD51 containing fractions were pooled and supplemented with Ulp1 protease to cleave the His-SUMO tag and dialyzed overnight into Buffer B (50 mM Tris-Cl [pH 7.5], 350 mM NaCl, 25% Sucrose, 10% Glycerol, 5 mM β-mercaptoethanol, 10 mM imidazole and 0.05% Triton X-100). The dialyzed fraction was incubated with Ni-NTA resin for 1 hour at 4° C. and the RAD51 containing flow-through fraction was collected and dialyzed overnight into Buffer C (100 mM Potassium phosphate [pH 6.8], 150 mM NaCl, 10% Glycerol, 0.5 mM DTT and 0.01% Triton-X). RAD51 was further purified by Hydroxyapatite (Bio-Rad) chromatography. After washing with ten column volumes of Buffer C, RAD51 was eluted with a linear gradient of Potassium phosphate [pH 6.8] from 100 mM-800 mM. RAD51 containing fractions were analyzed by SDS-PAGE and dialyzed into storage buffer (20 mM HEPES-KOH [pH 7.4], 150 mM NaCl, 10% Glycerol, 0.5 mM DTT). Purified protein was flash-frozen in small aliquots in liquid nitrogen and stored at −80° C.

### D-Loop Assay.

D-loop formation assays were performed using xRAD51 and conducted as previously described³⁷. Briefly, nucleofilaments were first formed by incubating RAD51 (1 μM) with end-labeled 90-mer ssDNA (3 μM nt) at 37° C. for 10 minutes in reaction buffer containing 20 mM HEPES-KOH [pH 7.4], 1 mM ATP, 1 mM Mg(Cl)₂, 1 mM DTT, BSA (100 μg/mL), 20 mM phosphocreatine and creatine phosphokinase (20 μg/mL). After the 10 minutes incubation increasing amounts of POLQ (0, 0.1, 0.5, or 1.0 μM) and RPA (200 nM) were added and incubated for an additional 15 minutes at 37° C. Reaction was then supplemented with 1 mM CaCl₂ followed by further incubation at 37° C. for 15 minutes. D-loop formation was initiated by the addition of supercoiled dsDNA (pBS-KS (−), 79 μM bp) and incubation at 37° C. for 15 minutes. D-loops were analyzed by electrophoresis on a 0.9% agarose gel after deproteinization. Gel was dried and exposed to a Phospholmager (GE Healthcare) screen for quantification.

### Substitution Peptide Arrays and RAD51-ssDNA Filament Experiments.

Substitution peptide arrays were performed as previously described¹⁷. RAD51 displacement assays were performed as follow. Binding reactions (10 μl) contained 5′-32P-end-labelled DNA substrates (0.5 ng of 60 mer ssDNA) and various amounts of human RAD51 and/or POLQ in binding buffer (40 mM Tris-HCl [pH 7.5], 50 mM NaCl, 10 mM KCl, 2 mM DTT, 5 mM ATP, 5 mM MgCl2, 1 mM DTT, 100 mg/ml BSA) were conducted at room temperature. After 5 minutes incubation with POLQ and a further 5 minutes incubation with RAD51 or vice versa, an equimolar amount of cold DNA substrate was added to the reaction. Products were then analyzed by electrophoresis through 10% PAGE (200V for 40 min in 0.5×Tris-borate-EDTA buffer) and visualized by autoradiography.

### Interbreeding of the Fancd2 and Polq Mice.

For the characterization of Fancd2/Polq conditional knockouts, C57BL/6J mice (Jackson Laboratory) were crossed. Fancd2^{+/-}Polq^{+/+} mice, previously generated in our laboratory²², were crossed with Fancd2^{+/+}Polq^{+/-} mice⁷ to generate Fancd2^{+/-}Polq^{+/-} mice. These double heterozygous mice were then interbred, and the offspring from these mating pairs were genotyped using PCR primers for Fancd2 and Polq. A statistical comparison of the observed with the predicted genotypes was performed using a 2-sided Fisher's exact test. Primary MEFs were generated from E13.5 to E15 embryos and cultured in RPMI supplemented with 15% fetal bovine serum and 1% penicillin-streptomycin. All data generated in the study were extracted from experiments performed on primary MEFs from passage 1 to passage 4. The primers used for mice genotyping are as follows: Fancd2 PCR primers OST2cF (5′-CATGCATATAGGAACCCGAAGG-3′, SEQ ID NO: 12), OST2aR (5′-CAGGACCTTTGGAGAAGCAG-3′, SEQ ID NO: 13) and LTR2bF (5′-GGCGTTACTTAAGCTAGCTTG-3′, SEQ ID NO: 14); Polq PCR primers IMR5973 (5′-TGCAGTGTACAGATGTTACTTTT-3′, SEQ ID NO: 15), IMR 5974 (5′-TGGAGGTAGCATTTCTTCTC-3′, SEQ ID NO: 16), IMR 5975 (5′-TCACTAGGTTGGGGTTCTC-3′, (SEQ ID NO: 17) and IMR 5976 (5′-CATCAGAAGCTGACTCTAGAG-3′, (SEQ ID NO: 18).

### Studies of Xenograft-Bearing CrTac:NCr-Foxnlnu Mice.

The Animal Resource Facility at The Dana-Farber Cancer Institute approved all housing situations, treatments and experiments using mice. No more than five mice were housed per air-filtered cage with ad libitum access to standard diet and water, and were maintained in a temperature and light-controlled animal facility under pathogen-free conditions. All mice described in this text were drug and procedure naïve before the start of the experiments. For every xenograft study, approximately 1.0×10⁶ A2780 cells (1:1 in Matrigel Matrix, BD Biosciences) were subcutaneously implanted into both flanks of 6-8 week old female CrTac:NCr-Foxn1nu mice (Taconic). Doxycycline (Sigma) was added to the food (625 PPM) and bi-weekly (Tuesday and Friday) to the water (200 μg/ml) for mice bearing tumors that reached 100-200 mm³. Roughly one week (5-6 days) after the addition of Doxycycline to the diet, mice were randomized to twice daily treatment schedules with vehicle (0.9% NaCl) or PARPi (ABT-888; 50 mg per kg body weight) by oral gavage administration for the indicated number of weeks. Overall survival was determined using Kaplan-Meier analyses performed with Log-Rank tests to assess differences in median survival for each shRNA condition (shScr or shPOLQ) and each treatment condition (vehicle or PARPi) (GraphPad Prism 6 Software). For competition assays, A2780 cells expressing FANCD2-GFP shRNA (GFP cells) or a combination of FANCD2-GFP shRNA with (doxycycline inducible) Scr-RFP or POLQ-RFP shRNA (GFP-RFP cells) were mixed at an equal ratio of GFP to GFP-RFP cells, and thereafter injected into nude mice given doxycycline-containing diets and treated with either vehicle or PARPi or CDDP. For competition assays, mice received identical doxycycline and PARPi drug treatment. For the Cisplatin competition assay, mice were randomized into semi-weekly treatment regimens with vehicle (0.9% NaCl) or CDDP (5 mg per kg body weight) by intraperitoneal injection. After three to four weeks of treatment, mice were euthanized and tumors were grown in vitro, in the presence of doxycycline (2 μg/ml for 4 days). The relative ratio of GFP to GFP-RFP cells was determined by FACS analysis. Tumor volumes were calculated bi-weekly using caliper measurements (length×width)/2. Growth curves were plotted as the mean tumor volume (mm³) for each treatment group; relative tumor volume (RTV) indicates change in tumor volume at a given time point relative to that at the day before initial dosing (=1). Mice were unbiasedly assigned into different treatment groups. Drug treatment and outcome assessment was performed in a blinded manner. Mice were monitored every day and euthanized by CO₂ inhalation when tumor size (≥2 cm), tumor status (necrosis/ulceration) or body weight loss (≥20%) reached ethical endpoint, according to the rules of the Animal Resource Facility at The Dana-Farber Cancer Institute.

### Immunohistochemical Staining.

Formalin-fixed paraffin-embedded sections of harvested xenografts were stained with antibodies specific for γ-H2AX (pSer139) (Upstate Biotechnology) and Ki67 (Dako). At least two xenografts were scored for each treatment. Tumors were collected three weeks after treatment. At least five 40× fields were scored. The mean±s.e.m. percentage of positive cells from five images in each treatment group was calculated.

### Statistical Analysis.

Unless stated otherwise, all data are represented as mean±s.e.m. over at least three independent experiments, and significance was calculated using the Student's t test. Asterisks indicate statistically significant (*, P<0.05; **, P<10⁻²; ***, P<10⁻³) values. All the in vivo experiments were run with at least 6 tumors from 6 mice for each condition.

### Example 2: Screening Methods

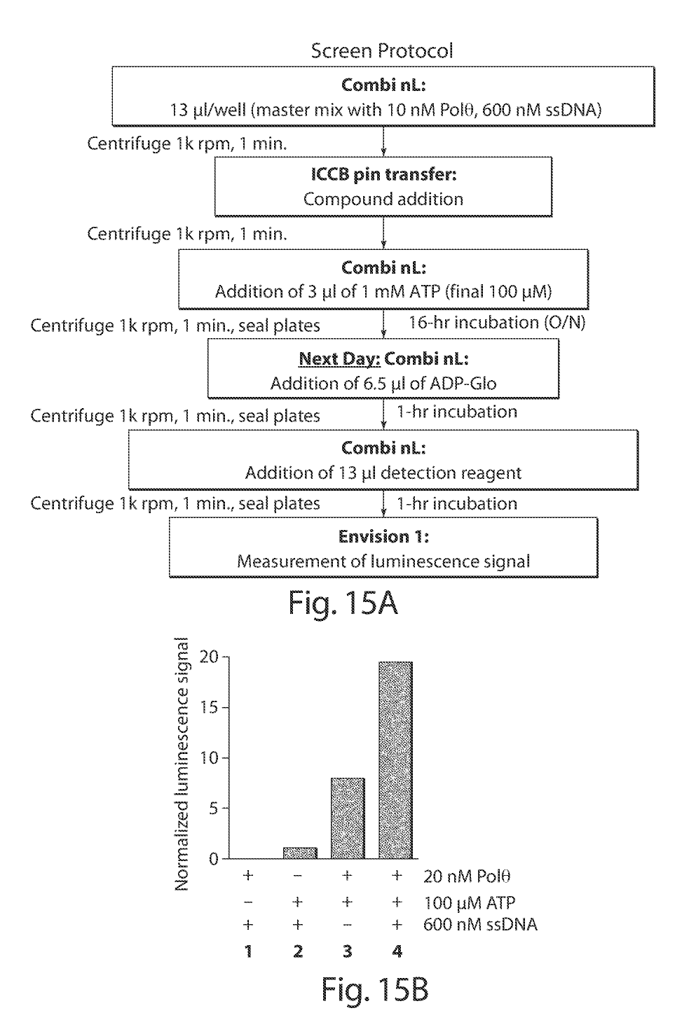

High-throughput screening for inhibitors of the ATPase activity of Polθ was conducted in 384-well low-volume plates (Corning). The ADP-Glo™ kinase assay kit (Promega, V9103) was used to detect ATPase activity. Briefly, reactions contained a single-stranded 30-mer DNA substrate (600 nM), recombinant Polθ-ΔPol2 ((10 nM), −/+ small-molecule compound or DMSO, and pure ATP (from kit, 100 μM). After an overnight incubation of the sealed 384-well plates for ˜16 hours, ADP-Glo™ reagent was (Promega kit, V9103) added, plates were incubated for one hour, the detection reagent (Promega kit, V9103) added followed by another one-hour incubation, and the luminescence signal read using a plate reader (EnVision). All steps were performed at room temperature. FIG. 15A shows a flowchart depicting one embodiment of the screening method. FIG. 15B shows characterization of the ATP hydrolysis activity of purified Polθ fragment using the ADP-Glo™ kinase assay.

### Example 3: Polθ Expression in Suspension

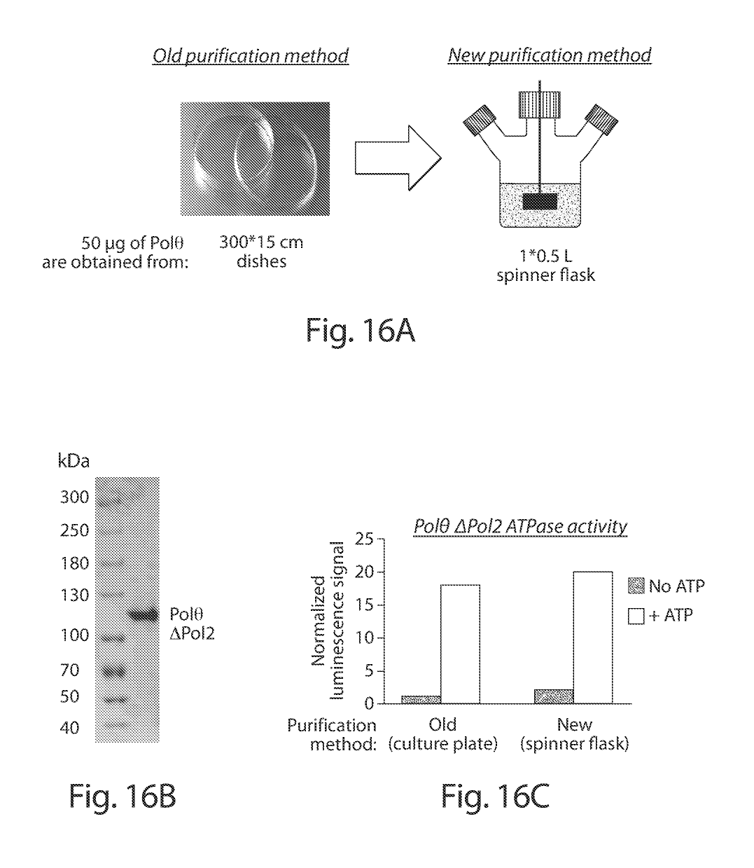

A culture plate-based protein purification method was adapted to a spinner flask culture system to obtain purified Polθ (ΔPol2) (FIG. 16A-16B). Polθ (ΔPol2) pFastbac I plasmid DNA was transformed into DH10Bac competent cells. The transformed cells were plated and incubated until colonies were distinguishable. A colony was picked, inoculated into a liquid culture, and grown overnight. Bacmid DNA was subsequently purified from cells in the cultured medium.

To obtain a first amplification of baculovirus, SF9 cells were seeded in a plate with insect cell media and allowed to attach overnight. Purified bacmid DNA was mixed with CellFECTIN II Reagent and added to the plate to transfect SF9 cells. Following an incubation period, transfected SF9 cells were pelleted and supernatant containing the first amplification of baculovirus was collected. To obtain a second amplification of baculovirus, fresh SF9 cells seeded in a tissue culture plate were infected with the first amplification of baculovirus. Following incubation, the second amplification of baculovirus was isolated.

Fresh SF9 cells were grown in suspension culture using a spinner flask, and baculovirus was added to the flask to infect SF9 cells. Following incubation, infected SF9 cells were lysed and Polθ (ΔPol2) was purified from the lysate. Polθ (ΔPol2) purified using the spinner flask purification system exhibited levels of enzymatic activity comparable to that of Polθ (ΔPol2) purified using a culture plate-based purification system (FIG. 16C).

### REFERENCES

1 Cancer Genome Atlas Research, N. Integrated genomic analyses of ovarian carcinoma. Nature 474, 609-615, doi:10.1038/nature10166 (2011).
2 Farmer, H. et al. Targeting the DNA repair defect in BRCA mutant cells as a therapeutic strategy. Nature 434, 917-921, doi:10.1038/nature03445 (2005).
3 Bryant, H. E. et al. Specific killing of BRCA2-deficient tumours with inhibitors of poly(ADP-ribose) polymerase. Nature 434, 913-917, doi:10.1038/nature03443 (2005).
4 Kennedy, R. D. & D'Andrea, A. D. DNA repair pathways in clinical practice: lessons from pediatric cancer susceptibility syndromes. Journal of clinical oncology: official journal of the American Society of Clinical Oncology 24, 3799-3808, doi:10.1200/JCO.2005.05.4171 (2006).
5 Bast, R. C., Jr., Hennessy, B. & Mills, G. B. The biology of ovarian cancer: new opportunities for translation. Nature reviews. Cancer 9, 415-428, doi:10.1038/nrc2644 (2009).
6 Yousefzadeh, M. J. & Wood, R. D. DNA polymerase POLQ and cellular defense against DNA damage. DNA repair 12, 1-9, doi:10.1016/j.dnarep.2012.10.004 (2013).
7 Shima, N., Munroe, R. J. & Schimenti, J. C. The mouse genomic instability mutation chaosl is an allele of Polq that exhibits genetic interaction with Atm. Molecular and cellular biology 24, 10381-10389, doi:10.1128/MCB.24.23.10381-10389.2004 (2004).
8 Yoshimura, M. et al. Vertebrate POLQ and POLbeta cooperate in base excision repair of oxidative DNA damage. Molecular cell 24, 115-125, doi:10.1016/j.molce1.2006.07.032 (2006).
9 Muzzini, D. M., Plevani, P., Boulton, S. J., Cassata, G. & Marini, F. Caenorhabditis elegans POLQ-1 and HEL-308 function in two distinct DNA interstrand cross-link repair pathways. DNA repair 7, 941-950, doi:10.1016/j.dnarep.2008.03.021 (2008).
10 McVey, M. & Lee, S. E. MMEJ repair of double-strand breaks (director's cut): deleted sequences and alternative endings. Trends in genetics: TIG 24, 529-538, doi:10.1016/j.tig.2008.08.007 (2008).
11 Chan, S. H., Yu, A. M. & McVey, M. Dual roles for DNA polymerase theta in alternative end-joining repair of double-strand breaks in Drosophila. PLoS genetics 6, e1001005, doi:10.1371/journal.pgen.1001005 (2010).
12 Yu, A. M. & McVey, M. Synthesis-dependent microhomology-mediated end joining accounts for multiple types of repair junctions. Nucleic acids research 38, 5706-5717, doi:10.1093/nar/gkq379 (2010).
13 Koole, W. et al. A Polymerase Theta-dependent repair pathway suppresses extensive genomic instability at endogenous G4 DNA sites. Nature communications 5, 3216, doi:10.1038/ncomms4216 (2014).
14 Nakanishi, K. et al. Human Fanconi anemia monoubiquitination pathway promotes homologous DNA repair. Proceedings of the National Academy of Sciences of the United States of America 102, 1110-1115, doi:10.1073/pnas.0407796102 (2005).
15 Moldovan, G. L. et al. Inhibition of homologous recombination by the PCNA-interacting protein PARI. Molecular cell 45, 75-86, doi:10.1016/j.molcel.2011.11.010 (2012).
16 Ira, G., Malkova, A., Liberi, G., Foiani, M. & Haber, J. E. Srs2 and Sgsl-Top3 suppress crossovers during double-strand break repair in yeast. Cell 115, 401-411 (2003).
17 Ward, J. D. et al. Overlapping mechanisms promote postsynaptic RAD-51 filament disassembly during meiotic double-strand break repair. Molecular cell 37, 259-272, doi:10.1016/j.molcel.2009.12.026 (2010).
18 Seki, M., Marini, F. & Wood, R. D. POLQ (Pol theta), a DNA polymerase and DNA-dependent ATPase in human cells. Nucleic acids research 31, 6117-6126 (2003).
19 Jackson, D. A. & Pombo, A. Replicon clusters are stable units of chromosome structure: evidence that nuclear organization contributes to the efficient activation and propagation of S phase in human cells. The Journal of cell biology 140, 1285-1295 (1998).
20 Cancer Genome Atlas Research, N. et al. Integrated genomic characterization of endometrial carcinoma. Nature 497, 67-73, doi:10.1038/nature12113 (2013).
21 Morrison, C. et al. The controlling role of ATM in homologous recombinational repair of DNA damage. The EMBO journal 19, 463-471, doi:10.1093/emboj/19.3.463 (2000).
22 Parmar, K. et al. Hematopoietic stem cell defects in mice with deficiency of Fancd2 or Uspl. Stem cells 28, 1186-1195, doi:10.1002/stem.437 (2010).
23 Hu, Y. et al. PARP1-driven Poly-ADP-ribosylation Regulates BRCA1 Function in Homologous Recombination Mediated DNA Repair. Cancer discovery, doi:10.1158/2159-8290.CD-13-0891 (2014).
24 Yang, D. et al. Association of BRCA1 and BRCA2 mutations with survival, chemotherapy sensitivity, and gene mutator phenotype in patients with ovarian cancer. JAMA: the journal of the American Medical Association 306, 1557-1565, doi:10.1001/jama.2011.1456 (2011).
25 Seki, M. et al. High-efficiency bypass of DNA damage by human DNA polymerase Q. The EMBO journal 23, 4484-4494, doi:10.1038/sj.emboj.7600424 (2004).
26 Seki, M. & Wood, R. D. DNA polymerase theta (POLQ) can extend from mismatches and from bases opposite a (6-4) photoproduct. DNA repair 7, 119-127, doi:10.1016/j.dnarep.2007.08.005 (2008).
27 Zhang, Y. & Jasin, M. An essential role for CtIP in chromosomal translocation formation through an alternative end-joining pathway. Nature structural & molecular biology 18, 80-84, doi:10.1038/nsmb.1940 (2011).
28 Chiruvella, K. K., Liang, Z. & Wilson, T. E. Repair of double-strand breaks by end joining. Cold Spring Harbor perspectives in biology 5, a012757, doi:10.1101/cshperspect.a012757 (2013).
29 Alexandrov, L. B. et al. Signatures of mutational processes in human cancer. Nature 500, 415-421, doi:10.1038/nature12477 (2013).
30 Lemee, F. et al. DNA polymerase theta up-regulation is associated with poor survival in breast cancer, perturbs DNA replication, and promotes genetic instability. Proceedings of the National Academy of Sciences of the United States of America 107, 13390-13395, doi:10.1073/pnas.0910759107 (2010).
31 Kang, J., D'Andrea, A. D. & Kozono, D. A DNA repair pathway-focused score for prediction of outcomes in ovarian cancer treated with platinum-based chemotherapy. Journal of the National Cancer Institute 104, 670-681, doi:10.1093/jnci/djs177 (2012).
32 Cancer Genome Atlas, N. Comprehensive molecular portraits of human breast tumours. Nature 490, 61-70, doi:10.1038/nature11412 (2012).
33 Ceccaldi, R. et al. Bone marrow failure in Fanconi anemia is triggered by an exacerbated p53/p21 DNA damage response that impairs hematopoietic stem and progenitor cells. Cell stem cell 11, 36-49, doi:10.1016/j.stem.2012.05.013 (2012).
34 Bennardo, N., Cheng, A., Huang, N. & Stark, J. M. Alternative-NHEJ is a mechanistically distinct pathway of mammalian chromosome break repair. PLoS genetics 4, el000110, doi:10.1371/journal.pgen.1000110 (2008).
35 Yusufzai, T. & Kadonaga, J. T. HARP is an ATP-driven annealing helicase. Science 322, 748-750, doi:10.1126/science.1161233 (2008).
36 Barber, L. J. et al. RTEL1 maintains genomic stability by suppressing homologous recombination. Cell 135, 261-271, doi:10.1016/j.cell.2008.08.016 (2008).
37 Amunugama, R., Groden, J. & Fishel, R. The HsRAD51B-HsRAD51C stabilizes the HsRAD51 nucleoprotein filament. DNA repair 12, 723-732, doi:10.1016/j.dnarep.2013.05.005 (2013).
38 Pitroda, S. P. et al. DNA repair pathway gene expression score correlates with repair proficiency and tumor sensitivity to chemotherapy. Science translational medicine 6, 229ra242, doi:10.1126/scitranslmed.3008291 (2014).
39 Aggarwal, M., Sommers, J. A., Shoemaker, R. H. & Brosh, R. M., Jr. Inhibition of helicase activity by a small molecule impairs Werner syndrome helicase (WRN) function in the cellular response to DNA damage or replication stress. Proceedings of the National Academy of Sciences of the United States of America 108, 1525-1530, doi:10.1073/pnas.1006423108 (2011).

We claim:

1. A method for treating homologous recombination (HR)-deficient cancer in a subject, the method comprising:
administering to the subject in need thereof a DNA polymerase θ (Polθ) inhibitor in an amount effective to treat the HR-deficient cancer.

2. The method of claim 1, further comprising treating the subject with one or more anti-cancer therapy.

3. The method of claim 2, wherein the anti-cancer therapy is selected from the group consisting of surgery, radiation therapy, chemotherapy, gene therapy, DNA therapy, viral therapy, RNA therapy, adjuvant therapy, and immunotherapy.

4. The method of claim 3, wherein the chemotherapy comprises administering to the subject a cytotoxic agent in an amount effective to treat the HR-deficient cancer.

5. The method of any one of claims 2-4, wherein the Polθ inhibitor and the anti-cancer therapy are synergistic in treating the cancer, compared to the Polθ inhibitor alone or the anti-cancer therapy alone.

6. The method of any one of claims 1-5, wherein the Polθ inhibitor is a small molecule, antibody, peptide or antisense compound.

7. The method of any one of claims 4-6, wherein the cytotoxic agent is selected from the group consisting of a platinum agent, mitomycin C, a poly (ADP-ribose) polymerase (PARP) inhibitor, a radioisotope, a vinca alkaloid, an antitumor alkylating agent, a monoclonal antibody and an antimetabolite.

8. The method of any one of claims 2-7, wherein the Polθ inhibitor and the anti-cancer therapy are administered concurrently or sequentially.

9. The method of any one of claims 1-8, wherein the HR-deficient cancer is resistant to treatment with a PARP inhibitor alone.

10. A method for treating a cancer that is resistant to PARP inhibitor therapy in a subject, the method comprising:
administering to the subject in need thereof a Polθ inhibitor in an amount effective to treat the PARP inhibitor-resistant cancer.

11. The method of claim 10, further comprising treating the subject with one or more anti-cancer therapy.

12. The method of claim 11, wherein the anti-cancer therapy is selected from the group consisting of surgery, radiation therapy, chemotherapy, gene therapy, DNA therapy, viral therapy, RNA therapy, adjuvant therapy, and immunotherapy.

13. The method of claim 12, wherein the chemotherapy comprises administering to the subject a cytotoxic agent in an amount effective to treat the HR-deficient cancer.

14. The method of any one of claims 11-13, wherein the Polθ inhibitor and the anti-cancer therapy are synergistic in treating the cancer, compared to the Polθ inhibitor alone or the anti-cancer therapy alone.

15. The method of any one of claims 10-14, wherein the Polθ inhibitor is a small molecule, antibody, peptide or antisense compound.

16. The method of any one of claims 13-15, wherein the cytotoxic agent is selected from the group consisting of a platinum agent, mitomycin C, a radioisotope, a vinca alkaloid, an antitumor alkylating agent, a monoclonal antibody and an antimetabolite.

17. The method of any one of claims 11-16, wherein the Polθ inhibitor and the anti-cancer therapy are administered concurrently or sequentially.

18. The method of any one of claims 10-17, wherein the PARP inhibitor-resistant cancer is deficient in homologous recombination.

19. A method for treating a cancer that is characterized by overexpression of Polθ in a subject, the method comprising
administering to the subject in need thereof a Polθ inhibitor in an amount effective to treat the Polθ-overexpressing cancer.

20. The method of claim 19, further comprising treating the subject with one or more anti-cancer therapy.

21. The method of claim 20, wherein the anti-cancer therapy is selected from the group consisting of surgery, radiation therapy, chemotherapy, gene therapy, DNA therapy, viral therapy, RNA therapy, adjuvant therapy, and immunotherapy.

22. The method of claim 21, wherein the chemotherapy comprises administering to the subject a cytotoxic agent in an amount effective to treat the HR-deficient cancer.

23. The method of any one of claims 20-22, wherein the Polθ inhibitor and the anti-cancer therapy are synergistic in treating the cancer, compared to the Polθ inhibitor alone or the anti-cancer therapy alone.

24. The method of any one of claims 19-23, wherein the Polθ inhibitor is a small molecule, antibody, peptide or antisense compound.

25. The method of any one of claims 22-24, wherein the cytotoxic agent is selected from the group consisting of a platinum agent, mitomycin C, a poly (ADP-ribose) polymerase (PARP) inhibitor, a radioisotope, a vinca alkaloid, an antitumor alkylating agent, a monoclonal antibody and an antimetabolite.

26. The method of any one of claims 20-25, wherein the Polθ inhibitor and the anti-cancer therapy are administered concurrently or sequentially.

27. The method of any one of claims 19-26, wherein the Polθ-overexpressing cancer is deficient in homologous recombination.

28. A method for treating a cancer that is characterized by one or more BRCA mutations and/or reduced expression of Fanconi (Fanc) proteins in a subject, the method comprising
administering to the subject in need thereof a Polθ inhibitor in an amount effective to treat the cancer.

29. The method of claim 28, further comprising treating the subject with one or more anti-cancer therapy.

30. The method of claim 29, wherein the anti-cancer therapy is selected from the group consisting of surgery, radiation therapy, chemotherapy, gene therapy, DNA therapy, viral therapy, RNA therapy, adjuvant therapy, and immunotherapy.

31. The method of claim 30, wherein the chemotherapy comprises administering to the subject a cytotoxic agent in an amount effective to treat the HR-deficient cancer.

32. The method of any one of claims 29-31, wherein the Polθ inhibitor and the anti-cancer therapy are synergistic in treating the cancer, compared to the Polθ inhibitor alone or the anti-cancer therapy alone.

33. The method of any one of claims 28-32, wherein the Polθ inhibitor is a small molecule, antibody, peptide or antisense compound.

34. The method of any one of claims 31-33, wherein the cytotoxic agent is selected from the group consisting of a platinum agent, mitomycin C, a PARP inhibitor, a radioisotope, a vinca alkaloid, an antitumor alkylating agent, a monoclonal antibody and an antimetabolite.

35. The method of any one of claims 29-34, wherein the Polθ inhibitor and the anti-cancer therapy are administered concurrently or sequentially.

36. The method of any one of claims 28-35, wherein the cancer is also characterized by overexpression of Polθ.

37. A high-throughput screening method for identifying an inhibitor of ATPase activity of Polθ, the method comprising:
(i) contacting Polθ or a fragment thereof with adenosine triphosphate (ATP) and single-stranded DNA (ssDNA) substrate in the presence and absence of a candidate compound;
(ii) quantifying amount of adenosine diphosphate (ADP) produced in the presence and absence of the candidate compound; and
(iii) identifying the candidate compound as an inhibitor of the ATPase activity of Polθ if the amount of ADP produced in the presence of the candidate compound is less than the amount produced in the absence of candidate compound.

38. The method of claim 37, wherein the amount of ADP produced is quantified using luminescence or radioactivity.

39. The method of any one of claims 37-38, wherein the amount of ADP is quantified using the ADP-Glo™ Kinase assay.

40. The method of claim 39, wherein the Polθ or fragment thereof, ATP and ssDNA substrate are incubated in the presence or absence of the candidate compound for at least 2 hours, 4 hours, 8 hours, 10 hours, 12 hours, 14 hours, 16 hours, or 18 hours.

41. The method of any one of claims 39-40, wherein 5 nM, 10 nM, or 15 nM of Polθ or a fragment thereof is used in step (i).

42. The method of any one of claims 39-41, wherein 25, 50, 100, 125, 150, or 175 μM of ATP is used in step (i).

43. The method of any one of claims 37-42, wherein the Polθ fragment comprises N-terminal ATPase domain of Polθ.

44. The method of any one of claims 37-43, wherein the candidate compound is a small molecule, antibody, peptide or antisense compound


SEQUENCE LISTING (74 sequences)

SEQ ID NO: 1 - DNA, 21 bp, Artificial Sequence
  Synthetic Polynucleotide
cagcgcaucugugccaaagaa

SEQ ID NO: 2 - DNA, 21 bp, Artificial Sequence
  Synthetic Polynucleotide
gaagaaugcagguuuaauatt

SEQ ID NO: 3 - DNA, 21 bp, Artificial Sequence
  Synthetic Polynucleotide
aucagcuagaggcgaucaatt

SEQ ID NO: 4 - DNA, 21 bp, Artificial Sequence
  Synthetic Polynucleotide
ggagauugauggucuacuatt

SEQ ID NO: 5 - DNA, 27 bp, Artificial Sequence
  Synthetic Polynucleotide
aggacacauguaaagggauugucuatt

SEQ ID NO: 6 - DNA, 21 bp, Artificial Sequence
  Synthetic Polynucleotide
cgggcctctttagatataaat

SEQ ID NO: 7 - DNA, 21 bp, Artificial Sequence
  Synthetic Polynucleotide
aagaagaatgcaggtttaata

SEQ ID NO: 8 - DNA, 22 bp, Artificial Sequence
  Synthetic Polynucleotide
tatctgctggaacttttgctga

SEQ ID NO: 9 - DNA, 22 bp, Artificial Sequence
  Synthetic Polynucleotide
ctcacaccatttctttgatgga

SEQ ID NO: 10 - DNA, 22 bp, Artificial Sequence
  Synthetic Polynucleotide
ctacaagtgaagggagatgagg

SEQ ID NO: 11 - DNA, 20 bp, Artificial Sequence
  Synthetic Polynucleotide
tcagagggtttcaccaatcc

SEQ ID NO: 12 - DNA, 22 bp, Artificial Sequence
  Synthetic Polynucleotide
catgcatataggaacccgaagg

SEQ ID NO: 13 - DNA, 20 bp, Artificial Sequence
  Synthetic Polynucleotide
caggacctttggagaagcag

SEQ ID NO: 14 - DNA, 21 bp, Artificial Sequence
  Synthetic Polynucleotide
ggcgttacttaagctagcttg

SEQ ID NO: 15 - DNA, 23 bp, Artificial Sequence
  Synthetic Polynucleotide
tgcagtgtacagatgttactttt

SEQ ID NO: 16 - DNA, 20 bp, Artificial Sequence
  Synthetic Polynucleotide
tggaggtagcatttcttctc

SEQ ID NO: 17 - DNA, 19 bp, Artificial Sequence
  Synthetic Polynucleotide
tcactaggttggggttctc

SEQ ID NO: 18 - DNA, 21 bp, Artificial Sequence
  Synthetic Polynucleotide
catcagaagctgactctagag

SEQ ID NO: 19 - protein, 20 aa, Artificial Sequence
  Synthetic Polypeptide
EEEAVEERRNMRTIWVTGRK

SEQ ID NO: 20 - protein, 20 aa, Artificial Sequence
  Synthetic Polypeptide
EEAVEERRNMRTIWVTGRKG

SEQ ID NO: 21 - protein, 20 aa, Artificial Sequence
  Synthetic Polypeptide
EAVEERRNMRTIWVTGRKGL

SEQ ID NO: 22 - protein, 20 aa, Artificial Sequence
  Synthetic Polypeptide
AVEERRNMRTIWVTGRKGLT

SEQ ID NO: 23 - protein, 20 aa, Artificial Sequence
  Synthetic Polypeptide
VEERRNMRTIWVTGRKGLTE

SEQ ID NO: 24 - protein, 20 aa, Artificial Sequence
  Synthetic Polypeptide
EERRNMRTIWVTGRKGLTER

SEQ ID NO: 25 - protein, 20 aa, Artificial Sequence
  Synthetic Polypeptide
ERRNMRTIWVTGRKGLTERE

SEQ ID NO: 26 - protein, 20 aa, Artificial Sequence
  Synthetic Polypeptide
RRNMRTIWVTGRKGLTEREA

SEQ ID NO: 27 - protein, 20 aa, Artificial Sequence
  Synthetic Polypeptide
RNMRTIWVTGRKGLTEREAA

SEQ ID NO: 28 - protein, 20 aa, Artificial Sequence
  Synthetic Polypeptide
NMRTIWVTGRKGLTEREAAA

SEQ ID NO: 29 - protein, 20 aa, Artificial Sequence
  Synthetic Polypeptide
MRTIWVTGRKGLTEREAAAL

SEQ ID NO: 30 - protein, 20 aa, Artificial Sequence
  Synthetic Polypeptide
RTIWVTGRKGLTEREAAALI

SEQ ID NO: 31 - protein, 20 aa, Artificial Sequence
  Synthetic Polypeptide
TIWVTGRKGLTEREAAALIV

SEQ ID NO: 32 - protein, 20 aa, Artificial Sequence
  Synthetic Polypeptide
NFLNISRLQEKTGTYTTNKT

SEQ ID NO: 33 - protein, 20 aa, Artificial Sequence
  Synthetic Polypeptide
FLNISRLQEKTGTYTTNKTK

SEQ ID NO: 34 - protein, 20 aa, Artificial Sequence
  Synthetic Polypeptide
LNISRLQEKTGTYTTNKTKN

SEQ ID NO: 35 - protein, 20 aa, Artificial Sequence
  Synthetic Polypeptide
NISRLQEKTGTYTTNKTKNN

SEQ ID NO: 36 - protein, 20 aa, Artificial Sequence
  Synthetic Polypeptide
ISRLQEKTGTYTTNKTKNNH

SEQ ID NO: 37 - protein, 20 aa, Artificial Sequence
  Synthetic Polypeptide
SRLQEKTGTYTTNKTKNNHV

SEQ ID NO: 38 - protein, 20 aa, Artificial Sequence
  Synthetic Polypeptide
RLQEKTGTYTTNKTKNNHVS

SEQ ID NO: 39 - protein, 20 aa, Artificial Sequence
  Synthetic Polypeptide
LQEKTGTYTTNKTKNNHVSD

SEQ ID NO: 40 - protein, 20 aa, Artificial Sequence
  Synthetic Polypeptide
QEKTGTYTTNKTKNNHVSDL

SEQ ID NO: 41 - protein, 20 aa, Artificial Sequence
  Synthetic Polypeptide
EKTGTYTTNKTKNNHVSDLG

SEQ ID NO: 42 - protein, 20 aa, Artificial Sequence
  Synthetic Polypeptide
KTGTYTTNKTKNNHVSDLGL

SEQ ID NO: 43 - protein, 20 aa, Artificial Sequence
  Synthetic Polypeptide
TGTYTTNKTKNNHVSDLGLV

SEQ ID NO: 44 - protein, 20 aa, Artificial Sequence
  Synthetic Polypeptide
GTYTTNKTKNNHVSDLGLVL

SEQ ID NO: 45 - protein, 20 aa, Artificial Sequence
  Synthetic Polypeptide
TYTTNKTKNNHVSDLGLVLC

SEQ ID NO: 46 - protein, 20 aa, Artificial Sequence
  Synthetic Polypeptide
TNKTKNNHVSDLGLVLCDFE

SEQ ID NO: 47 - protein, 20 aa, Artificial Sequence
  Synthetic Polypeptide
NKTKNNHVSDLGLVLCDFED

SEQ ID NO: 48 - protein, 20 aa, Artificial Sequence
  Synthetic Polypeptide
KTKNNHVSDLGLVLCDFEDS

SEQ ID NO: 49 - protein, 20 aa, Artificial Sequence
  Synthetic Polypeptide
TKNNHVSDLGLVLCDFEDSF

SEQ ID NO: 50 - protein, 20 aa, Artificial Sequence
  Synthetic Polypeptide
KNNHVSDLGLVLCDFEDSFY

SEQ ID NO: 51 - protein, 20 aa, Artificial Sequence
  Synthetic Polypeptide
NNHVSDLGLVLCDFEDSFYL

SEQ ID NO: 52 - protein, 20 aa, Artificial Sequence
  Synthetic Polypeptide
NHVSDLGLVLCDFEDSFYLD

SEQ ID NO: 53 - protein, 20 aa, Artificial Sequence
  Synthetic Polypeptide
HVSDLGLVLCDFEDSFYLDT

SEQ ID NO: 54 - protein, 20 aa, Artificial Sequence
  Synthetic Polypeptide
VSDLGLVLCDFEDSFYLDTQ

SEQ ID NO: 55 - protein, 20 aa, Artificial Sequence
  Synthetic Polypeptide
SDLGLVLCDFEDSFYLDTQS

SEQ ID NO: 56 - protein, 20 aa, Artificial Sequence
  Synthetic Polypeptide
DLGLVLCDFEDSFYLDTQSE

SEQ ID NO: 57 - protein, 20 aa, Artificial Sequence
  Synthetic Polypeptide
LGLVLCDFEDSFYLDTQSEK

SEQ ID NO: 58 - protein, 20 aa, Artificial Sequence
  Synthetic Polypeptide
GLVLCDFEDSFYLDTQSEKI

SEQ ID NO: 59 - protein, 20 aa, Artificial Sequence
  Synthetic Polypeptide
LVLCDFEDSFYLDTQSEKII

SEQ ID NO: 60 - protein, 20 aa, Artificial Sequence
  Synthetic Polypeptide
VLCDFEDSFYLDTQSEKIIQ

SEQ ID NO: 61 - protein, 20 aa, Artificial Sequence
  Synthetic Polypeptide
LCDFEDSFYLDTQSEKIIQQ

SEQ ID NO: 62 - protein, 24 aa, Artificial Sequence
  Synthetic Polypeptide
NLVYSAPTSAGKTLVAELLILKRV

SEQ ID NO: 63 - protein, 24 aa, Artificial Sequence
  Synthetic Polypeptide
KMDLLGMVVVDELHMLGDSHRGYL

SEQ ID NO: 64 - protein, 24 aa, Artificial Sequence
  Synthetic Polypeptide
DCFILMPTGGGKSLCYQLPACVSP

SEQ ID NO: 65 - protein, 24 aa, Artificial Sequence
  Synthetic Polypeptide
ERKLLARFVIDEAHCVSQWGHDFR

SEQ ID NO: 66 - protein, 24 aa, Artificial Sequence
  Synthetic Polypeptide
STLLVLPTGAGKSLCYQLPALLYS

SEQ ID NO: 67 - protein, 24 aa, Artificial Sequence
  Synthetic Polypeptide
QLPPVAFACIDEAHCLSQWSHNFR

SEQ ID NO: 68 - protein, 24 aa, Artificial Sequence
  Synthetic Polypeptide
GAILADEMGLGKTLQCISLIWTLQ

SEQ ID NO: 69 - protein, 24 aa, Artificial Sequence
  Synthetic Polypeptide
KNIKFDLLICDEGHRLKNSAIKTT

SEQ ID NO: 70 - protein, 24 aa, Artificial Sequence
  Synthetic Polypeptide
GCIMADEMGLGKTLQCITLMWTLL

SEQ ID NO: 71 - protein, 24 aa, Artificial Sequence
  Synthetic Polypeptide
QKGSVGLVICDEGHRLKNSENQTY

SEQ ID NO: 72 - protein, 30 aa, Caenorhabditis elegans
SLTRFLDTINLQLVIVENIDAILHDTVYHK

SEQ ID NO: 73 - protein, 38 aa, Homo sapiens
TYTTNKTKNNHVSDLGLVLCDFEDSFYLDTQSEKIIQQ

SEQ ID NO: 74 - DNA, 30 bp, Artificial Sequence
  Synthetic Polynucleotide
gttagcaggtaccgagcaacaattcactgg